SEQUENCE LISTING (40 sequences)

SEQ ID NO: 1 - protein, 22 aa, Unknown
  mammal
YARAAARQARAKALARQLGVAA

SEQ ID NO: 2 - protein, 11 aa, Unknown
  mammal
KALARQLGVAA

SEQ ID NO: 3 - protein, 23 aa, Unknown
  mammal
WLRRIKAWLRRIKALNRQLGVAA

SEQ ID NO: 4 - protein, 21 aa, Unknown
  mammal
FAKLAARLYRKALARQLGVAA

SEQ ID NO: 5 - protein, 23 aa, Unknown
  mammal
KAFAKLAARLYRKALARQLGVAA

SEQ ID NO: 6 - protein, 22 aa, unknown
  mammal
YARAAARQARAKALNRQLGVAA

SEQ ID NO: 7 - protein, 21 aa, unknown
  mammal
YARAAARQARAKALARQLAVA

SEQ ID NO: 8 - protein, 21 aa, unknown
  mammal
YARAAARQARAKALARQLGVA

SEQ ID NO: 9 - protein, 21 aa, unknown
  mammal
YARAAARQARAKALNRQLAVA

SEQ ID NO: 10 - protein, 21 aa, Unknown
  mammal
YARAAARQARAKALNRQLGVA

SEQ ID NO: 11 - protein, 22 aa, unknown
  mammal
YARAAARQARAKALNRQLGVAA

SEQ ID NO: 12 - protein, 22 aa, unknown
  mammal
YARAAARQARAKALNRQLAVAA

SEQ ID NO: 13 - protein, 10 aa, unknown
  mammal
KALARQLAVA

SEQ ID NO: 14 - protein, 10 aa, unknown
  mammal
KALARQLGVA

SEQ ID NO: 15 - protein, 11 aa, unknown
  mammal
KALARQLGVAA

SEQ ID NO: 16 - protein, 11 aa, unknown
  mammal
KALNRQLGVAA

SEQ ID NO: 17 - protein, 11 aa, unknown
  mammal
KAANRQLGVAA

SEQ ID NO: 18 - protein, 11 aa, unknown
  mammal
KALNAQLGVAA

SEQ ID NO: 19 - protein, 11 aa, unknown
  mammal
KALNRALGVAA

SEQ ID NO: 20 - protein, 11 aa, unknown
  mammal
KALNRQAGVAA

SEQ ID NO: 21 - protein, 10 aa, unknown
  mammal
KALNRQLAVA

SEQ ID NO: 22 - protein, 11 aa, unknown
  mammal
KALNRQLAVAA

SEQ ID NO: 23 - protein, 11 aa, unknown
  mammal
KALNRQLGAAA

SEQ ID NO: 24 - protein, 10 aa, unknown
  mammal
KALNRQLGVA

SEQ ID NO: 25 - protein, 13 aa, unknown
  mammal
KKKALNRQLGVAA

SEQ ID NO: 26 - protein, 11 aa, unknown
  mammal
YARAAARQARA

SEQ ID NO: 27 - protein, 14 aa, unknown
  mammal
WLRRIKAWLRRIKA

SEQ ID NO: 28 - protein, 7 aa, unknown
  mammal
WLRRIKA

SEQ ID NO: 29 - protein, 11 aa, unknown
  mammal
YGRKKRRQRRR

SEQ ID NO: 30 - protein, 12 aa, unknown
  mammal
WLRRIKAWLRRI

SEQ ID NO: 31 - protein, 10 aa, unknown
  mammal
FAKLAARLYR

SEQ ID NO: 32 - protein, 12 aa, unknown
  mammal
KAFAKLAARLYR

SEQ ID NO: 33 - protein, 9 aa, unknown
  mammal
RKKRRQRRR

SEQ ID NO: 34 - protein, 10 aa, unknown
  mammal
KKLNRTLSVA

SEQ ID NO: 35 - protein, 10 aa, unknown
  mammal
KKLRRTLSVA

SEQ ID NO: 36 - protein, 216 aa, unknown
  mammal
YARAAARQARAMTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 37 - protein, 16 aa, unknown
  mammal
KKXXXXXXXXXXKRKK

SEQ ID NO: 38 - protein, 7 aa, unknown
  mammal
LLKRRKK

SEQ ID NO: 39 - protein, 6 aa, unknown
  mammal
XXRXXX

SEQ ID NO: 40 - protein, 10 aa, unknown
  mammal
MXXXLXXMXV

(19) United States
(12) Patent Application Publication
(10) Pub. No.: US 2011/0288036 A1
(43) Pub. Date: Nov. 24, 2011

(54) Methods for Treating or Preventing Vascular Graft Failure

(76) Inventors: Cynthia Lander, Mendham, NJ (US); Colleen Brophy, Nashville, TN (US); Brandon Seal, Pleasant Grove, UT (US)

(21) Appl. No.: 13/114,872

(22) Filed: May 24, 2011

Related U.S. Application Data
(60) Provisional application No. 61/347,495, filed on May 24, 2010.

(51) Int. Cl. A61K 38/16; A61K 38/08; A61P 9/00; A61K 38/10

(52) U.S. Cl. 514/21.4; 514/21.6; 514/21.5; 514/21.7

(57) ABSTRACT

The described invention provides pharmaceutical compositions and methods for treating or preventing vascular graft failure in a subject in need of such treatment, the method comprising administering a therapeutically effective amount of a composition comprising a polypeptide of amino acid sequence YARAAARQARAKALARQLGVAA (SEQ ID NO: 1) or a functional equivalent thereof, and a pharmaceutically acceptable carrier. The methods also are clinically useful for treating a pre-atherosclerotic intimal hyperplasia condition.

### CROSS REFERENCE TO RELATED APPLICATIONS

This application claims the benefit of priority of U.S. Application No. 61/347,495, filed May 24, 2010, and U.S. application Ser. No. 11/972,459, filed Jan. 10, 2008, which claims priority to U.S. Provisional Application No. 60/880,137, filed Jan. 10, 2007. Each of these applications is incorporated by reference herein in its entirety.

### FIELD OF THE INVENTION

The invention is in the fields of cell and molecular biology, polypeptides, and therapeutic methods of use.

### BACKGROUND OF THE INVENTION

1. Kinases

Kinases are a ubiquitous group of enzymes that catalyze the phosphoryl transfer reaction from a phosphate donor (usually adenosine-5′-triphosphate (ATP)) to a receptor substrate. Although all kinases catalyze essentially the same phosphoryl transfer reaction, they display remarkable diversity in their substrate specificity, structure, and the pathways in which they participate. A recent classification of all available kinase sequences (approximately 60,000 sequences) indicates kinases can be grouped into 25 families of homologous (meaning derived from a common ancestor) proteins. These kinase families are assembled into 12 fold groups based on similarity of structural fold. Further, 22 of the 25 families (approximately 98.8% of all sequences) belong to 10 fold groups for which the structural fold is known. Of the other 3 families, polyphosphate kinase forms a distinct fold group, and the 2 remaining families are both integral membrane kinases and comprise the final fold group. These fold groups not only include some of the most widely spread protein folds, such as Rossmann-like fold (three or more parallel β strands linked by two α helices in the topological order β-α-β-α-β), ferredoxin-like fold (a common α+β protein fold with a signature βαββαβ secondary structure along its backbone), TIM-barrel fold (meaning a conserved protein fold consisting of eight α-helices and eight parallel β-strands that alternate along the peptide backbone), and anti-parallel β-barrel fold (a beta barrel is a large beta-sheet that twists and coils to form a closed structure in which the first strand is hydrogen bonded to the last), but also all major classes (all α, all β, α+β, α/β) of protein structures. Within a fold group, the core of the nucleotide-binding domain of each family has the same architecture, and the topology of the protein core is either identical or related by circular permutation. Homology between the families within a fold group is not implied.

Group I (23,124 sequences) kinases incorporate protein S/T-Y kinase, atypical protein kinase, lipid kinase, and ATP grasp enzymes and further comprise the protein S/T-Y kinase, and atypical protein kinase family (22,074 sequences). These kinases include: choline kinase (EC 2.7.1.32); protein kinase (EC 2.7.137); phosphorylase kinase (EC 2.7.1.38); homoserine kinase (EC 2.7.1.39); 1-phosphatidylinositol 4-kinase (EC 2.7.1.67); streptomycin 6-kinase (EC 2.7.1.72); ethanolamine kinase (EC 2.7.1.82); streptomycin 3′-kinase (EC 2.7.1.87); kanamycin kinase (EC 2.7.1.95); 5-methylthioribose kinase (EC 2.7.1.100); viomycin kinase (EC 2.7.1.103); hydroxymethylglutaryl-CoA reductase (NADPH2) kinase (EC 2.7.1.109); protein-tyrosine kinase (EC 2.7.1.112); isocitrate dehydrogenase (NADP⁺) kinase (EC 2.7.1.116); myosin light-chain kinase (EC 2.7.1.117); hygromycin-B kinase (EC 2.7.1.119); calcium/calmodulin-dependent protein kinase (EC 2.7.1.123); rhodopsin kinase (EC 2.7.1.125); beta-adrenergic-receptor kinase (EC 2.7.1.126); myosin heavy-chain kinase (EC 2.7.1.129); Tau protein kinase (EC 2.7.1.135); macrolide 2′-kinase (EC 2.7.1.136); I-phosphatidylinositol 3-kinase (EC 2.7.1.137); RNA-polymerase-subunit kinase (EC 2.7.1.141); phosphatidylinositol-4,5-bisphosphate 3-kinase (EC 2.7.1.153); and phosphatidylinositol-4-phosphate 3-kinase (EC 2.7.1.154). Group I further comprises the lipid kinase family (321 sequences). These kinases include: I-phosphatidylinositol-4-phosphate 5-kinase (EC 2.7.1.68); I D-myo-inositol-triphosphate 3-kinase (EC 2.7.1.127); inositol-tetrakisphosphate 5-kinase (EC 2.7.1.140); 1-phosphatidylinositol-5-phosphate 4-kinase (EC 2.7.1.149); 1-phosphatidylinositol-3-phosphate 5-kinase (EC 2.7.1.150); inositol-polyphosphate multikinase (EC 2.7.1.151); and inositol-hexakiphosphate kinase (EC 2.7.4.21). Group I further comprises the ATP-grasp kinases (729 sequences) which include inositol-tetrakisphosphate I-kinase (EC 2.7.1.134); pyruvate, phosphate dikinase (EC 2.7.9.1); and pyruvate, water dikinase (EC 2.7.9.2).

Group II (17,071 sequences) kinases incorporate the Rossman-like kinases. Group II comprises the P-loop kinase family (7,732 sequences). These include gluconokinase (EC 2.7.1.12); phosphoribulokinase (EC 2.7.1.19); thymidine kinase (EC 2.7.1.21); ribosylnicotinamide kinase (EC 2.7.1.22); dephospho-CoA kinase (EC 2.7.1.24); adenylylsulfate kinase (EC 2.7.1.25); pantothenate kinase (EC 2.7.1.33); protein kinase (bacterial) (EC 2.7.1.37); uridine kinase (EC 2.7.1.48); shikimate kinase (EC 2.7.1.71); deoxycytidine kinase (EC 2.7.1.74); deoxyadenosine kinase (EC 2.7.1.76); polynucleotide 5′-hydroxyl-kinase (EC 2.7.1.78); 6-phosphofructo-2-kinase (EC 2.7.1.105); deoxyguanosine kinase (EC 2.7.1.113); tetraacyldisaccharide 4′-kinase (EC 2.7.1.130); deoxynucleoside kinase (EC 2.7.1.145); adenosylcobinamide kinase (EC 2.7.1.156); polyphosphate kinase (EC 2.7.4.1); phosphomevalonate kinase (EC 2.7.4.2); adenylate kinase (EC 2.7.4.3); nucleoside-phosphate kinase (EC 2.7.4.4); guanylate kinase (EC 2.7.4.8); thymidylate kinase (EC 2.7.4.9); nucleoside-triphosphate-adenylate kinase (EC 2.7.4.10); (deoxy)nucleoside-phosphate kinase (EC 2.7.4.13); cytidylate kinase (EC 2.7.4.14); and uridylate kinase (EC 2.7.4.22). Group II further comprises the phosphoenolpyruvate carboxykinase family (815 sequences). These enzymes include protein kinase (HPr kinase/phosphatase) (EC 2.7.1.37); phosphoenolpyruvate carboxykinase (GTP) (EC 4.1.1.32); and phosphoenolpyruvate carboxykinase (ATP) (EC 4.1.1.49). Group II further comprises the phosphoglycerate kinase (1,351 sequences) family. These enzymes include phosphoglycerate kinase (EC 2.7.2.3) and phosphoglycerate kinase (GTP) (EC 2.7.2.10). Group II further comprises the aspartokinase family (2,171 sequences). These enzymes include carbamate kinase (EC 2.7.2.2); aspartate kinase (EC 2.7.2.4); acetylglutamate kinase (EC 2.7.2.8 1); glutamate 5-kinase (EC 2.7.2.1) and uridylate kinase (EC 2.7.4.). Group II further comprises the phosphofructokinase-like kinase family (1,998 sequences). These enzymes include 6-phosphofrutokinase (EC 2.7.1.1 1); NAD (+) kinase (EC 2.7.1.23); 1-phosphofructokinase (EC 2.7.1.56); diphosphate-fructose-6-phosphate I-phosphotransferase (EC 2.7.1.90); sphinganine kinase (EC 2.7.1.91); diacylglycerol kinase (EC 2.7.1.107); and ceramide kinase (EC 2.7.1.138). Group II further comprises the ribokinase-like family (2,722 sequences). These enzymes include: glucokinase (EC 2.7.1.2); ketohexokinase (EC 2.7.1.3); fructokinase (EC 2.7.1.4); 6-phosphofructokinase (EC 2.7.1.11); ribokinase (EC 2.7.1.15); adenosine kinase (EC 2.7.1.20); pyridoxal kinase (EC 2.7.1.35); 2-dehydro-3-deoxygluconokinase (EC 2.7.1.45); hydroxymethylpyrimidine kinase (EC 2.7.1.49); hydroxyethylthiazole kinase (EC 2.7.1.50); 1-phosphofructokinase (EC 2.7.1.56); inosine kinase (EC 2.7.1.73); 5-dehydro-2-deoxygluconokinase (EC 2.7.1.92); tagatose-6-phosphate kinase (EC 2.7.1.144); ADP-dependent phosphofructokinase (EC 2.7.1.146); ADP-dependent glucokinase (EC 2.7.1.147); and phosphomethylpyrimidine kinase (EC 2.7.4.7). Group II further comprises the thiamin pyrophosphokinase family (175 sequences) which includes thiamin pyrophosphokinase (EC 2.7.6.2). Group II further comprises the glycerate kinase family (107 sequences) which includes glycerate kinase (EC 2.7.1.31).

Group III kinases (10,973 sequences) comprise the ferredoxin-like fold kinases. Group III further comprises the nucleoside-diphosphate kinase family (923 sequences). These enzymes include nucleoside-diphosphate kinase (EC 2.7.4.6). Group III further comprises the HPPK kinase family (609 sequences). These enzymes include 2-amino-4-hydroxy-6-hydroxymethyldihydropteridine pyrophosphokinase (EC 2.7.6.3). Group III further comprises the guanido kinase family (324 sequences). These enzymes include guanidoacetate kinase (EC 2.7.3.1); creatine kinase (EC 2.7.3.2); arginine kinase (EC 2.7.3.3); and lombricine kinase (EC 2.7.3.5). Group III further comprises the histidine kinase family (9,117 sequences). These enzymes include protein kinase (histidine kinase) (EC 2.7.1.37); [pyruvate dehydrogenase (lipoamide)]kinase (EC 2.7.1.99); and [3-methyl-2-oxybutanoate dehydrogenase(lipoamide)]kinase (EC 2.7.1.115).

Group IV kinases (2,768 sequences) incorporate ribonuclease H-like kinases. These enzymes include hexokinase (EC 2.7.1.1); glucokinase (EC 2.7.1.2); fructokinase (EC 2.7.1.4); rhamnulokinase (EC 2.7.1.5); mannokinase (EC 2.7.1.7); gluconokinase (EC 2.7.1.12); L-ribulokinase (EC 2.7.1.16); xylulokinase (EC 2.7.1.17); erythritol kinase (EC 2.7.1.27); glycerol kinase (EC 2.7.1.30); pantothenate kinase (EC 2.7.1.33); D-ribulokinase (EC 2.7.1.47); L-fucolokinase (EC 2.7.1.51); L-xylulokinase (EC 2.7.1.53); allose kinase (EC 2.7.1.55); 2-dehydro-3-deoxygalactonokinase (EC 2.7.1.58); N-acetylglucosamine kinase (EC 2.7.1.59); N-acylmannosamine kinase (EC 2.7.1.60); polyphosphate-glucose phosphotransferase (EC 2.7.1.63); beta-glucoside kinase (EC 2.7.1.85); acetate kinase (EC 2.7.2.1); butyrate kinase (EC 2.7.2.7); branched-chain-fatty-acid kinase (EC 2.7.2.14); and propionate kinase (EC 2.7.2.15).

Group V kinases (1,119 sequences) incorporate TIM β-barrel kinases. These enzymes include pyruvate kinase (EC 2.7.1.40).

Group VI kinases (885 sequences) incorporate GHMP kinases. These enzymes include galactokinase (EC 2.7.1.6); mevalonate kinase (EC 2.7.1.36); homoserine kinase (EC 2.7.1.39); L-arabinokinase (EC 2.7.1.46); fucokinase (EC 2.7.1.52); shikimate kinase (EC 2.7.1.71); 4-(cytidine 5′-diphospho)-2-C-methyl-D-erythriol kinase (EC 2.7.1.148); and phosphomevalonate kinase (EC 2.7.4.2).

Group VII kinases (1,843 sequences) incorporate AIR synthetase-like kinases. These enzymes include thiamine-phosphate kinase (EC 2.7.4.16) and selenide, water dikinase (EC 2.7.9.3).

Group VIII kinases (565 sequences) incorporate riboflavin kinases (565 sequences). These enzymes include riboflavin kinase (EC 2.7.1.26).

Group IX kinases (197 sequences) incorporate dihydroxyacetone kinases. These enzymes include glycerone kinase (EC 2.7.1.29).

Group X kinases (148 sequences) incorporate putative glycerate kinases. These enzymes include glycerate kinase (EC 2.7.1.31).

Group XI kinases (446 sequences) incorporate polyphosphate kinases. These enzymes include polyphosphate kinases (EC 2.7.4.1).

Group XII kinases (263 sequences) incorporate integral membrane kinases. Group XII comprises the dolichol kinase family. These enzymes include dolichol kinases (EC 2.7.1.108). Group XII further comprises the undecaprenol kinase family. These enzymes include undecaprenol kinases (EC 2.7.1.66).

Kinases play indispensable roles in numerous cellular metabolic and signaling pathways, and they are among the best-studied enzymes at the structural, biochemical, and cellular levels. Despite the fact that all kinases use the same phosphate donor (in most cases, ATP) and catalyze apparently the same phosphoryl transfer reaction, they display remarkable diversity in their structural folds and substrate recognition mechanisms. This probably is due largely to the extraordinary diverse nature of the structures and properties of their substrates.

1.1. Mitogen-Activated Protein Kinase-Activated Protein Kinases (MK2 and MK3)

Different groups of MAPK-activated protein kinases (MAP-KAPKs) have been defined downstream of mitogen-activated protein kinases (MAPKs). These enzymes transduce signals to target proteins that are not direct substrates of the MAPKs and, therefore, serve to relay phosphorylation-dependent signaling with MAPK cascades to diverse cellular functions. One of these groups is formed by the three MAPKAPKs: MK2, MK3 (also known as 3pK), and MK5 (also designated PRAK). Mitogen-activated protein kinase-activated protein kinase 2 (also referred to as “MAPKAPK2”, “MAPKAP-K2”, “MK2”) is a kinase of the serine/threonine (Ser/Thr) protein kinase family. MK2 is highly homologous to MK3 (approximately 75% amino acid identity). The kinase domains of MK2 and MK3 are most similar (approximately 35% to 40% identity) to calcium/calmodulin-dependent protein kinase (CaMK), phosphorylase b kinase, and the C-terminal kinase domain (CTKD) of the ribosomal S6 kinase (RSK) isoforms. The mk2 gene encodes two alternatively spliced transcripts of 370 amino acids (MK2A) and 400 amino acids (MK2B). The mk3 gene encodes one transcript of 382 amino acids. The MK2 and MK3 proteins are highly homologous, yet MK2A possesses a shorter C-terminal region. The C-terminus of MK2B contains a functional bipartite nuclear localization sequence (NLS) (Lys-Lys-Xaa₁₀-Lys-Arg-Arg-Lys-Lys (SEQ ID NO: 37)) that is not present in the shorter MK2A isoform, indicating that alternative splicing determines the cellular localization of the MK2 isoforms. MK3 possesses a similar nuclear localization sequence. The nuclear localization sequence found in both MK2B and MK3 encompasses a D domain (Leu-Leu-Lys-Arg-Arg-Lys-Lys (SEQ ID NO: 38)) that studies have shown to mediate the specific interaction of MK2B and MK3 with p38α and p38β. MK2B and MK3 also possess a functional nuclear export signal (NES, SEQ ID NO: 40, Met-Xaa-Xaa-Xaa-Leu-Xaa-Xaa-Met-Xaa-Val) located N-terminal to the NLS and D domain. The NES in MK2B is sufficient to trigger nuclear export following stimulation, a process which may be inhibited by leptomycin B. The sequence N-terminal to the catalytic domain in MK2 and MK3 is proline rich and contains one (MK3) or two (MK2) putative Src homology 3 (SH3) domain-binding sites, which studies have shown, for MK2, to mediate binding to the SH3 domain of c-Abl in vitro. Recent studies suggest that this domain is involved in MK2-mediated cell migration.

MK2B and MK3 are located predominantly in the nucleus of quiescent cells while MK2A is present in the cytoplasm. Both MK2B and MK3 are rapidly exported to the cytoplasm via a chromosome region maintenance protein (CRM1)-dependent mechanism upon stress stimulation. Nuclear export of MK2B appears to be mediated by kinase activation, as phosphomimetic mutation of Thr334 within the activation loop of the kinase enhances the cytoplasmic localization of MK2B. Without being limited by theory, it is thought that MK2B and MK3 may contain a constitutively active NLS and a phosphorylation-regulated NES.

MK2 and MK3 appear to be expressed ubiquitously, with predominant expression in the heart, in skeletal muscle, and in kidney tissues.

1.1.1. Activation

Various activators of p38α and p38β potently stimulate MK2 and MK3 activity. p38 mediates the in vitro and in vivo phosphorylation of MK2 on four proline-directed sites: Thr25, Thr222, Ser272, and Thr334. Of these sites, only Thr25 is not conserved in MK3. Without being limited by theory, while the function of phosphorylated Thr25 in unknown, its location between the two SH3 domain-binding sites suggests that it may regulate protein-protein interactions. Thr222 in MK2 (Thr201 in MK3) is located in the activation loop of the kinase domain and has been shown to be essential for MK2 and MK3 kinase activity. Thr334 in MK2 (Thr313 in MK3) is located C-terminal to the catalytic domain and is essential for kinase activity. The crystal structure of MK2 has been resolved and, without being limited by theory, suggests that Thr334 phosphorylation may serve as a switch for MK2 nuclear import and export. Phosphorylation of Thr334 also may weaken or interrupt binding of the C terminus of MK2 to the catalytic domain, exposing the NES and promoting nuclear export.

Studies have shown that, while p38 is capable of activating MK2 and MK3 in the nucleus, experimental evidence suggests that activation and nuclear export of MK2 and MK3 are coupled by a phosphorylation-dependent conformational switch that also dictates p38 stabilization and localization, and the cellular location of p38 itself is controlled by MK2 and possibly MK3. Additional studies have shown that nuclear p38 is exported to the cytoplasm in a complex with MK2 following phosphorylation and activation of MK2. The interaction between p38 and MK2 may be important for p38 stabilization since studies indicate that p38 levels are low in MK2-deficient cells and expression of a catalytically inactive MK2 protein restores p38 levels.

1.1.2. Substrates and Functions

MK2 shares many substrates with MK3. Both enzymes have comparable substrate preferences and phosphorylate peptide substrates with similar kinetic constants. The minimum sequence required for efficient phosphorylation by MK2 was found to be Hyd-Xaa-Arg-Xaa-Xaa-pSer/Thr (SEQ ID NO: 39), where Hyd is a bulky hydrophobic residue selected from the group consisting of Leu, Ile, Val, Met, Phe, and Trp.

Experimental evidence supports a role for p38 in the regulation of cytokine biosynthesis and cell migration. The targeted deletion of the mk2 gene in mice suggested that although p38 mediates the activation of many similar kinases, MK2 seems to be the key kinase responsible for these p38-dependent biological processes. Loss of MK2 leads (i) to a defect in lipopolysaccharide (LPS)-induced synthesis of cytokines such as tumor necrosis factor alpha (TNF-α), interleukin-6 (IL-6), and gamma interferon (IFN-γ) and (ii) to changes in the migration of mouse embryonic fibroblasts, smooth muscle cells, and neutrophils. Consistent with a role for MK2 in inflammatory responses, MK2-deficient mice show increased susceptibility to Listeria monocytogenes infection and reduced inflammation-mediated neuronal death following focal ischemia. Since the levels of p38 protein also are reduced significantly in MK2-deficient cells, it was necessary to distinguish whether these phenotypes were due solely to the loss of MK2. To achieve this, MK2 mutants were expressed in MK2-deficient cells, and the results indicated that the catalytic activity of MK2 was not necessary to restore p38 levels but was required to regulate cytokine biosynthesis.

1.1.3. Regulation of mRNA Translation.

Previous studies using MK2 knockout mice or MK2-deficient cells have shown that MK2 increases the production of inflammatory cytokines, including TNF-α, IL-1, and IL-6, by increasing the rate of translation of its mRNA. No significant reductions in the transcription, processing, and shedding of TNF-α could be detected in MK2-deficient mice. The p38 pathway is known to play an important role in regulating mRNA stability, and MK2 represents a likely target by which p38 mediates this function. Studies utilizing MK2-deficient mice indicated that the catalytic activity of MK2 is necessary for its effects on cytokine production and migration, suggesting that, without being limited by theory, MK2 phosphorylates targets involved in mRNA stability. Consistent with this, MK2 has been shown to bind and/or phosphorylate the heterogeneous nuclear ribonucleoprotein (hnRNP) A0, tristetraprolin, the poly(A)-binding protein PABP1, and HuR (a ubiquitously expressed member of the elav (embryonic-lethal abnormal visual in Drosophila melanogaster) family of RNA-binding protein). These substrates are known to bind or co-purify with mRNAs that contain AU-rich elements in the 3′ untranslated region, suggesting that MK2 may regulate the stability of AU-rich mRNAs such as TNF-α. It currently is unknown whether MK3 plays similar functions, but LPS treatment of MK2-deficient fibroblasts completely abolished hnRNP A0 phosphorylation, suggesting that MK3 is not able to compensate for the loss of MK2.

MK3 participates with MK2 in phosphorylation of the eukaryotic elongation factor 2 (eEF2) kinase. eEF2 kinase phosphorylates and inactivates eEF2. eEF2 activity is critical for the elongation of mRNA during translation, and phosphorylation of eEF2 on Thr56 results in the termination of mRNA translation. MK2 and MK3 phosphorylation of eEF2 kinase on Ser377 suggests that these enzymes may modulate eEF2 kinase activity and thereby regulate mRNA translation elongation.

1.1.4. Transcriptional Regulation by MK2 and MK3.

Nuclear MK2, similar to many MKs, contributes to the phosphorylation of cAMP response element binding (CREB), serum response factor (SRF), and transcription factor ER81. Comparison of wild-type and MK2-deficient cells revealed that MK2 is the major SRF kinase induced by stress, suggesting a role for MK2 in the stress-mediated immediate-early response. Both MK2 and MK3 interact with basic helix-loop-helix transcription factor E47 in vivo and phosphorylate E47 in vitro. MK2-mediated phosphorylation of E47 was found to repress the transcriptional activity of E47 and thereby inhibit E47-dependent gene expression, suggesting that MK2 and MK3 may regulate tissue-specific gene expression and cell differentiation.

1.1.5. Other Targets of MK2 and MK3.

Several other MK2 and MK3 substrates also have been identified, reflective of the diverse functions of MK2 and MK3 in several biological processes. The scaffolding protein 14-3-3ζ is a physiological MK2 substrate. Studies indicate 14-3-3ζ interacts with a number of components of cell signaling pathways, including protein kinases, phosphatases, and transcription factors. Additional studies have shown that MK2-mediated phosphorylation of 14-3-3ζ on Ser58 compromises its binding activity, suggesting that MK2 may affect the regulation of several signaling molecules normally regulated by 14-3-3ζ.

Additional studies have shown that MK2 also interacts with and phosphorylates the p16 subunit of the seven-member Arp2 and Arp3 complex (p16-Arc) on Ser77. p16-Arc has roles in regulating the actin cytoskeleton, suggesting that MK2 may be involved in this process. Further studies have shown that the small heat shock protein HSPB1, lymphocyte-specific protein LSP-1, and vimentin are phosphorylated by MK2. HSPB1 is of particular interest because it forms large oligomers which may act as molecular chaperones and protect cells from heat shock and oxidative stress. Upon phosphorylation, HSPB1 loses its ability to form large oligomers and is unable to block actin polymerization, suggesting that MK2-mediated phosphorylation of HSPB1 serves a homeostatic function aimed at regulating actin dynamics that otherwise would be destabilized during stress. MK3 also was shown to phosphorylate HSPB1 in vitro and in vivo, but its role during stressful conditions has not yet been elucidated.

MK2 and MK3 also may phosphorylate 5-lipoxygenase. 5-lipoxygenase catalyzes the initial steps in the formation of the inflammatory mediators leukotrienes. Tyrosine hydroxylase, glycogen synthase, and Akt also were shown to be phosphorylated by MK2. Finally, MK2 phosphorylates the tumor suppressor protein tuberin on Ser1210, creating a docking site for 14-3-3ζ. Tuberin and hamartin normally form a functional complex that negatively regulates cell growth by antagonizing mTOR-dependent signaling, suggesting that p38-mediated activation of MK2 may regulate cell growth by increasing 14-3-3ζ binding to tuberin.

1.2. Kinase Inhibition

The eukaryotic protein kinases constitute one of the largest superfamilies of homologous proteins that are related by virtue of their catalytic domains. Most related protein kinases are specific for either serine/threonine or tyrosine phosphorylation. Protein kinases play an integral role in the cellular response to extracellular stimuli. Thus, stimulation of protein kinases is considered to be one of the most common activation mechanisms in signal transduction systems. Many substrates are known to undergo phosphorylation by multiple protein kinases. A considerable amount of information on primary sequence of the catalytic domains of various protein kinases has been published. These sequences share a large number of residues involved in ATP binding, catalysis, and maintenance of structural integrity. Most protein kinases possess a well conserved 30-32 kDa catalytic domain.

Studies have attempted to identify and utilize regulatory elements of protein kinases. These regulatory elements include inhibitors, antibodies, and blocking peptides.

1.2.1. Inhibitors

Enzyme inhibitors are molecules that bind to enzymes thereby decreasing enzyme activity. The binding of an inhibitor may stop a substrate from entering the active site of the enzyme and/or hinder the enzyme from catalyzing its reaction Inhibitor binding is either reversible or irreversible. Irreversible inhibitors usually react with the enzyme and change it chemically (e.g., by modifying key amino acid residues needed for enzymatic activity) so that it no longer is capable of catalyzing its reaction. In contrast, reversible inhibitors bind non-covalently and different types of inhibition are produced depending on whether these inhibitors bind the enzyme, the enzyme-substrate complex, or both.

Enzyme inhibitors often are evaluated by their specificity and potency. The term “specificity” as used in this context refers to the selective attachment of an inhibitor or its lack of binding to other proteins. The term “potency” as used herein refers to an inhibitor's dissociation constant, which indicates the concentration of inhibitor needed to inhibit an enzyme.

Inhibitors of protein kinases have been studied for use as a tool in protein kinase activity regulation Inhibitors have been studied for use with, for example, cyclin-dependent (Cdk) kinase, MAP kinase, serine/threonine kinase, Src Family protein tyrosine kinase, tyrosine kinase, calmodulin (CaM) kinase, casein kinase, checkpoint kinase (Chk1), glycogen synthase kinase 3 (GSK-3), c-Jun N-terminal kinase (JNK), mitogen-activated protein kinase 1 (MEK), myosin light chain kinase (MLCK), protein kinase A, Akt (protein kinase B), protein kinase C, protein kinase G, protein tyrosine kinase, Raf kinase, and Rho kinase.

1.2.2. Blocking Peptides

A peptide is a chemical compound that is composed of a chain of two or more amino acids whereby the carboxyl group of one amino acid in the chain is linked to the amino group of the other via a peptide bond. Peptides have been used inter alia in the study of protein structure and function. Synthetic peptides may be used inter alia as probes to see where protein-peptide interactions occur Inhibitory peptides may be used inter alia in clinical research to examine the effects of peptides on the inhibition of protein kinases, cancer proteins and other disorders.

The use of several blocking peptides has been studied. For example, extracellular signal-regulated kinase (ERK), a MAPK protein kinase, is essential for cellular proliferation and differentiation. The activation of MAPKs requires a cascade mechanism whereby MAPK is phosphorylated by an upstream MAPKK (MEK) which then, in turn, is phosphorylated by a third kinase MAPKKK (MEKK). The ERK inhibitory peptide functions as a MEK decoy by binding to ERK.

Other blocking peptides include autocamtide-2 related inhibitory peptide (AIP). This synthetic peptide is a highly specific and potent inhibitor of Ca²⁺/calmodulin-dependent protein kinase II (CaMKII). AIP is a non-phosphorylatable analog of autocamtide-2, a highly selective peptide substrate for CaMKII. AIP inhibits CaMKII with an IC₅₀ of 100 nM (IC₅₀ is the concentration of an inhibitor required to obtain 50% inhibition). The AIP inhibition is non-competitive with respect to syntide-2 (CaMKII peptide substrate) and ATP but competitive with respect to autocamtide-2. The inhibition is unaffected by the presence or absence of Ca²⁺/calmodulin. CaMKII activity is inhibited completely by AIP (1 μM) while PKA, PKC and CaMKIV are not affected.

Other blocking peptides include cell division protein kinase 5 (Cdk5) inhibitory peptide (CIP). Cdk5 phosphorylates the microtubule protein tau at Alzheimer's Disease-specific phospho-epitopes when it associates with p25. p25 is a truncated activator, which is produced from the physiological Cdk5 activator p35 upon exposure to amyloid β peptides. Upon neuronal infections with CIP, CIPs selectively inhibit p25/Cdk5 activity and suppress the aberrant tau phosphorylation in cortical neurons. The reasons for the specificity demonstrated by CIP are not fully understood.

Additional blocking peptides have been studied for extracellular-regulated kinase 2 (ERK2), ERK3, p38/HOG1, protein kinase C, casein kinase II, Ca²⁺/calmodulin kinase IV, casein kinase II, Cdk4, Cdk5, DNA-dependent protein kinase (DNA-PK), serine/threonine-protein kinase PAK3, phosphoinositide (PI)-3 kinase, PI-5 kinase, PSTAIRE (the cdk highly conserved sequence), ribosomal S6 kinase, GSK-4, germinal center kinase (GCK), SAPK (stress-activated protein kinase), SEK1 (stress signaling kinase), and focal adhesion kinase (FAK).

1.2.3. Protein Transduction Domains

Protein transduction domains (PTDs) are a class of peptides capable of penetrating the plasma membrane of mammalian cells and of transporting compounds of many types and molecular weights across the membrane. These compounds include effector molecules, such as proteins, DNA, conjugated peptides, oligonucleotides, and small particles such as liposomes. When PTDs are chemically linked or fused to other proteins, the resulting fusion proteins still are able to enter cells. Although the exact mechanism of transduction is unknown, internalization of these proteins is not believed to be receptor-mediated or transporter-mediated. PTDs are generally 10-16 amino acids in length and may be grouped according to their composition, such as, for example, peptides rich in arginine and/or lysine.

The use of PTDs capable of transporting effector molecules into cells has become increasingly attractive in the design of drugs as they promote the cellular uptake of cargo molecules. These cell-penetrating peptides, generally categorized as amphipathic (meaning having both a polar and a nonpolar end) or cationic (meaning of or relating to containing net positively charged atoms) depending on their sequence, provide a non-invasive delivery technology for macromolecules. PTDs often are referred to as “Trojan peptides”, “membrane translocating sequences”, or “cell permeable proteins” (CPPs). PTDs also may be used to assist novel HSPB1 kinase inhibitors to penetrate cell membranes (see U.S. application Ser. No. 11/972,459, entitled “Polypeptic Inhibitors of HSPB1 Kinase and Uses Thereof,” filed Jan. 10, 2008, and Ser. No. 12/188,109, entitled “Kinase Inhibitors and Uses Thereof,” filed Aug. 7, 2008, incorporated by reference in their entirety herein.

1.2.3.1. Viral PTD Containing Proteins

The first proteins to be described as having transduction properties were of viral origin. These proteins still are the most commonly accepted models for PTD action. The HIV-1 Transactivator of Transcription (TAT) and HSV-1 VP 22 protein are the best characterized viral PTD containing proteins.

TAT (HIV-1 trans-activator gene product) is an 86-amino acid polypeptide, which acts as a powerful transcription factor of the integrated HIV-1 genome. TAT acts on the viral genome stimulating viral replication in latently infected cells. The translocation properties of the TAT protein enable it to activate quiescent infected cells, and it may be involved in priming of uninfected cells for subsequent infection by regulating many cellular genes, including cytokines. The minimal PTD of TAT is the 9 amino acid protein sequence RKKRRQRRR (TAT49-57) (SEQ ID NO: 33). Studies utilizing a longer fragment of TAT demonstrated successful transduction of fusion proteins up to 120 kDa. The addition of multiple TAT-PTDs as well as synthetic TAT derivatives have been demonstrated to mediate membrane translocation. TAT-PTD containing fusion proteins have been used as therapeutic moieties in experiments involving cancer, transporting a death-protein into cells, and disease models of neurodegenerative disorders.

VP22 is the HSV-1 tegument protein, a structural part of the HSV virion. VP22 is capable of receptor independent translocation and accumulates in the nucleus. This property of VP22 classifies the protein as a PTD containing peptide. Fusion proteins comprising full length VP22 have been translocated efficiently across the plasma membrane.

1.2.3.2. Homeoproteins with Intercellular Translocation Properties

Homeoproteins are highly conserved, transactivating transcription factors involved in morphological processes. They bind to DNA through a specific sequence of 60 amino acids. The DNA-binding homeodomain is the most highly conserved sequence of the homeoprotein. Several homeoproteins have been described to exhibit PTD-like activity; they are capable of efficient translocation across cell membranes in an energy-independent and endocytosis-independent manner without cell type specificity.

The Antennapedia protein (Antp) is a trans-activating factor capable of translocation across cell membranes; the minimal sequence capable of translocation is a 16 amino acid peptide corresponding to the third helix of the protein's homeodomain (HD). The internalization of this helix occurs at 4° C., suggesting that this process is not endocytosis dependent. Peptides of up to 100 amino acids produced as fusion proteins with AntpHD penetrate cell membranes. Other homeodomains capable of translocation include Fushi tarazu (Ftz) and Engrailed (En) homeodomain. Many homeodomains share a highly conserved third helix.

2. Grafts, Vascular Grafts, and Vascular Grafts Failure

A graft is a tissue or organ used for transplantation to a patient. The term “graft” includes, but is not limited to, a self tissue transferred from one body site to another in the same individual (“autologous graft”), a tissue transferred between genetically identical individuals (“syngeneic graft”) or between genetically different members of the same species (“allogeneic graft” or “allograft”), and a tissue transferred between different species (“xenograft”). In addition, a “prosthetic”, “synthetic”, or an “engineered tissue graft”, which is manufactured with artificial materials. Several different materials (natural, synthetic, biodegradable, and permanent) have been used for grafts, some of which have been tissue-engineered to incorporate additional features such as synthetic manufacture, biocompatibility, non-immunogenicity, and nanoscale fibers.

For example, polytetrafluoroethylene (PTFE) and polyester (Dacron®), or with biodegradable materials, e.g., poly(lactic-co-glycolic acid) (PLGA), polylactic acid (PLA), polyglycolic acid (PGA), and polycaprolactone (PCL) may be used for transplantation. PLA is a polyester which degrades within the human body to form a lactic acid byproduct which then is easily eliminated. Polyglycolic acid (PGA) exhibits a faster rate of degradation to lactic acid than PLA, and polycaprolactone (PCL); exhibits a slower rate of degradation to lactic acid than PLA.

Grafts also may be constructed from natural materials. For example, several components of the extracellular matrix have been studied to evaluate their ability to support cell growth. Protein-based materials, such as collagen or fibrin, and polysaccharidic materials, such as chitosan or glycosaminoglycans (GAGs), have proved suitable in terms of cell compatibility; however, there are some concerns with the potential immunogenicity of such materials.

Transplanted grafts often are rejected by the host via an orchestrated immune response against the histocompatibility antigens expressed by the grafted tissue. Effectors primarily responsible for such rejections include type 1 helper CD4+ cells, cytotoxic CD8+ cells and antibodies. Alternative mechanisms of rejection include the involvement of type 2 helper CD4+ cells, memory CD8+ cells, and cells that belong to the innate immune system, such as natural killer cells, eosinophils, and neutrophils. In addition, local inflammation associated with rejection also is tightly regulated at the graft level by regulatory T cells and mast cells.

A vascular graft is a tissue used to patch or replace injured or diseased areas of arteries or to construct a new vessel for hemodialysis access in a dialysis patient. While the success rate of vascular grafts with a large diameter, e.g., greater than about 6 mm, has risen steadily, the success rate of smaller vascular grafts has been hampered by the development of intimal hyperplasia, and ultimately atherosclerosis, which gradually reduces blood flow, leading to retrograde thrombosis and failure. Moreover, mechanical factors, including, but not limited to, disturbed flow at the anastomosis where blood vessels are connected leading to fluctuations in shear stress at the endothelium, injury due to suturing, and stress concentration at the anastomosis, also have been shown to be important in the genesis of intimal hyperplasia. Additionally, vascular graft failure may be attributed to hematoma development, infection, collection of fluid, and an inappropriate vascular bed (meaning the intricate network of minute blood vessels that ramifies through the tissues of the body or of one of its parts).

Bypass surgery is a procedure by which a surgeon creates a new tubular pathway for the movement of fluids and/or other substances in the body. For example, coronary artery bypass graft (CABG) surgery is a major cardiovascular procedure in which vessels harvested from the patient are used to re-route blood around blocked coronary arteries, allowing blood to flow through the newly-implanted vessels to the heart. Similarly, peripheral arterial bypass graft surgery also is performed to re-route blood around blocked arteries in the leg, preventing leg pain (claudication) and limb loss. In addition, in order to facilitate access to the blood flow of a dialysis patient, a hemodialysis access surgery that connects an artery to a vein using a synthetic tube, or graft (hemodialysis access graft) often is performed. The most common source of grafts, for example, for peripheral and CABG surgery is human greater saphenous vein (HSV) harvested from the patient's leg.

Recent clinical trials have confirmed that CABG is the best option for patients with severe coronary heart disease who cannot be managed effectively with drug therapy or who cannot be as effectively treated with less-invasive interventional procedures, such as angioplasty or stenting. According to a report, there are over 1 million CABG procedures performed annually worldwide and 480,000 in the United States. While there is no canonical annual cost figure for the CABG surgery, minimum health care expenditures are estimated at over $1.5 billion/year. Moreover, CABG patients typically are older, have Stage 3 and 4 disease, and—due to their cardiovascular conditions—have high rates of mortality and complications.

Bypass operations are expensive procedures (typically >$30,000), and thus failure of implanted grafts subsequently requires additional expensive procedures. Despite this fact, less than half of these grafts remain patent after 12 years, with graft failure leading to myocardial infarction, limb loss and death. Thus, approaches to decrease vascular graft failure rates would improve arterial bypass procedure outcomes and decrease graft failure-associated costs.

Among the reasons described above, however, two adverse reactions—vasospasm and intimal hyperplasia—are, at present, the leading causes of vascular graft failure.

Vasospasm as used herein refers to an involuntary contraction of vascular smooth muscle cells that can acutely reduce blood supply and tissue oxygenation. During surgery, a surgeon mechanically dilates vessels in order to break vessel spasm, which is refractory to current vasodilator pharmacologic approaches. Such mechanical dilation, however, appears to reduce functional contractility of the vessel smooth muscle cells and to decrease ultimate viability of the cells.

The graft procedure also triggers inflammatory and fibrotic reactions in the host, leading to a disorder called intimal hyperplasia. Intimal hyperplasia is the thickening of the tunica intima (the innermost layer of an artery or vein) of a blood vessel as a complication of a reconstruction procedure or endarterectomy (the surgical stripping of a fat-encrusted, thickened arterial lining so as to open or widen the artery for improved blood circulation) and is considered a leading cause of graft failure.

3. Inflammation

The classic signs of inflammation are pain (dolor), heat (calor), redness (rubor), swelling (tumor), and loss of function (functio laesa). Histologically, inflammation involves a complex series of events, including dilatation of arterioles, capillaries, and venules, with increased permeability and blood flow; exudation of fluids, including plasma proteins; and leukocytic migration into the inflammatory focus.

Regardless of the initiating agent, the physiologic changes accompanying acute inflammation encompass four main features: (1) vasodilation, which results in a net increase in blood flow, which is one of the earliest physical responses to acute tissue injury; (2) contraction of endothelial cells lining the venules in response to inflammatory stimuli, which widens the intracellular junctions to produce gaps, leading to increased vascular permeability and thereby permitting leakage of plasma proteins and blood cells out of blood vessels; (3) inflammation characterized by a strong infiltration of leukocytes at the site of inflammation, particularly neutrophils (polymorphonuclear cells), which promote tissue damage by releasing toxic substances at the vascular wall or in uninjured tissue; and (4) fever, produced by pyrogens released from leukocytes in response to specific stimuli.

During the inflammatory process, soluble inflammatory mediators of the inflammatory response work together with cellular components in a systemic fashion in the attempt to contain and eliminate the agents causing physical distress.

Several disorders associated with inflammation underlie a variety of diseases. These include, but are not limited to, chronic inflammation, reperfusion injury, and vasculitis.

Chronic inflammation is a pathological condition characterized by concurrent active inflammation, tissue destruction, and attempts at repair by the infiltration of mononuclear immune cells (monocytes, macrophages, lymphocytes, and plasma cells), tissue destruction, and attempts at healing (angiogenesis and fibrosis). Endogenous causes include persistent acute inflammation. Exogenous causes are varied and include bacterial infection, prolonged exposure to chemical agents, and autoimmune reactions. In acute inflammation, removal of the stimulus halts the recruitment of monocytes (which become macrophages under appropriate activation) into the inflamed tissue, and existing macrophages exit the tissue via lymphatics. In chronically inflamed tissue, the stimulus is persistent, and therefore recruitment of monocytes is maintained, existing macrophages are tethered in place, and proliferation of macrophages is stimulated.

The term “reperfusion injury” refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function. Symptoms include, but are not limited to, elevated white blood cell levels, apoptosis, and free radical accumulation.

Vasculitis refers to a disorder characterized by inflammatory destruction of blood vessels (arteries and veins). Symptoms of vasculitis usually are systemic with single or multiorgan dysfunction. These symptoms may include fatigue, weakness, fever, arthralgias, abdominal pain, hypertension, renal insufficiency, and neurologic dysfunction. Additional symptoms may include mononeuritis multiplex, palpable purpura and pulmonary-renal syndrome.

4. Fibrosis

The graft procedure also may trigger fibrosis, meaning the formation or development of excess fibrous connective tissue in an organ or tissue as a reparative or reactive process, as opposed to a formation of fibrous tissue as a normal constituent of an organ or tissue, which also may lead to vascular graft failure through formation of atherosclerotic plaques in the grafted arteries or veins, which progress to thrombosis. Such atherosclerotic plaques contain large amounts of newly proliferated fibrous tissues, lipid-engorged macrophages, and calcified constituents.

While fibrosis in other tissues generally involves activation of fibroblasts, vessel injury induces smooth muscle cells to proliferate and deposit extracellular matrix proteins, such as collagen and elastin fibers, into the inner layer of the vessels. The combined effects of smooth muscle cell activation, lipid deposition, and excessive fibrous tissue formation encroaches on the luminal space of the involved vessel, and may eventually restrict blood flow, leading to vascular graft failure.

5. Cellular and Molecular Events Associated with Intimal Hyperplasia

At the cellular level, intimal hyperplasia is mediated by a sequence of events, including inflammatory processes in response to vessel trauma, vascular smooth muscle cell proliferation, vascular smooth muscle cell migration, and extracellular matrix production. These events are associated with a phenotypic modulation of smooth muscle cells from a contractile to a synthetic phenotype, with “synthetic” cells secreting extracellular matrix proteins, leading to pathologic narrowing of the vessel lumen, graft stenosis and ultimately graft failure.

Molecularly, a vascular graft procedure has been shown to activate the p38MAPK pathway that leads to activation of two distinct downstream signaling pathways—the p38MAPK-MK2-HSPB1 pathway and the inflammatory cytokine biosynthesis pathway. The activated p38MAPK-MK2-HSPB1 pathway, in turn, switches smooth muscle cells into a synthetic mode to produce extracellular matrix proteins, whereas the activated inflammatory cytokine biosynthesis pathway increases the level of inflammatory cytokines by stabilizing their mRNAs, leading to inflammation-induced intimal hyperplasia.

6. Vascular Grafts and Activation of p38MAPK-MK2 Pathway

As described above, at the molecular level, a vascular graft procedure activates the p38 MAPK pathway and its downstream target MK2. The activated MK2, in turn, activates HSPB1 kinase via phosphorylation, and thereby enhances vascular smooth muscle cell proliferation, vascular smooth muscle cell migration, and production of extracellular matrix proteins. On the other hand, the activated MK2 kinase also stabilizes inflammatory cytokine mRNAs, including TNF-α, IL-1 and IL-6, leading directly to inflammatory-induced intimal hyperplasia.

Since the activation of the p38 MAPK-MK2-HSPB1 pathway and the inflammatory cytokine biosynthesis pathway lead to pathologic narrowing of the vessel lumen, graft stenosis, and ultimate graft failure, it is imperative to develop a therapeutic that targets at the level of MK2, which can suppress both the p38MAPK-MK2-HSPB1 pathway and the inflammatory cytokine biosynthesis pathway, in order to treat or prevent complications associated with vascular graft surgery and to ensure patency of the transplanted vascular grafts.

While some inhibitors targeting the downstream signaling molecules of TGF-β pathway, e.g., TGF-β (Justiva™, Renovo), p38MAPK, or HSPB1, have been developed previously, they are not likely to be effective to combat adverse reactions associated with vascular graft procedures. For example, TGF-β, an extracellular ligand in the TGF-β pathway, and p38MAPK, an upstream kinase in the TGF-β pathway, both can affect diverse downstream signaling pathways that regulate essential cellular functions, e.g., normal wound healing. Accordingly, inhibition of upstream signaling molecules in the TGF-β pathway, such as TGF-β or p38MAPK, can induce toxicity and interfere with essential functions of the cells. Furthermore, whereas HSPB1 inhibitors may combat adverse changes in the properties of smooth muscle cells, they are not effective in suppressing inflammation-induced intimal hyperplasia.

7. Pre-Atherosclerotic Intimal Hyperplasia

In addition to its involvement in vascular graft failure, intimal hyperplasia also is considered a precursor lesion for some atherosclerosis in humans.

Arteriosclerosis (hardening of the arteries) is characterized by smooth muscle cell hyperplasia or hypertrophy and matrix accumulation in the tunica intima and/or tunica media with or without lipid deposition, resulting in thickening and stiffness of the arterial wall. Arteriosclerosis includes spontaneous arteriosclerosis, accelerated arteriosclerosis (e.g., transplant arteriosclerosis), restenosis (re-narrowing of artery after balloon angioplasty, a surgical procedure involving inflating a small balloon inside a narrowed blood vessel to stretch out the vessel), and vein graft atherosclerosis.

Atherosclerosis, the most common form of arteriosclerosis, is a complex process that begins with the appearance of cholesterol-laden macrophages (foam cells) in the intima of an artery. In response to the appearance of cholesterol-laden macrophages, vascular smooth muscle cells proliferate under the influence of platelet factors. The responding smooth muscle cells show altered lipid metabolism, altered growth factor production, altered extracellular matrix production, smaller size, fewer intracellular junctions, and the presence of fatty vacuoles. As a result, a plaque consisting of smooth muscle cells and leukocytes forms at the site, which causes further deposition of lipid, leading to formation of fibrotic and calcified tissue. The expanded atherosclerotic plaque gradually obstructs the artery and induces ischemic injuries to the vessel, causing more acute and severer impairment of blood flow, a principal mechanism that causes coronary artery diseases (including, e.g., arteriosclerotic heart disease, myocardial infarction), peripheral vascular diseases, and stroke (such as cerebral infarction).

When atherosclerosis develops, advanced lesions form first in some regions with adaptive intimal thickening. For example, pre-atherosclerotic intimal hyperplasia develops universally within the first decade in the atherosclerosis-prone coronary arteries, e.g., the post-branch region of the proximal left anterior descending coronary artery, and such lesions are composed predominantly of smooth muscle cells in a proteoglycan-rich matrix with small numbers of macrophages. Therefore, preventing pre-atherosclerotic intimal hyperplasia at an early stages of atherosclerosis development could be an effective treatment for some cases of atherosclerosis in humans.

A new approach to inhibit both activation of the p38MAPK-MK2-HSPB1 pathway and the biosynthesis of inflammatory cytokines would allow preservation of functional contractility and viability of transplanted vascular grafts and minimize development of intimal hyperplasia, both of which lead to vascular graft failure. Such an inhibitor also would be useful in preventing or treating pre-atherosclerotic intimal hyperplasia, which is responsible for some cases of atherosclerosis. The described invention offers such an approach.

### SUMMARY

According to one aspect, the described invention provides a method for treating failure of a vascular graft in a subject in need of such treatment, the method comprising administering a therapeutically effective amount of a pharmaceutical composition comprising a polypeptide of amino sequence YARAAARQARAKALARQLGVAA (SEQ ID NO: 1) or a functional equivalent thereof, and a pharmaceutically acceptable carrier. According to one embodiment of the method, the step of administering is by implanting a biomedical device, wherein the device is a vascular graft, and wherein the composition is disposed on or in the graft. According to another embodiment, the step of administering occurs parenterally. According to another embodiment, the step of administering occurs topically. According to another embodiment, the vascular graft is an autologous graft. According to another embodiment, the vascular graft is a syngeneic graft. According to another embodiment, the vascular graft is an allogeneic graft. According to another embodiment, the vascular graft is a xenograft. According to another embodiment, the vascular graft is a synthetic graft. According to another embodiment, the vascular graft is a prosthetic graft. According to another embodiment, the vascular graft is a tissue engineered graft. According to another embodiment, the vascular graft is a vascular access graft. According to another embodiment, the vascular graft is an arteriovenous graft. According to another embodiment, the vascular graft is a coronary artery bypass graft. According to another embodiment, the step of administering occurs at one time as a single dose, wherein the one time is during vascular graft surgery. According to another embodiment, the step of administering is performed as a plurality of doses over a period of time. According to another embodiment, the period of time is a day, a week, a month, a year, or multiples thereof. According to another embodiment, the step of administering is performed at least once daily. According to another embodiment, the step of administering is performed at least once daily for a period of at least one week. According to another embodiment, the step of administering is performed at least once weekly. According to another embodiment, the step of administering is performed weekly for a period of at least one month. According to another embodiment, the step of administering is performed at least once monthly. According to another embodiment, the method reduces stenosis of the vascular graft. According to another embodiment, the method reduces vasospasm of at least one blood vessel related to the vascular graft. According to another embodiment, the method reduces intimal hyperplasia of at least one blood vessel related to the vascular graft. According to another embodiment, the functional equivalent of the polypeptide YARAAARQARAKALARQLGVAA (SEQ ID NO: 1) has a substantial sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 70 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 80 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 90 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 95 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) is a polypeptide of amino acid sequence WLRRIKAWLRRIKALNRQLGVAA (SEQ ID NO: 3). According to another embodiment, the functional equivalent of the polypeptide YARAAARQARAKALARQLGVAA (SEQ ID NO: 1) is a polypeptide of amino acid sequence FAKLAARLYRKALARQLGVAA (SEQ ID NO: 4). According to another embodiment, the functional equivalent of the polypeptide YARAAARQARAKALARQLGVAA (SEQ ID NO: 1) is a polypeptide of amino acid sequence KAFAKLAARLYRKALARQLGVAA (SEQ ID NO: 5). According to another embodiment, the functional equivalent of the polypeptide YARAAARQARAKALARQLGVAA (SEQ ID NO: 1) is a polypeptide of amino acid sequence YARAAARQARAKALNRQLGVAA (SEQ ID NO: 6). According to another embodiment, the functional equivalent of the polypeptide YARAAARQARAKALARQLGVAA (SEQ ID NO: 1) is a polypeptide of amino acid sequence YARAAARQARAKALARQLAVA (SEQ ID NO: 7). According to another embodiment, the functional equivalent of the polypeptide YARAAARQARAKALARQLGVAA (SEQ ID NO: 1) is a polypeptide of amino acid sequence YARAAARQARAKALARQLGVA (SEQ ID NO: 8). According to another embodiment, the functional equivalent of the polypeptide YARAAARQARAKALARQLGVAA (SEQ ID NO: 1) is a polypeptide of amino acid sequence YARAAARQARAKALNRQLAVA (SEQ ID NO: 9). According to another embodiment, the functional equivalent of the polypeptide YARAAARQARAKALARQLGVAA (SEQ ID NO: 1) is a polypeptide of amino acid sequence YARAAARQARAKALNRQLGVA (SEQ ID NO: 10). According to another embodiment, the functional equivalent of the polypeptide YARAAARQARAKALARQLGVAA (SEQ ID NO: 1) is a polypeptide of amino acid sequence YARAAARQARAKALNRQLGVAA (SEQ ID NO: 11). According to another embodiment, the functional equivalent of the polypeptide YARAAARQARAKALARQLGVAA (SEQ ID NO: 1) is a polypeptide of amino acid sequence YARAAARQARAKALNRQLAVAA (SEQ ID NO: 12). According to another embodiment, the functional equivalent of the polypeptide YARAAARQARAKALARQLGVAA (SEQ ID NO: 1) is a fusion protein comprising a first polypeptide operatively linked to a second polypeptide, wherein the first polypeptide is of amino acid sequence YARAAARQARA (SEQ ID NO: 26), and wherein the second polypeptide comprises a therapeutic domain that has a substantial identity to amino acid sequence KALARQLGVAA (SEQ ID NO: 2). According to another embodiment, the second polypeptide has at least 70 percent sequence identity to amino acid sequence KALARQLGVAA (SEQ ID NO: 2). According to another embodiment, the second polypeptide has at least 80 percent sequence identity to amino acid sequence KALARQLGVAA (SEQ ID NO: 2). According to another embodiment, the second polypeptide has at least 90 percent sequence identity to amino acid sequence KALARQLGVAA (SEQ ID NO: 2). According to another embodiment, the second polypeptide has at least 95 percent sequence identity to amino acid sequence KALARQLGVAA (SEQ ID NO: 2). According to another embodiment, the second polypeptide is of amino acid sequence KALARQLAVA (SEQ ID NO: 13). According to another embodiment, the second polypeptide is of amino acid sequence KALARQLGVA (SEQ ID NO: 14). According to another embodiment, the second polypeptide is of amino acid sequence KALARQLGVAA (SEQ ID NO: 15). According to another embodiment, the second polypeptide is of amino acid sequence KALNRQLGVAA (SEQ ID NO: 16). According to another embodiment, the second polypeptide is of the amino acid sequence KAANRQLGVAA (SEQ ID NO: 17). According to another embodiment, the second polypeptide is of amino acid sequence KALNAQLGVAA (SEQ ID NO: 18). According to another embodiment, the second polypeptide is of amino acid sequence KALNRALGVAA (SEQ ID NO: 19). According to another embodiment, the second polypeptide is of amino acid sequence KALNRQAGVAA (SEQ ID NO: 20). According to another embodiment, the second polypeptide is of amino acid sequence KALNRQLAVA (SEQ ID NO: 21). According to another embodiment, the second polypeptide is of amino acid sequence KALNRQLAVAA (SEQ ID NO: 22). According to another embodiment, the second polypeptide is of amino acid sequence KALNRQLGAAA (SEQ ID NO: 23). According to another embodiment, the second polypeptide is of amino acid sequence KALNRQLGVA (SEQ ID NO: 24). According to another embodiment, the second polypeptide is of amino acid sequence KKKALNRQLGVAA (SEQ ID NO: 25). According to another embodiment, the functional equivalent of the polypeptide YARAAARQARAKALARQLGVAA (SEQ ID NO: 1) is a polypeptide is a fusion protein comprising a first polypeptide operatively linked to a second polypeptide, wherein the first polypeptide comprises a protein transduction domain functionally equivalent to amino acid sequence YARAAARQARA (SEQ ID NO: 26), and wherein the second polypeptide is of amino acid sequence KALARQLGVAA (SEQ ID NO: 2). According to another embodiment, the first polypeptide is of amino acid sequence WLRRIKAWLRRIKA (SEQ ID NO: 27). According to another embodiment, the first polypeptide is of amino acid sequence WLRRIKA (SEQ ID NO: 28). According to another embodiment, the first polypeptide is of amino acid sequence YGRKKRRQRRR (SEQ ID NO: 29). According to another embodiment, the first polypeptide is of amino acid sequence WLRRIKAWLRRI (SEQ ID NO: 30). According to another embodiment, the first polypeptide is of amino acid sequence FAKLAARLYR (SEQ ID NO: 31). According to another embodiment, the first polypeptide is of amino acid sequence KAFAKLAARLYR (SEQ ID NO: 32).

According to another aspect, the described invention provides a method for treating a vascular disease comprising intimal hyperplasia in a subject in need of such treatment, the method comprising administering a therapeutically effective amount of a pharmaceutical composition comprising a polypeptide of amino acid sequence YARAAARQARAKALARQLGVAA (SEQ ID NO: 1) or a functional equivalent thereof, and a pharmaceutically acceptable carrier. According to one embodiment of the method, the vascular disease is a pre-atherosclerotic intimal hyperplasia. According to another embodiment, the vascular disease is an atherosclerosis. According to another embodiment, the step of administering is by implanting a biomedical device, wherein the pharmaceutical composition is disposed on or in the device. According to another embodiment, the step of administering occurs parenterally. According to another embodiment, the step of administering occurs topically. According to another embodiment, the step of administering occurs at one time as single dose, wherein the one time is during vascular graft surgery. According to another embodiment, the step of administering is performed as a plurality of doses over a period of time. According to another embodiment, the period of time is a day, a week, a month, a year, or multiples thereof. According to another embodiment, the administering is performed at least once daily. According to another embodiment, the step of administering is performed at least once daily for a period of at least one week. According to another embodiment, the step of administering is performed at least once weekly. According to another embodiment, the step of administering is performed weekly for a period of at least one month. According to another embodiment, the step of administering is performed at least once monthly. According to another embodiment, the functional equivalent of the polypeptide YARAAARQARAKALARQLGVAA (SEQ ID NO: 1) has a substantial sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 70 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 80 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 90 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 95 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) is of amino acid sequence WLRRIKAWLRRIKALNRQLGVAA (SEQ ID NO: 3). According to another embodiment, the functional equivalent of the polypeptide YARAAARQARAKALARQLGVAA (SEQ ID NO: 1) is of amino acid sequence FAKLAARLYRKALARQLGVAA (SEQ ID NO: 4). According to another embodiment, the functional equivalent of the polypeptide YARAAARQARAKALARQLGVAA (SEQ ID NO: 1) is of amino acid sequence KAFAKLAARLYRKALARQLGVAA (SEQ ID NO: 5). According to another embodiment, the functional equivalent of the polypeptide YARAAARQARAKALARQLGVAA (SEQ ID NO: 1) is of amino acid sequence YARAAARQARAKALNRQLGVAA (SEQ ID NO: 6). According to another embodiment, the functional equivalent of the polypeptide YARAAARQARAKALARQLGVAA (SEQ ID NO: 1) is of amino acid sequence YARAAARQARAKALARQLAVA (SEQ ID NO: 7). According to another embodiment, the functional equivalent of the polypeptide YARAAARQARAKALARQLGVAA (SEQ ID NO: 1) is of amino acid sequence YARAAARQARAKALARQLGVA (SEQ ID NO: 8). According to another embodiment, the functional equivalent of the polypeptide YARAAARQARAKALARQLGVAA (SEQ ID NO: 1) is of amino acid sequence YARAAARQARAKALNRQLAVA (SEQ ID NO: 9). According to another embodiment, the functional equivalent of the polypeptide YARAAARQARAKALARQLGVAA (SEQ ID NO: 1) is of amino acid sequence YARAAARQARAKALNRQLGVA (SEQ ID NO: 10). According to another embodiment, the functional equivalent of the polypeptide YARAAARQARAKALARQLGVAA (SEQ ID NO: 1) is of amino acid sequence YARAAARQARAKALNRQLGVAA (SEQ ID NO: 11). According to another embodiment, the functional equivalent of the polypeptide YARAAARQARAKALARQLGVAA (SEQ ID NO: 1) is of amino acid sequence YARAAARQARAKALNRQLAVAA (SEQ ID NO: 12). According to another embodiment, the functional equivalent of the polypeptide YARAAARQARAKALARQLGVAA (SEQ ID NO: 1) is a fusion protein comprising a first polypeptide operatively linked to a second polypeptide, wherein the first polypeptide is of amino acid sequence YARAAARQARA (SEQ ID NO: 26), and wherein the second polypeptide comprises a therapeutic domain that has a substantial identity to amino acid sequence KALARQLGVAA (SEQ ID NO: 2). According to another embodiment, the second polypeptide has at least 70 percent sequence identity to KALARQLGVAA (SEQ ID NO: 2). According to another embodiment, the second polypeptide has at least 80 percent sequence identity to KALARQLGVAA (SEQ ID NO: 2). According to another embodiment, the second polypeptide has at least 90 percent sequence identity to KALARQLGVAA (SEQ ID NO: 2). According to another embodiment, the second polypeptide has at least 95 percent sequence identity to KALARQLGVAA (SEQ ID NO: 2). According to another embodiment, the second polypeptide is of amino acid sequence KALARQLAVA (SEQ ID NO: 13). According to another embodiment, the second polypeptide is of amino acid sequence KALARQLGVA (SEQ ID NO: 14). According to another embodiment, the second polypeptide is of amino acid sequence KALARQLGVAA (SEQ ID NO: 15). According to another embodiment, the second polypeptide is of amino acid sequence KALNRQLGVAA (SEQ ID NO: 16). According to another embodiment, the second polypeptide is of amino acid sequence KAANRQLGVAA (SEQ ID NO: 17). According to another embodiment, the second polypeptide is of amino acid sequence KALNAQLGVAA (SEQ ID NO: 18). According to another embodiment, the second polypeptide is of amino acid sequence KALNRALGVAA (SEQ ID NO: 19). According to another embodiment, the second polypeptide is of amino acid sequence KALNRQAGVAA (SEQ ID NO: 20). According to another embodiment, the second polypeptide is of amino acid sequence KALNRQLAVA (SEQ ID NO: 21). According to another embodiment, the second polypeptide is of amino acid sequence KALNRQLAVAA (SEQ ID NO: 22). According to another embodiment, the second polypeptide is of amino acid sequence KALNRQLGAAA (SEQ ID NO: 23). According to another embodiment, the second polypeptide is of amino acid sequence KALNRQLGVA (SEQ ID NO: 24). According to another embodiment, the second polypeptide is of amino acid sequence KKKALNRQLGVAA (SEQ ID NO: 25). According to another embodiment, the functional equivalent of the polypeptide YARAAARQARAKALARQLGVAA (SEQ ID NO: 1) is a fusion protein comprising a first polypeptide operatively linked to a second polypeptide, wherein the first polypeptide comprises a protein transduction domain functionally equivalent to amino acid sequence YARAAARQARA (SEQ ID NO: 26), and wherein the second polypeptide is of amino acid sequence KALARQLGVAA (SEQ ID NO: 2). According to another embodiment, the first polypeptide is of amino acid sequence WLRRIKAWLRRIKA (SEQ ID NO: 27). According to another embodiment, the first polypeptide is of amino acid sequence WLRRIKA (SEQ ID NO: 28). According to another embodiment, the first polypeptide is of amino acid sequence YGRKKRRQRRR (SEQ ID NO: 29). According to another embodiment, the first polypeptide is of amino acid sequence WLRRIKAWLRRI (SEQ ID NO: 30). According to another embodiment, the first polypeptide is of amino acid sequence FAKLAARLYR (SEQ ID NO: 31). According to another embodiment, the first polypeptide is of amino acid sequence KAFAKLAARLYR (SEQ ID NO: 32).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of a transforming growth factor-beta (TGF-β) signaling pathway and a tumor necrosis factor-alpha (TNF-α) signaling pathway relevant to MK2 phosphorylation.

FIG. 2 shows the effect of pharmacological doses of MMI-0100 (YARAAARQARAKALARQLGVAA; SEQ ID NO: 1) on human endothelial cell (EC) (A) and smooth muscle cell (SMC) proliferation (B). FIG. 2C shows phase contrast images of ECs and SMCs treated with MMI-0100 (YARAAARQARAKALARQLGVAA; SEQ ID NO: 1) for 24 hours.

FIG. 3 shows dose-dependent inhibition of tumor necrosis factor-alpha (TNF-α) and interleukin-1 beta (IL-1β) expression by MMI-0100 (YARAAARQARAKALARQLGVAA; SEQ ID NO: 1) in human monocytic leukemia cells (THP-1) treated with phorbol 12-myristate 13-acetate (PMA) and lipopolysaccharide (LPS).

FIG. 4 shows that MMI-0100 (YARAAARQARAKALARQLGVAA; SEQ ID NO: 1; labeled as “YARA”) inhibits production of inflammatory cytokines triggered by TNF-α in vitro.

FIG. 5 shows the anti-inflammatory effect of pharmacological doses of MMI-0100 (YARAAARQARAKALARQLGVAA; SEQ ID NO: 1) in endothelial cells. MMI-0100 (YARAAARQARAKALARQLGVAA; SEQ ID NO: 1) treatment reduced the level of TNF-α-induced IL-6 expression to that of the untreated control (A) but did not affect the level of TNF-α-induced IL-8 expression (B).

FIG. 6. The upper panels show representative tracings of isometric contractions of human saphenous veins (HSV) harvested from two patients (HSV54 and HSV 55). The lower panels show cellular viability data for patients HSV54 and HSV55.

FIG. 7 shows that the MK2 inhibitor of the described invention YARAAARQARAKALARQLGVAA (MMI-0100; SEQ ID NO: 1) enhances sodium nitroprusside-induced relaxation of human saphenous vein.

FIG. 8 shows that MMI-0100 (YARAAARQARAKALARQLGVAA; SEQ ID NO: 1) treatment enhances relaxation of human saphenous veins (HSV).

FIG. 9 shows the effect of MMI-0100 (YARAAARQARAKALARQLGVAA; SEQ ID NO: 1) or rHSPB1 (SEQ ID NO: 36) treatment on the thickness of the intimal layer.

FIG. 10 shows that MMI-0100 (YARAAARQARAKALARQLGVAA; SEQ ID NO: 1) treatment reduces intimal hyperplasia in a human saphenous vein organ culture model.

FIG. 11 shows representative micrographs of human saphenous veins (HSV) either untreated or treated with 10 μM, 50 μM, or 100 μM of MMI-0100 (YARAAARQARAKALARQLGVAA; SEQ ID NO: 1).

FIG. 12 shows in vivo evaluation of the effect of MMI-0100 (YARAAARQARAKALARQLGVAA; SEQ ID NO: 1; labeled as “MK2i”) in a mouse vein graft model (measurement of vein wall thickness over 28 days in vivo).

FIG. 13 shows in vivo evaluation of the effect of MMI-0100 (YARAAARQARAKALARQLGVAA; SEQ ID NO: 1) in a mouse vein graft model (vein wall histology after 28 days in vivo)

FIG. 14 shows the effect of MMI-0100 (YARAAARQARAKALARQLGVAA; SEQ ID NO: 1) in an in vivo model of intimal hyperplasia (a mouse model of vein graft adaptation): (A) representative ultrasound images of vein grafts at 4 weeks post treatment with phosphate-buffered saline (PBS) or MMI-0100 (YARAAARQARAKALARQLGVAA; SEQ ID NO: 1); (B) analysis of vein grafts wall thickness by ultrasound at each week for 4 weeks following treatment with PBS or MMI-0100 (YARAAARQARAKALARQLGVAA; SEQ ID NO: 1); (C) representative histochemical images of vein grafts stained with hematoxylin & eosin (H&E) at 4 weeks post treatment with PBS or MMI-0100 (YARAAARQARAKALARQLGVAA; SEQ ID NO: 1); (D) analysis of vein graft wall thickness by histochemistry at 4 weeks post treatment with PBS or MMI-0100 (YARAAARQARAKALARQLGVAA; SEQ ID NO: 1); (E-F) analysis of vein grafts for F4/80 (a macrophage marker) immunohistochemical reactivity at 4 weeks post treatment with PBS or MMI-0100 (YARAAARQARAKALARQLGVAA; SEQ ID NO: 1).

FIG. 15 shows the effect of physiological doses of MMI-0100 (YARAAARQARAKALARQLGVAA; SEQ ID NO: 1) on murine endothelial cells (EC): (A) western blot analysis of murine EC lysates for expression of Eph-B4, a marker of venous identity; (B) the effect of physiological doses of MMI-0100 (YARAAARQARAKALARQLGVAA; SEQ ID NO: 1) on EC proliferation; (C) the effect of physiological doses of MMI-0100 (YARAAARQARAKALARQLGVAA; SEQ ID NO: 1) on EC apoptosis; (D) the effect of physiological doses of MMI-0100 (YARAAARQARAKALARQLGVAA; SEQ ID NO: 1) on monocyte chemotactic protein-1 (MCP-1) production; and (E) the effect of physiological doses of MMI-0100 (YARAAARQARAKALARQLGVAA; SEQ ID NO: 1) on nitric oxide (NO) production.

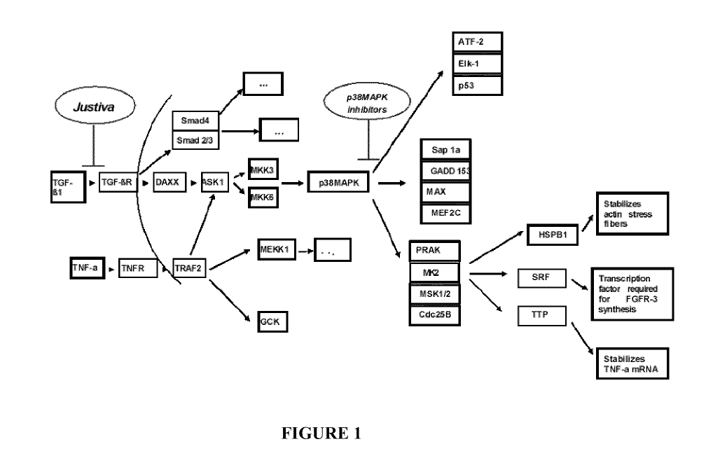

### DETAILED DESCRIPTION OF THE INVENTION

The described invention provides pharmaceutical compositions and methods for treating or preventing vascular graft failure in a subject in need of such treatment, the method comprising administering a therapeutically effective amount of a pharmaceutical composition comprising a polypeptide of amino acid sequence YARAAARQARAKALARQLGVAA (SEQ ID NO: 1), or a functional equivalent thereof, and a pharmaceutically acceptable carrier. The methods also are useful for treating a pre-atherosclerotic intimal hyperplasia condition in a subject in need of such treatment, by administering a therapeutically effective amount of the pharmaceutical composition.

### GLOSSARY

The terms “amino acid residue” or “amino acid” or “residue” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids. The amino acids may be L- or D-amino acids. An amino acid may be replaced by a synthetic amino acid, which is altered so as to increase the half-life of the peptide, increase the potency of the peptide, or increase the bioavailability of the peptide.

The single letter designation for amino acids is used predominately herein. As is well known by one of skill in the art, such single letter designations are as follows:

A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is glutamine; R is arginine; S is serine; T is threonine; V is valine; W is tryptophan; and Y is tyrosine.

The following represent groups of amino acids that are conservative substitutions for one another:

1) Alanine (A), Serine (S), Threonine (T);

2) Aspartic Acid (D), Glutamic Acid (E);

3) Asparagine (N), Glutamic Acid (Q);

4) Arginine (R), Lysine (K);

5) Isoleucine (I), Leucine (L), Methionine (M), Valine (V); and

6) Phenylalanine (F), Tyrosine (Y), Tryptophan (W).

As used herein, the singular forms “a”, “an” and “the” include plural referents unless the context clearly dictates otherwise. For example, reference to a “polypeptide” means one or more polypeptides.

The term “addition” as used herein refers to the insertion of one or more bases, or of one or more amino acids, into a sequence.

The term “administer” as used herein refers to dispensing, supplying, applying, giving, apportioning or contributing. The terms “administering” or “administration” are used interchangeably and include in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing the conventional nontoxic pharmaceutically acceptable carriers, adjuvants, and vehicles as desired, or may be locally administered by means such as, but not limited to, injection, implantation, grafting, topical application, or parenterally. Additional administration may be performed, for example, intravenously, pericardially, orally, via implant, transmucosally, transdermally, topically, intramuscularly, subcutaneously, intraperitoneally, intrathecally, intralymphatically, intralesionally, or epidurally. Administering can be performed, for example, once, a plurality of times, and/or over one or more extended periods.

The term “atherosclerosis” as used herein refers to a condition in which plaque builds up inside the arteries. The plaque is made up of fat, cholesterol, calcium and other substances found in the blood. Over time, plaque hardens and narrows the arteries, limiting the flow of oxygen-rich blood to organs and other parts of the body, which lead to serious problems, such as heart attack or stroke. Atherosclerotic plaques also contain large amounts of fibrous tissue composed of smooth muscle cells, a condition known as fibroproliferative (FP) response. FP response is believed to be a defensive, protective, physiologic response to injury designed to wall off, contain, enclose, or sequester the injurious agent, and then to assist in resolution of the injury. Plaque tissue is produced primarily by intimal smooth muscle cells, and not by fibroblasts, the usual cell type normally involved in would repair.

The term “autologous graft” or “autograft” as used herein refers to a tissue that is grafted into a new position in or on the body of the same individual.

The terms “carrier” and “pharmaceutical carrier” as used herein refer to a pharmaceutically acceptable inert agent or vehicle for delivering one or more active agents to a subject, and often is referred to as “excipient.” The (pharmaceutical) carrier must be of sufficiently high purity and of sufficiently low toxicity to render it suitable for administration to the subject being treated. The (pharmaceutical) carrier further should maintain the stability and bioavailability of an active agent, e.g., a polypeptide of the described invention. The (pharmaceutical) carrier can be liquid or solid and is selected, with the planned manner of administration in mind, to provide for the desired bulk, consistency, etc., when combined with an active agent and other components of a given composition. The (pharmaceutical) carrier may be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulphate, etc.). Other suitable (pharmaceutical) carriers for the compositions of the described invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like. Compositions that are for parenteral administration of a polypeptide of the described invention may include (pharmaceutical) carriers such as sterile aqueous solutions, non-aqueous solutions in common solvents such as alcohols, or solutions of the polypeptide in a liquid oil base.

The term “condition” as used herein refers to a variety of health states and is meant to include disorders or diseases caused by any underlying mechanism or disorder, injury, and the promotion of healthy tissues and organs. Disorders may include, for example, but are not limited to, inflammatory diseases, fibrosis, endotoxic shock, localized inflammatory disease, atherosclerotic cardiovascular disease, Alzheimer's disease, oncological diseases, neural ischemia, rheumatoid arthritis, Crohn's disease, inflammatory bowel disease, intimal hyperplasia, stenosis, restenosis, atherosclerosis, smooth muscle cell tumors and metastasis, smooth muscle spasm, angina, Prinzmetal's angina, ischemia, stroke, bradycardia, hypertension, cardiac hypertrophy, renal failure, stroke, pulmonary hypertension, asthma, toxemia of pregnancy, pre-term labor, pre-eclampsia, eclampsia, Raynaud's disease or phenomenon, hemolytic-uremia, anal fissure, achalasia, impotence, migraine, ischemic muscle injury associated with smooth muscle spasm, vasculopathy, bradyarrythmia, congestive heart failure, stunned myocardium, pulmonary hypertension, diastolic dysfunction, gliosis (proliferation of astrocytes, and may include deposition of extracellular matrix (ECM) deposition in damaged areas of the central nervous system), chronic obstructive pulmonary disease (i.e, respiratory tract diseases characterized by airflow obstruction or limitation; includes but is not limited to chronic bronchitis and emphysema), osteopenia, endothelial dysfunction, inflammation, degenerative arthritis, anklyosing spondylitis, Sjorgen's disease, Guilliamé-Barre disease, infectious disease, sepsis, endotoxemic shock, psoriasis, radiation enteritis, scleroderma, cirrhosis, interstitial fibrosis, colitis, appendicitis, gastritis, laryngitis, meningitis, pancreatitis, otitis, reperfusion injury, traumatic brain injury, spinal cord injury, peripheral neuropathy, multiple sclerosis, Lupus, allergy, cardiometabolic diseases, obesity, type II diabetes mellitus, type I diabetes mellitis, and NASH/cirrhosis.

The term “cytokine” as used herein refers to small soluble protein substances secreted by cells, which have a variety of effects on other cells. Cytokines mediate many important physiological functions including growth, development, wound healing, and the immune response. They act by binding to their cell-specific receptors located in the cell membrane that allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNF-α and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The terms “deletion” and “deletion mutation” are used interchangeably herein to refer to that in which a base or bases are lost from a DNA sequence.

The terms “disease” or “disorder” as used herein refer to an impairment of health or a condition of abnormal functioning.

The term “domain” as used herein refers to a region of a protein with a characteristic tertiary structure and function and to any of the three-dimensional subunits of a protein that together make up its tertiary structure formed by folding its linear peptide chain.

The term “therapeutic domain” (also referred to as “TD”) as used herein refers to a peptide, peptide segment, or variant or derivative thereof, with substantial identity to peptide KALARQLGVAA (SEQ ID NO: 2), or segment thereof. Therapeutic domains generally are not capable of penetrating the plasma membrane of mammalian cells and when contacted with a kinase enzyme, inhibit the kinase enzyme such that the kinase activity of the kinase enzyme is reduced. For example, according to one embodiment, a therapeutic domain according to the described invention may inhibit an MK2 kinase such that the activity of the MK2 kinase is about 99% of the activity of an uninhibited MK2 kinase. According to another embodiment, a therapeutic domain may inhibit an MK2 kinase such that the activity of the MK2 kinase is about 95% of that of an uninhibited MK2 kinase. According to another embodiment, a therapeutic domain may inhibit an MK2 kinase such that the activity of the MK2 kinase is about 90% of that of an uninhibited MK2 kinase. According to another embodiment, a therapeutic domain may inhibit an MK2 kinase such that the activity of the MK2 kinase is about 85% of that of an uninhibited MK2 kinase. According to another embodiment, a therapeutic domain may inhibit an MK2 kinase such that the activity of the MK2 kinase is about 80% of that of an uninhibited MK2 kinase. A therapeutic domain may inhibit an MK2 kinase such that the activity of the MK2 kinase is about 75% of that of an uninhibited MK2 kinase. According to another embodiment, a therapeutic domain may inhibit an MK2 kinase such that the activity of the MK2 kinase is about 70% of that of an uninhibited MK2 kinase. According to another embodiment, a therapeutic domain may inhibit an MK2 kinase such that the activity of MK2 kinase is about 65% of that of an uninhibited MK2 kinase. According to another embodiment, a therapeutic domain may inhibit an MK2 kinase such that the activity of the MK2 kinase is about 60% of that of an uninhibited MK2 kinase. According to another embodiment, a therapeutic domain may inhibit an MK2 kinase such that the activity of the MK2 kinase is about 55% of that of an uninhibited MK2 kinase. According to another embodiment, a therapeutic domain may inhibit an MK2 kinase such that the activity of the MK2 kinase is about 50% of that of an uninhibited MK2 kinase. According to another embodiment, a therapeutic domain may inhibit an MK2 kinase such that the activity of the MK2 kinase is about 45% of that of an uninhibited MK2 kinase. According to another embodiment, a therapeutic domain may inhibit an MK2 kinase such that the activity of the MK2 kinase is about 40% of that of an uninhibited MK2 kinase. According to another embodiment, a therapeutic domain may inhibit an MK2 kinase such that the activity of MK2 kinase is about 35% of that of an uninhibited MK2 kinase. According to another embodiment, a therapeutic domain may inhibit an MK2 kinase such that the activity of the MK2 kinase is about 30% of that of an uninhibited MK2 kinase. According to another embodiment, a therapeutic domain may inhibit an MK2 kinase such that the activity of the MK2 kinase is about 25% of that of an uninhibited MK2 kinase. According to another embodiment, a therapeutic domain may inhibit an MK2 kinase such that the activity of the MK2 kinase is about 20% of that of an uninhibited MK2 kinase. According to another embodiment, a therapeutic domain may inhibit an MK2 kinase such that the activity of the MK2 kinase is about 15% of that of an uninhibited MK2 kinase. According to another embodiment, a therapeutic domain may inhibit an MK2 kinase such that the activity of the MK2 kinase is about 10% of that of an uninhibited MK2 kinase. According to another embodiment, a therapeutic domain may inhibit an MK2 kinase such that the activity of the MK2 kinase is about 9% of that of an uninhibited MK2 kinase. According to another embodiment, a therapeutic domain may inhibit an MK2 kinase such that the activity of the MK2 kinase is about 8% of that of an uninhibited MK2 kinase. According to another embodiment, a therapeutic domain may inhibit an MK2 kinase such that the activity of the MK2 kinase is about 7% of that of an uninhibited MK2 kinase. According to another embodiment, a therapeutic domain may inhibit an MK2 kinase such that the activity of the MK2 kinase is about 6% of that of an uninhibited MK2 kinase. According to another embodiment, a therapeutic domain may inhibit an MK2 kinase such that the activity of the MK2 kinase is about 5% of that of an uninhibited MK2 kinase. According to another embodiment, a therapeutic domain may inhibit an MK2 kinase such that the activity of the MK2 kinase is about 4% of that of an uninhibited MK2 kinase. According to another embodiment, a therapeutic domain may inhibit an MK2 kinase such that the activity of the MK2 kinase is about 3% of that of an uninhibited MK2 kinase. According to another embodiment, a therapeutic domain may inhibit an MK2 kinase such that the activity of the MK2 kinase is about 2% of that of an uninhibited MK2 kinase. According to another embodiment, a therapeutic domain may inhibit an MK2 kinase such that the activity of the MK2 kinase is about 1% of that of an uninhibited MK2 kinase. According to another embodiment, a therapeutic domain may inhibit an MK2 kinase such that the activity of the MK2 kinase is about 0.1% of that of an uninhibited MK2 kinase. According to another embodiment, a therapeutic domain may inhibit an MK2 kinase such that the activity of the MK2 kinase is about 0.01% of that of an uninhibited MK2 kinase.

The term “protein transduction domain” (also referred to as “PTD”, “Trojan peptide”, “membrane translocating sequence”, “cell permeable protein”, “CPP”) as used herein refers to a class of peptides generally capable of penetrating the plasma membrane of mammalian cells. The term “protein transduction domain” as used herein also refers to a peptide, peptide segment, or variant or derivative thereof, with substantial identity to peptide YARAAARQARA (SEQ ID NO: 26), or a functional segment thereof. The term “protein transduction domain” as used herein also refers to a peptide, peptide segment, or variant or derivative thereof, which is functionally equivalent to SEQ ID NO: 26. PTDs generally are 10-16 amino acids in length. PTDs are capable of transporting compounds of many types and molecular weights across mammalian cells. Such compounds include, but are not limited to, effector molecules, such as proteins, DNA, conjugated peptides, oligonucleotides, and small particles such as liposomes. PTDs chemically linked or fused to other proteins (“fusion proteins”) still are able to penetrate the plasma membrane and enter cells.

The term “extracellular matrix” as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fribronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans.

The term “functional equivalent” or “functionally equivalent” are used interchangeably herein to refer to substances, molecules, polynucleotides, proteins, peptides, or polypeptides having similar or identical effects or use. A polypeptide functionally equivalent to polypeptide YARAAARQARAKALARQLGVAA (SEQ ID NO: 1), for example, may have a biologic activity, e.g., an inhibitory activity, kinetic parameters, salt inhibition, a cofactor-dependent activity, and/or a functional unit size that is substantially similar or identical to the expressed polypeptide of SEQ ID NO: 1.

Examples of polypeptides functionally equivalent to YARAAARQARAKALARQLGVAA (SEQ ID NO: 1) include, but are not limited to, a polypeptide of amino acid sequence WLRRIKAWLRRIKALNRQLGVAA (SEQ ID NO: 3), a polypeptide of amino acid sequence FAKLAARLYRKALARQLGVAA (SEQ ID NO: 4), a polypeptide of amino acid sequence KAFAKLAARLYRKALARQLGVAA (SEQ ID NO: 5), a polypeptide of amino acid sequence YARAAARQARAKALNRQLGVAA (SEQ ID NO: 6), a polypeptide of amino acid sequence YARAAARQARAKALARQLAVA (SEQ ID NO: 7), a polypeptide of amino acid sequence YARAAARQARAKALARQLGVA (SEQ ID NO: 8), a polypeptide of amino acid sequence YARAAARQARAKALNRQLAVA (SEQ ID NO: 9), a polypeptide of amino acid sequence YARAAARQARAKALNRQLGVA (SEQ ID NO: 10), a polypeptide of amino acid sequence YARAAARQARAKALNRQLGVAA (SEQ ID NO: 11), a polypeptide of amino acid sequence YARAAARQARAKALNRQLAVAA (SEQ ID NO: 12).

The term “MK2i peptide” or “MK2i” or “MMI-0100” as used interchangeably herein refers to a peptide of amino acid sequence YARAAARQARAKALARQLGVAA (SEQ ID NO: 1) comprising a fusion protein in which a protein transduction domain (PTD; YARAAARQARA; SEQ ID NO: 26) is operatively linked to a therapeutic domain (KALARQLGVAA; SEQ ID NO: 2).

Examples of polypeptides functionally equivalent to the therapeutic domain (TD; KALARQLGVAA; SEQ ID NO: 2) of the polypeptide YARAAARQARAKALARQLGVAA (SEQ ID NO: 1) include, but are not limited to, a polypeptide of amino acid sequence KALARQLAVA (SEQ ID NO: 13), a polypeptide of amino acid sequence KALARQLGVA (SEQ ID NO: 14), a polypeptide of amino acid sequence KALARQLGVAA (SEQ ID NO: 15), a polypeptide of amino acid sequence KALNRQLGVAA (SEQ ID NO: 16), a polypeptide of amino acid sequence KAANRQLGVAA (SEQ ID NO: 17), a polypeptide of amino acid sequence KALNAQLGVAA (SEQ ID NO: 18), a polypeptide of amino acid sequence KALNRALGVAA (SEQ ID NO: 19), a polypeptide of amino acid sequence KALNRQAGVAA (SEQ ID NO: 20), a polypeptide of amino acid sequence KALNRQLAVA (SEQ ID NO: 21), a polypeptide of amino acid sequence KALNRQLAVAA (SEQ ID NO: 22), a polypeptide of amino acid sequence KALNRQLGAAA (SEQ ID NO: 23), a polypeptide of amino acid sequence KALNRQLGVA (SEQ ID NO: 24), a polypeptide of amino acid sequence KKKALNRQLGVAA (SEQ ID NO: 25).

Examples of polypeptides functionally equivalent to the protein transduction domain (PTD; YARAAARQARA; SEQ ID NO: 26) of the polypeptide YARAAARQARAKALARQLGVAA (SEQ ID NO: 1) include, but are not limited to, a polypeptide of amino acid sequence WLRRIKAWLRRIKA (SEQ ID NO: 27), a polypeptide of amino acid sequence WLRRIKA (SEQ ID NO: 28), a polypeptide of amino acid sequence YGRKKRRQRRR (SEQ ID NO: 29), a polypeptide of amino acid sequence WLRRIKAWLRRI (SEQ ID NO: 30), a polypeptide of amino acid sequence FAKLAARLYR (SEQ ID NO: 31), a polypeptide of amino acid sequence KAFAKLAARLYR (SEQ ID NO: 32).

The term “fusion protein” as used herein refers to a protein or polypeptide constructed by combining multiple protein domains or polypeptides for the purpose of creating a single polypeptide or protein with functional properties derived from each of the original proteins or polypeptides. Creation of a fusion protein may be accomplished by operatively ligating or linking two different nucleotides sequences that encode each protein domain or polypeptide via recombinant DNA technology, thereby creating a new polynucleotide sequences that codes for the desired fusion protein. Alternatively, a fusion protein maybe created by chemically joining the desired protein domains.

The term “graft” as used herein refers to a tissue or organ transplanted from a donor to a recipient. It includes, but is not limited to, a self tissue transferred from one body site to another in the same individual (“autologous graft”), a tissue transferred between genetically identical individuals or sufficiently immunologically compatible to allow tissue transplant (“syngeneic graft”), a tissue transferred between genetically different members of the same species (“allogeneic graft” or “allograft”), and a tissue transferred between different species (“xenograft”).

The term “synthetic graft” or “prosthetic graft” as used herein refers to a graft made with artificial materials or natural polymers. Examples of such artificial materials include, but are not limited to, polytetrafluoroethylene (PTFE), polyester (Dacron®), polyester polylactic acid (PLA), polyglycolic acid (PGA), and polycaprolactone (PCL), polyurethanes, or combinations of the above materials. Examples of such natural polymers include, but are not limited to, heparin and collagen.

The term “tissue engineered graft” as used herein refers to a substitute tissue or organ made of cells, a scaffold, and signaling systems. Tissue engineered grafts are constructed by implanting or “seeding” cells into an artificial structure capable of supporting a three-dimensional tissue formation (“scaffold”). For example, a vascular tissue engineered graft may be made by implanting stem cells, progenitor cells, cells isolated from the saphenous vein wall, cells isolated from bone marrow, or from any other appropriate cell source and by culturing or expanding them in or on a biodegradable scaffold.

Bone marrow consists of a variety of precursor and mature cell types, including hematopoietic cells (the precursors of mature blood cells) and stromal cells (the precursors of a broad spectrum of connective tissue cells), both of which appear to be capable of differentiating into other cell types. The mononuclear fraction of bone marrow contains stromal cells, hematopoietic precursors, and endothelial precursors. CD34 is a hematopoietic stem cell antigen selectively expressed on hematopoietic stem and progenitor cells derived from human bone marrow, blood and fetal liver. Cells that express CD34 are termed CD34+. Stromal cells do not express CD34 and are therefore termed CD34−. CD34+ cells isolated from human blood may be capable of differentiating into cardiomyocytes, endothelial cells (the thin layer of cells that line, e.g., the interior surface of blood vessels), and smooth muscle cells in vivo (See Yeh et al., Circulation, 2003, 108: 2070-73) CD34+ cells represent approximately 1% of bone marrow derived nucleated cells; CD34 antigen also is expressed by immature endothelial cell precursors (mature endothelial cells do not express CD34) (Peichev, M. et al., Blood, 2000, 95: 952-58). In vitro, CD34+ cells derived from adult bone marrow give rise to a majority of the granulocyte/macrophage progenitor cells (CFU-GM), some colony-forming units-mixed (CFU-Mix) and a minor population of primitive erythroid progenitor cells (burst forming units, erythrocytes or BFU-E) (Yeh et al., Circulation, 2003, 108: 2070-73).

A three-dimensional scaffold is believed to be critical to replicate the in vivo milieu and to allow the cells to influence their own microenvironment. Scaffolds may serve to promote cell attachment and migration, to deliver and retain cells and biochemical factors, to enable diffusion of vital cell nutrients and expressed products, and to exert certain mechanical and biological influences to modify the behavior of the cell phase. A scaffold utilized for tissue reconstruction has several requisites. Such a scaffold should have a high porosity and an adequate pore size to facilitate cell seeding and diffusion of both cells and nutrients throughout the whole structure. Biodegradability of the scaffold is also an essential requisite. The scaffold should be absorbed by the surrounding tissues without the necessity of a surgical removal, such that the rate at which degradation occurs coincides as closely as possible with the rate of tissue formation. As cells are fabricating their own natural matrix structure around themselves, the scaffold provides structural integrity within the body and eventually degrades leaving the neotissue (newly formed tissue) to assume the mechanical load.

Several different materials (natural and synthetic, biodegradable and permanent) have been examined for use with scaffolds. Some biomaterials have been engineered to incorporate additional features such as injectability, synthetic manufacture, biocompatibility, non-immunogenicity, transparency, nanoscale fibers, low concentration, and resorption rates. For example, scaffolds may be constructed from synthetic materials, such as polylactic acid (PLA). PLA is a polyester which degrades within the human body to form a lactic acid byproduct which then is easily eliminated. Similar materials include polyglycolic acid (PGA) and polycaprolactone (PCL); PGA exhibits a faster degradation rate to lactic acid than PLA, while PCL exhibits a slower degradation rate. Scaffolds also may be constructed from natural materials. For example, several components of the extracellular matrix have been studied to evaluate their ability to support cell growth. Protein-based materials, such as collagen or fibrin, and polysaccharidic materials, such as chitosan or glycosaminoglycans (GAGs), have proved suitable in terms of cell compatibility.

The term “hemodialysis access graft” or “arteriovenous graft” as used herein refers to a vascular access that connects an artery to a vein using a graft implanted under the skin. The graft becomes an artificial vein that can be used repeatedly, for example, for needle placement and blood access during hemodialysis. Arteriovenous grafts can develop low blood flow, an indication of clotting or narrowing of the access. In this situation, the graft may require angioplasty to widen the small segment that is narrowed. Alternatively, surgery may be performed on the graft to replace the narrow segment.

As used herein the term “inflammation” refers to a physiologic response to infection and injury in which cells involved in detoxification and repair are mobilized to the compromised site by inflammatory mediators. The term “acute inflammation” as used herein, refers to inflammation, usually of sudden onset, characterized by the classical signs, with predominance of the vascular and exudative processes. The term “chronic inflammation” as used herein refers to inflammation of slow progress and marked chiefly by the formation of new connective tissue; it may be a continuation of an acute form or a prolonged low-grade form, and usually causes permanent tissue damage.

The term “inflammatory mediators” or “inflammatory cytokines” as used herein refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12).

Among the pro-inflammatory mediators, IL-1, IL-6, and TNF-α are known to activate hepatocytes in an acute phase response to synthesize acute-phase proteins that activate complement. Complement is a system of plasma proteins that interact with pathogens to mark them for destruction by phagocytes. Complement proteins can be activated directly by pathogens or indirectly by pathogen-bound antibody, leading to a cascade of reactions that occurs on the surface of pathogens and generates active components with various effector functions. IL-1, IL-6, and TNF-α also activate bone marrow endothelium to mobilize neutrophils, and function as endogenous pyrogens, raising body temperature, which helps eliminating infections from the body. A major effect of the cytokines is to act on the hypothalamus, altering the body's temperature regulation, and on muscle and fat cells, stimulating the catabolism of the muscle and fat cells to elevate body temperature. At elevated temperatures, bacterial and viral replication are decreased, while the adaptive immune system operates more efficiently.

The term “tumor necrosis factor” as used herein refers to a cytokine made by white blood cells in response to an antigen or infection, which induce necrosis (death) of tumor cells and possesses a wide range of pro-inflammatory actions. Tumor necrosis factor also is a multifunctional cytokine with effects on lipid metabolism, coagulation, insulin resistance, and the function of endothelial cells lining blood vessels.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include, interleukin-1 (IL-1), interleukin-10 (IL-1β), interleukin-6 (IL-6), interleukin-8 (IL-8), and interleukin-12 (IL-12).

The term “inhibit” and its various grammatical forms, including, but not limited to, “inhibiting” or “inhibition”, are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a second molecule that binds to a first molecule thereby decreasing the first molecule's activity. Enzyme inhibitors are molecules that bind to enzymes thereby decreasing enzyme activity. The binding of an inhibitor may stop a substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “injury” as used herein refers to damage or harm to a structure or function of the body caused by an outside agent or force, which may be physical or chemical.

The term “intimal hyperplasia” as used herein refers to a thickening of the tunica intima (the innermost layer of an artery or vein) of a vessel as a complication of a reconstruction procedure or endarterectomy (a surgical stripping of a fat-encrusted, thickened arterial lining so as to open or widen the artery for improved blood circulation). Intimal hyperplasia is the universal response of a vessel to injury, and it involves a coordinated stimulation of smooth muscle cells by mechanical, cellular, and humoral factors, which leads to proliferation, migration and extracellular matrix deposition. For example, studies have reported that the increased level of IL-6, as regulated by IL-1β and TNF-α, promotes the formation of atheromatous plaques (accumulation of macrophages or lipids, calcium, and a variable amount of fibrous connective tissue). (Kornman, K. et al. J. Perio. Res. 34(7):353-357. 2006; Libby, P., et al. Circulation. 86(6 Suppl): III47-52. 1992)), each incorporated herein by reference in its entirety.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95% free of, or more than about 99% free of. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state. For example, a naturally occurring nucleic acid becomes an isolated nucleic acid if it is altered, or if it is transcribed from DNA that has been altered, by means of human intervention performed within the cell from which it originates. (See, for example, Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868, each incorporated herein by reference in its entirety). Likewise, a naturally occurring nucleic acid (for example, a promoter) becomes isolated if it is introduced by non-naturally occurring means to a locus of the genome not native to that nucleic acid. Nucleic acids that are “isolated” as defined herein also are referred to as “heterologous” nucleic acids.

The term “kinase” as used herein refers to a type of enzyme that transfers phosphate groups from high-energy donor molecules to specific target molecules or substrates. High-energy donor groups may include, but are not limited, to ATP.

The term “MK2 kinase” or “MK2” as used herein refers to mitogen-activated protein kinase-activated protein kinase 2 (also referred to as “MAPKAPK2”, “MAPKAP-K2”, “MK2”), which is a member of the serine/threonine (Ser/Thr) protein kinase family.

The term “nucleic acid” is used herein to refer to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” is used herein to refer to a chemical compound that consists of a heterocyclic base, a sugar, and one or more phosphate groups. In the most common nucleotides, the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. Purines include adenine (A), and guanine (G); pyrimidines include cytosine (C), thymine (T), and uracil (U).

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity.”

(a) The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

(b) The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination.

(c) The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e) The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid.

The phrase “operatively linked” as used herein refers to a peoptide bond through which two or more protein domains or polypeptides are ligated or combined via recombinant DNA technology or chemical reaction such that each protein domain or polypeptide of the resulting fusion protein retains its original function. For example, SEQ ID NO: 1 is constructed by operatively linking a protein transduction domain (SEQ ID NO: 26) with a therapeutic domain (SEQ ID NO: 2), thereby creating a fusion protein that possesses both the cell penetrating function of SEQ ID NO: 26 and the MK2 kinase inhibitor function of SEQ ID NO: 2.

The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

The term “patency” as used herein refers to the state or quality of being open, expanded, or unblocked. For example, vascular patency refers to the condition of blood vessels not being blocked or obstructed.

As used herein the term “pharmaceutically acceptable carrier” refers to any substantially non-toxic carrier conventionally useable for administration of pharmaceuticals in which the isolated polypeptide of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

The term “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein that protein is specifically reactive to antibodies elicited to the same protein but consisting entirely of naturally occurring amino acids.

The terms “polypeptide” and “protein” also are used herein in their broadest sense to refer to a sequence of subunit amino acids, amino acid analogs, or peptidomimetics. The subunits are linked by peptide bonds, except where noted. The polypeptides described herein may be chemically synthesized or recombinantly expressed. Polypeptides of the described invention also can be synthesized chemically. Synthetic polypeptides, prepared using the well known techniques of solid phase, liquid phase, or peptide condensation techniques, or any combination thereof, can include natural and unnatural amino acids. Amino acids used for peptide synthesis may be standard Boc (N-α-amino protected N-α-t-butyloxycarbonyl) amino acid resin with the standard deprotecting, neutralization, coupling and wash protocols of the original solid phase procedure of Merrifield (J. Am. Chem. Soc., 1963, 85:2149-2154), or the base-labile N-α-amino protected 9-fluorenylmethoxycarbonyl (Fmoc) amino acids first described by Carpino and Han (J. Org. Chem., 1972, 37:3403-3409). Both Fmoc and Boc N-α-amino protected amino acids can be obtained from Sigma, Cambridge Research Biochemical, or other chemical companies familiar to those skilled in the art. In addition, the polypeptides can be synthesized with other N-α-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, Int. J. Pept. Protein Res., 1990, 35:161-214, or using automated synthesizers. The polypeptides of the invention may comprise D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Synthetic amino acids include ornithine for lysine, and norleucine for leucine or isoleucine. In addition, the polypeptides can have peptidomimetic bonds, such as ester bonds, to prepare peptides with novel properties. For example, a peptide may be generated that incorporates a reduced peptide bond, i.e., R1-CH₂—NH—R2, where R1 and R2 are amino acid residues or sequences. A reduced peptide bond may be introduced as a dipeptide subunit. Such a polypeptide would be resistant to protease activity, and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids.

The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. In some embodiments, the peptide is of any length or size.

The term “progenitor cell” as used herein refers to an immature cell in the bone marrow that may be isolated by growing suspensions of marrow cells in culture dishes with added growth factors. Progenitor cells mature into precursor cells that mature into blood cells. Progenitor cells are referred to as colony-forming units (CFU) or colony-forming cells (CFC). The specific lineage of a progenitor cell is indicated by a suffix, such as, but not limited to, CFU-E (erythrocytic), CFU-GM (granulocytic/macrophage), and CFU-GEMM (pluripotent hematopoietic progenitor).

The term “similar” is used interchangeably with the terms analogous, comparable, or resembling, meaning having traits or characteristics in common.

The term “solution” as used herein refers to a homogeneous mixture of two or more substances. It is frequently, though not necessarily, a liquid. In a solution, the molecules of the solute (or dissolved substance) are uniformly distributed among those of the solvent.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent.

The term “stem cells” refers to undifferentiated cells having high proliferative potential with the ability to self-renew that can generate daughter cells that can undergo terminal differentiation into more than one distinct cell phenotype.

The term “stenosis” as used herein refers to an abnormal narrowing, stricture or obstruction of a passage or tubular structure.

The term “suspension” as used herein refers to a dispersion (mixture) in which a finely-divided species is combined with another species, with the former being so finely divided and mixed that it doesn't rapidly settle out. In everyday life, the most common suspensions are those of solids in liquid.

The terms “subject” or “individual” or “patient” are used interchangeably to refer to a member of an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human.

The phrase “subject in need of such treatment” as used herein refers to a patient who (i) will receive vascular graft; (ii) is receiving vascular graft; or (iii) has received vascular graft. In some other embodiments, the phrase “subject in need of such treatment” also is used to refer to a patient who (i) will suffer from a vascular disease comprising intimal hyperplasia; (ii) is suffering from a vascular disease comprising intimal hyperplasia; or (iii) has suffered from a vascular disease comprising intimal hyperplasia. In some other embodiments, the phrase “subject in need of such treatment” also is used to refer to a patient who (i) will be administered at least one polypeptide of the invention; (ii) is receiving at least one polypeptide of the invention; or (iii) has received at least one polypeptide of the invention, unless the context and usage of the phrase indicates otherwise.

The term “substitution” is used herein to refer to a situation in which a base or bases are exchanged for another base or bases in a DNA sequence. Substitutions may be synonymous substitutions or nonsynonymous substitutions. As used herein, “synonymous substitutions” refer to substitutions of one base for another in an exon of a gene coding for a protein, such that the amino acid sequence produced is not modified. The term “nonsynonymous substitutions” as used herein refer to substitutions of one base for another in an exon of a gene coding for a protein, such that the amino acid sequence produced is modified.

The terms “therapeutically effective amount”, an “amount effective”, or “pharmaceutically effective amount” of an active agent are used interchangeably to refer to an amount that is sufficient to provide the intended benefit of treatment. An effective amount of an active agent that can be employed according to the described invention generally ranges from generally about 0.01 mg/kg body weight to about 100 g/kg body weight. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods.

The term “topical” as used herein refers to administration of an inventive composition at, or immediately beneath, the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably.

Topical administration also may involve the use of transdermal administration such as transdermal patches or iontophoresis devices which are prepared according to techniques and procedures well known in the art. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation. Patches suitable for use in the described invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated by reference in its entirety. These patches are well known in the art and generally available commercially.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The terms “variants”, “mutants”, and “derivatives” are used herein to refer to nucleotide or polypeptide sequences with substantial identity to a reference nucleotide or polypeptide sequence. The differences in the sequences may be the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce polypeptide variants of polypeptide YARAAARQARAKALARQLGVAA (SEQ ID NO: 1) having single or multiple amino acid substitutions, deletions, additions or replacements, but functionally equivalent to SEQ ID NO: 1. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (e) variants in which a target protein is fused with another peptide or polypeptide such as a fusion partner, a protein tag or other chemical moiety, that may confer useful properties to the target protein, for example, an epitope for an antibody. The techniques for obtaining such variants, including, but not limited to, genetic (suppressions, deletions, mutations, etc.), chemical, and enzymatic techniques, are known to the skilled artisan. As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “vascular access” as used herein refer to the site where blood is removed and returned during dialysis. It is desirable that a vascular access allow continuous high volumes of blood flow to maximize the amount of blood cleansed during hemodialysis treatments.

The term “vascular disease” as used herein refers to a condition that affects blood vessels (e.g., arteries, veins, and capillaries) carrying blood throughout the body. According to some embodiments, a condition that affect blood vessels includes restriction in diameter of a blood vessel and changes in vascular permeability. Vascular disease usually is caused by atherosclerosis, a hardening of the walls of a blood vessel by a build-up of lipid deposits (plaque) on the inner lining of the vessel. Atherosclerosis narrows a blood vessel and causes less blood to flow to the receiving tissue, leading to ischemic injury (meaning injury due to a decrease in blood supply and oxygen to the cells of the receiving tissue). For example, coronary artery disease is the most common form of arterial vascular disease that leads to ischemic injury to the heart. Similarly, peripheral arteries, located outside the heart and brain, may develop atherosclerosis in the arteries supplying blood to the kidneys, stomach, arms, legs, and feet.

The term “vascular spasm” or “vasospasm” as used herein refers to an involuntary contraction of vascular smooth muscle cells that line blood vessels that can acutely reduce blood supply and tissue oxygenation, which may lead to vasoconstriction, tissue ischemia and death.

The term “vehicle” as used herein refers to a substance that facilitates the use of a drug or other material that is mixed with it.

Compositions: Therapeutic Peptides that Inhibits MK2 Kinase

According to one aspect, the described invention provides an MK2 kinase inhibiting pharmaceutical composition for treating a vascular graft failure or a vascular disease comprising intimal hyperplasia, wherein the pharmaceutical composition comprises a therapeutically effective amount of a polypeptide of the amino acid sequence YARAAARQARAKALARQLGVAA (SEQ ID NO: 1) or a functional equivalent thereof, and a pharmaceutically acceptable carrier thereof.

According to some embodiments, the functional equivalent of the polypeptide YARAAARQARAKALARQLGVAA (SEQ ID NO: 1) has a substantial sequence identity to amino acid sequence YARAAARQARAKALARQLGVAA (SEQ ID NO: 1). According to another embodiments, the functional equivalent of the polypeptide YARAAARQARAKALARQLGVAA (SEQ ID NO: 1) has at least 70 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 80 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 90 percent sequence identity to amino acid sequence YARAAARQARAKALARQLGVAA (SEQ ID NO: 1). According to another embodiment, the functional equivalent of the polypeptide YARAAARQARAKALARQLGVAA (SEQ ID NO: 1) has at least 95 percent sequence identity to amino acid sequence YARAAARQARAKALARQLGVAA (SEQ ID NO: 1).

According to another embodiment, the functional equivalent of the polypeptide YARAAARQARAKALARQLGVAA (SEQ ID NO: 1) is of amino acid sequence WLRRIKAWLRRIKALNRQLGVAA (SEQ ID NO: 3). According to another embodiment, the functional equivalent of the polypeptide YARAAARQARAKALARQLGVAA (SEQ ID NO: 1) is of amino acid sequence FAKLAARLYRKALARQLGVAA (SEQ ID NO: 4). According to another embodiment, the functional equivalent of the polypeptide YARAAARQARAKALARQLGVAA (SEQ ID NO: 1) is of amino acid sequence KAFAKLAARLYRKALARQLGVAA (SEQ ID NO: 5). According to another embodiment, the functional equivalent of the polypeptide YARAAARQARAKALARQLGVAA (SEQ ID NO: 1) is of amino acid sequence YARAAARQARAKALNRQLGVAA (SEQ ID NO: 6). According to another embodiment, the functional equivalent of the polypeptide YARAAARQARAKALARQLGVAA (SEQ ID NO: 1) is of amino acid sequence YARAAARQARAKALARQLAVA (SEQ ID NO: 7). According to another embodiment, the functional equivalent of the polypeptide YARAAARQARAKALARQLGVAA (SEQ ID NO: 1) is of amino acid sequence YARAAARQARAKALARQLGVA (SEQ ID NO: 8). According to another embodiment, the functional equivalent of the polypeptide YARAAARQARAKALARQLGVAA (SEQ ID NO: 1) is of amino acid sequence YARAAARQARAKALNRQLAVA (SEQ ID NO: 9). According to another embodiment, the functional equivalent of the polypeptide YARAAARQARAKALARQLGVAA (SEQ ID NO: 1) is of amino acid sequence YARAAARQARAKALNRQLGVA (SEQ ID NO: 10). According to another embodiment, the functional equivalent of the polypeptide YARAAARQARAKALARQLGVAA (SEQ ID NO: 1) is of amino acid sequence YARAAARQARAKALNRQLGVAA (SEQ ID NO: 11). According to another embodiment, the functional equivalent of the polypeptide YARAAARQARAKALARQLGVAA (SEQ ID NO: 1) is of amino acid sequence YARAAARQARAKALNRQLAVAA (SEQ ID NO: 12)

According to some other embodiments, the functional equivalent of the polypeptide YARAAARQARAKALARQLGVAA (SEQ ID NO: 1) is a fusion protein comprising a first polypeptide operatively linked to a second polypeptide, wherein the first polypeptide is of amino acid sequence YARAAARQARA (SEQ ID NO: 26), and the second polypeptide comprises a therapeutic domain whose sequence has a substantial identity to amino acid sequence KALARQLGVAA (SEQ ID NO: 2). According to a further embodiment, the second polypeptide has at least 70 percent sequence identity to amino acid sequence KALARQLGVAA (SEQ ID NO: 2). According to some other embodiments, the second polypeptide has at least 80 percent sequence identity to amino acid sequence KALARQLGVAA (SEQ ID NO: 2). According to some other embodiments, the second polypeptide has at least 90 percent sequence identity to amino acid sequence KALARQLGVAA (SEQ ID NO: 2). According to some other embodiments, the second polypeptide has at least 95 percent sequence identity to amino acid sequence KALARQLGVAA (SEQ ID NO: 2).

According to another embodiment, the second polypeptide is a polypeptide of amino acid sequence KALARQLAVA (SEQ ID NO: 13). According to another embodiment, the second polypeptide is a polypeptide of amino acid sequence KALARQLGVA (SEQ ID NO: 14). According to another embodiment, the second polypeptide is a polypeptide of amino acid sequence KALARQLGVAA (SEQ ID NO: 15). According to another embodiment, the second polypeptide is a polypeptide of amino acid sequence KALNRQLGVAA (SEQ ID NO: 16). According to another embodiment, the second polypeptide is a polypeptide of amino acid sequence KAANRQLGVAA (SEQ ID NO: 17). According to another embodiment, the second polypeptide is a polypeptide of amino acid sequence KALNAQLGVAA (SEQ ID NO: 18). According to another embodiment, the second polypeptide is a polypeptide of amino acid sequence KALNRALGVAA (SEQ ID NO: 19). According to another embodiment, the second polypeptide is a polypeptide of amino acid sequence of KALNRQAGVAA (SEQ ID NO: 20). According to another embodiment, the second polypeptide is a polypeptide of amino acid sequence KALNRQLAVA (SEQ ID NO: 21). According to another embodiment, the second polypeptide is a polypeptide of amino acid sequence KALNRQLAVAA (SEQ ID NO: 22). According to another embodiment, the second polypeptide is a polypeptide of amino acid sequence KALNRQLGAAA (SEQ ID NO: 23). According to another embodiment, the second polypeptide is a polypeptide of amino acid sequence of KALNRQLGVA (SEQ ID NO: 24). According to another embodiment, the second polypeptide is a polypeptide of amino acid sequence KKKALNRQLGVAA (SEQ ID NO: 25).

According to some other embodiments, the functional equivalent of the polypeptide YARAAARQARAKALARQLGVAA (SEQ ID NO: 1) is a fusion protein comprising a first polypeptide operatively linked to a second polypeptide, wherein the first polypeptide comprises a protein transduction domain functionally equivalent to YARAAARQARA (SEQ ID NO: 26), and the second polypeptide is of amino acid sequence KALARQLGVAA (SEQ ID NO: 2). According to a further embodiment, the first polypeptide is a polypeptide of amino acid sequence WLRRIKAWLRRIKA (SEQ ID NO: 27). According to another embodiment, the first polypeptide is a polypeptide of amino acid sequence WLRRIKA (SEQ ID NO: 28). According to another embodiment, the first polypeptide is a polypeptide of amino acid sequence YGRKKRRQRRR (SEQ ID NO: 29). According to another embodiment, the first polypeptide is a polypeptide of amino acid sequence WLRRIKAWLRRI (SEQ ID NO: 30). According to another embodiment, the first polypeptide is a polypeptide of amino acid sequence FAKLAARLYR (SEQ ID NO: 31). According to another embodiment, the first polypeptide is a polypeptide of amino acid sequence KAFAKLAARLYR (SEQ ID NO: 32).

According to another aspect, the described invention also provides an isolated nucleic acid that encodes a protein sequence with at least 70% amino acid sequence identity to amino acid sequence YARAAARQARAKALARQLGVAA (SEQ ID NO: 1). According to some such embodiments, the isolated nucleic acid encodes a protein sequence with at least 80% amino acid sequence identity to amino acid sequence YARAAARQARAKALARQLGVAA (SEQ ID NO: 1). According to some such embodiments, the isolated nucleic acid encodes a protein sequence with at least 90% amino acid sequence identity to amino acid sequence YARAAARQARAKALARQLGVAA (SEQ ID NO: 1). According to some such embodiments, the isolated nucleic acid encodes a protein sequence with at least 95% amino acid sequence identity to amino acid sequence YARAAARQARAKALARQLGVAA (SEQ ID NO: 1).

According to one embodiment of the invention, the therapeutically effective amount of the therapeutic inhibitor peptide of the pharmaceutical composition is of an amount from about 0.000001 mg/kg body weight to about 100 mg/kg body weight. According to another embodiment, the therapeutically effective amount of the therapeutic inhibitory peptide of the pharmaceutical composition is of an amount from about 0.00001 mg/kg body weight to about 100 mg/kg body weight. According to another embodiment, the therapeutically effective amount of the therapeutic inhibitory peptide of the pharmaceutical composition is of an amount from about 0.0001 mg/kg body weight to about 100 mg/kg body weight. According to another embodiment, the therapeutically effective amount of the therapeutic inhibitory peptide of the pharmaceutical composition is of an amount from about 0.001 mg/kg body weight to about 100 mg/kg body weight. According to another embodiment, the therapeutically effective amount of the therapeutic inhibitory peptide of the pharmaceutical composition is of an amount from about 0.01 mg/kg body weight to about 100 mg/kg body weight. According to another embodiment, the therapeutically effective amount of the therapeutic inhibitory peptide of the pharmaceutical composition is of an amount from about 0.1 mg/kg body weight to about 100 mg/kg body weight. According to another embodiment, the therapeutically effective amount of the therapeutic inhibitory peptide of the pharmaceutical composition is of an amount from about 1 mg/kg body weight to about 100 mg/kg body weight. According to another embodiment, the therapeutically effective amount of the therapeutic inhibitory peptide of the pharmaceutical composition is of an amount from about 10 mg/kg body weight to about 100 mg/kg body weight. According to another embodiment, the therapeutically effective amount of the therapeutic inhibitory peptide of the pharmaceutical composition is of an amount from about 20 mg/kg body weight to about 100 mg/kg body weight. According to another embodiment, the therapeutically effective amount of the therapeutic inhibitory peptide of the pharmaceutical composition is of an amount from about 30 mg/kg body weight to about 100 mg/kg body weight. According to another embodiment, the therapeutically effective amount of the therapeutic inhibitory peptide of the pharmaceutical composition is of an amount from about 40 mg/kg body weight to about 100 mg/kg body weight. According to another embodiment, the therapeutically effective amount of the therapeutic inhibitory peptide of the pharmaceutical composition is of an amount from about 50 mg/kg body weight to about 100 mg/kg body weight. According to another embodiment, the therapeutically effective amount of the therapeutic inhibitory peptide of the pharmaceutical composition is of an amount from about 60 mg/kg body weight to about 100 mg/kg body weight. According to another embodiment, the therapeutically effective amount of the therapeutic inhibitory peptide of the pharmaceutical composition is of an amount from about 70 mg/kg body weight to about 100 mg/kg body weight. According to another embodiment, the therapeutically effective amount of the therapeutic inhibitory peptide of the pharmaceutical composition is of an amount from about 80 mg/kg body weight to about 100 mg/kg body weight. According to another embodiment, the therapeutically effective amount of the therapeutic inhibitory peptide of the pharmaceutical composition is of an amount from about 90 mg/kg body weight to about 100 mg/kg body weight. According to another embodiment, the therapeutically effective amount of the therapeutic inhibitor peptide of the pharmaceutical composition is of an amount from about 0.000001 mg/kg body weight to about 90 mg/kg body weight. According to another embodiment, the therapeutically effective amount of the therapeutic inhibitor peptide of the pharmaceutical composition is of an amount from about 0.000001 mg/kg body weight to about 80 mg/kg body weight. According to another embodiment, the therapeutically effective amount of the therapeutic inhibitor peptide of the pharmaceutical composition is of an amount from about 0.000001 mg/kg body weight to about 70 mg/kg body weight. According to another embodiment, the therapeutically effective amount of the therapeutic inhibitor peptide of the pharmaceutical composition is of an amount from about 0.000001 mg/kg body weight to about 60 mg/kg body weight. According to another embodiment, the therapeutically effective amount of the therapeutic inhibitor peptide of the pharmaceutical composition is of an amount from about 0.000001 mg/kg body weight to about 50 mg/kg body weight. According to another embodiment, the therapeutically effective amount of the therapeutic inhibitor peptide of the pharmaceutical composition is of an amount from about 0.000001 mg/kg body weight to about 40 mg/kg body weight. According to another embodiment, the therapeutically effective amount of the therapeutic inhibitor peptide is of an amount from about 0.000001 mg/kg body weight to about 30 mg/kg body weight. According to another embodiment, the therapeutically effective amount of the therapeutic inhibitor peptide of the pharmaceutical composition is of an amount from about 0.000001 mg/kg body weight to about 20 mg/kg body weight. According to another embodiment, the therapeutically effective amount of the therapeutic inhibitor peptide of the pharmaceutical composition is of an amount from about 0.000001 mg/kg body weight to about 10 mg/kg body weight. According to another embodiment, the therapeutically effective amount of the therapeutic inhibitor peptide of the pharmaceutical composition is of an amount from about 0.000001 mg/kg body weight to about 1 mg/kg body weight. According to another embodiment, the therapeutically effective amount of the therapeutic inhibitor peptide of the pharmaceutical composition is of an amount from about 0.000001 mg/kg body weight to about 0.1 mg/kg body weight. According to another embodiment, the therapeutically effective amount of the therapeutic inhibitor peptide of the pharmaceutical composition is of an amount from about 0.000001 mg/kg body weight to about 0.1 mg/kg body weight. According to another embodiment, the therapeutically effective amount of the therapeutic inhibitor peptide of the pharmaceutical composition is of an amount from about 0.000001 mg/kg body weight to about 0.01 mg/kg body weight. According to another embodiment, the therapeutically effective amount of the therapeutic inhibitor peptide of the pharmaceutical composition is of an amount from about 0.000001 mg/kg body weight to about 0.001 mg/kg body weight. According to another embodiment, the therapeutically effective amount of the therapeutic inhibitor peptide of the pharmaceutical composition is of an amount from about 0.000001 mg/kg body weight to about 0.0001 mg/kg body weight. According to another embodiment, the therapeutically effective amount of the therapeutic inhibitor peptide of the pharmaceutical composition is of an amount from about 0.000001 mg/kg body weight to about 0.00001 mg/kg body weight.

According to some embodiments, the polypeptide of the described invention is chemically synthesized. Such a synthetic polypeptide, prepared using the well known techniques of solid phase, liquid phase, or peptide condensation techniques, or any combination thereof, may include natural and unnatural amino acids. Amino acids used for peptide synthesis may be standard Boc (N-α-amino protected N-α-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile N-α-amino protected 9-fluorenylmethoxycarbonyl (Fmoc) amino acids first described by Carpino and Han (1972, J. Org. Chem. 37:3403-3409). Both Fmoc and Boc N-α-amino protected amino acids can be obtained from Sigma, Cambridge Research Biochemical, or other chemical companies familiar to those skilled in the art. In addition, the polypeptide may be synthesized with other N-α-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers, each incorporated by reference herein in its entirety.

According to some embodiments, the polypeptide of the invention comprises D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Examples of synthetic amino acid substitutions include ornithine for lysine, and norleucine for leucine or isoleucine.

Methods for Treating Vascular Graft Failure

According to another aspect, the described invention provides a method for treating or preventing failure of a vascular graft in a subject in need of such treatment, the method comprising administering a therapeutically effective amount of a pharmaceutical composition comprising a polypeptide of SEQ ID NO: 1 or a functional equivalent thereof, and a pharmaceutically acceptable carrier.

According to some embodiments, the step of administering is by implanting a biomedical device, wherein the device is a vascular graft, and wherein the composition is disposed on or in the graft.

According to some embodiments, the step of administering occurs parenterally.

According to some embodiments, the step of administering occurs topically.

According to some embodiments, the vascular graft is an autologous graft.

According to some embodiments, the vascular graft is a syngeneic graft.

According to some embodiments, the vascular graft is an allogeneic graft.

According to some embodiments, the vascular graft is a xenograft.

According to some embodiments, the vascular graft is a synthetic graft.

According to some embodiments, the vascular graft is a prosthetic graft.

According to some embodiments, the vascular graft is a tissue engineered graft.

According to some embodiments, the vascular graft is a vascular access graft.

According to some embodiments, the vascular graft is an arteriovenous graft.

According to some embodiments, the vascular graft is a coronary artery bypass graft.

According to some embodiments, the step of administering occurs at one time as a single dose, wherein the one time is during vascular graft surgery.

According to some embodiments, the step of administering is performed as a plurality of doses over a period of time. According to some such embodiments, the period of time is a day, a week, a month, a month, a year, or multiples thereof.

According to some embodiments, the step of administering is performed daily for a period of at least one week.

According to some embodiments, the step of administering is performed weekly for a period of at least one month.

According to some embodiments, the step of administering is performed monthly for a period of at least two months.

According to another embodiment, the step of administering is performed repeatedly over a period of at least one year.

According to another embodiment, the step of administering is performed at least once monthly.

According to another embodiment, the step of administering is performed at least once weekly.

According to another embodiment, the step of administering is performed at least once daily.

According to some embodiments, the method reduces stenosis of the vascular graft. According to some embodiments, the method reduces vasospasm of at least one blood vessel related to the vascular graft. According to some embodiments, the method reduces intimal hyperplasia of at least one blood vessel related to the vascular graft.

Methods for Treating Vascular Diseases Comprising Intimal Hyperplasia

According to another aspect, the present invention also provides a method for treating a vascular disease comprising intimal hyperplasia in a subject in need of such treatment, the method comprising administering a therapeutically effective amount of a pharmaceutical composition comprising a polypeptide of amino acid sequence YARAAARQARAKALARQLGVAA (SEQ ID NO: 1) or a functional equivalent thereof, and a pharmaceutically acceptable carrier.

According to another embodiment, the vascular disease is a pre-atherosclerotic intimal hyperplasia.

According to one embodiment, the vascular disease is an atherosclerosis.

According to some embodiments, the step of administering is by implanting a biomedical device, wherein the pharmaceutical composition is disposed on or in the device. According to some embodiments, the step of administering occurs parenterally. According to some embodiments, the step of administering occurs topically.

According to some embodiments, the step of administering occurs at one time as a single dose, wherein the one time is during vascular graft surgery.

According to some embodiments, the step of administering is performed as a plurality of doses over a period of time. According to some such embodiments, the period of time is a day, a week, a month, a month, a year, or multiples thereof.

According to some embodiments, the step of administering is performed daily for a period of at least one week.

According to some embodiments, the step of administering is performed weekly for a period of at least one month.

According to some embodiments, the step of administering is performed monthly for a period of at least two months.

According to another embodiment, the step of administering is performed repeatedly over a period of at least one year.

According to another embodiment, the step of administering is performed at least once monthly.

According to another embodiment, the step of administering is performed at least once weekly.

According to another embodiment, the step of administering is performed at least once daily.

According to some embodiments, the polypeptide of the described invention is combined with one or more carriers appropriate for the indicated route of administration.

According to some embodiments, the polypeptide may be linked to other compounds to promote an increased half-life in vivo, such as polyethylene glycol or dextran. Such linkage can be covalent or non-covalent as is understood by those of skill in the art. According to some other embodiments, the polypeptide may be encapsulated in a micelle such as a micelle made of poly(ethyleneglycol)-block-poly(polypropylenglycol) or poly(ethyleneglycol)-block-polyactide. According to some other embodiments, the polypeptide may be encapsulated in degradable nano- or micro-particles composed of degradable polyesters including, but not limited to, polylactic acid, polyglycolide, and polycaprolactone.

According to another embodiment, the polypeptide may be prepared in a solid form (including granules, powders or suppositories) or in a liquid form (e.g., solutions, suspensions, or emulsions).

According to another embodiment, the compositions of the described invention may be in the form of a dispersible dry powder for delivery by inhalation or insufflation (either through the mouth or through the nose). Dry powder compositions may be prepared by processes known in the art, such as lyophilization and jet milling, as disclosed in International Patent Publication No. WO 91/16038 and as disclosed in U.S. Pat. No. 6,921,527, the disclosures of which are incorporated by reference. The composition of the described invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. No. 4,227,522; U.S. Pat. No. 4,192,309; and U.S. Pat. No. 4,105,027. Suitable containers also include those used in conjunction with Glaxo's Ventolin® Rotohaler brand powder inhaler or Fison's Spinhaler® brand powder inhaler. Another suitable unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference in their entireties.

According to another embodiment, the carrier of the composition of the described invention includes a release agent, such as sustained release or delayed release carrier. In such embodiments, the carrier can be any material capable of sustained or delayed release of the polypeptide to provide a more efficient administration, e.g., resulting in less frequent and/or decreased dosage of the polypeptide, improve ease of handling, and extend or delay effects on diseases, disorders, conditions, syndromes, and the like, being treated, prevented or promoted. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

In yet another embodiment, the polypeptide of the invention may be applied in a variety of solutions. To be suitable, a formulations is sterile, dissolves sufficient amounts of the polypeptides, and is not harmful for the proposed application. For example, the compositions of the described invention may be formulated as aqueous suspensions wherein the active ingredient(s) is (are) in admixture with excipients suitable for the manufacture of aqueous suspensions.

Such excipients are suspending agents, for example, sodium carboxymethylcellulose, methylcellulose, hydroxy-propylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

Compositions of the described invention also may be formulated as oily suspensions by suspending the active ingredient in a vegetable oil, for example arachis oil, olive oil, sesame oil or coconut oil, or in a mineral oil, such as liquid paraffin. The oily suspensions may contain a thickening agent, for example, beeswax, hard paraffin or cetyl alcohol.

Compositions of the described invention also may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients also may be present.

Compositions of the described invention also may be in the form of an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil. Thus, the compositions of the invention may be in the form of an oil-in-water emulsion. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.), all of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges also is encompassed within the invention, subject to any specifically excluded limit in the stated range. Where the stated range includes one or both of the limits, ranges excluding either both of those included limits also are included in the invention.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the present invention, the preferred methods and materials are now described. All publications mentioned herein are incorporated herein by reference to disclose and described the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### I. Materials and Methods

MMI-0100 (YARAAARQARAKALARQLGVAA; SEQ ID NO: 1) Reconstitution/Dilution

MMI-0100 (YARAAARQARAKALARQLGVAA; SEQ ID NO: 1) was synthesized using standard Fmoc chemistry as described below in Example 1. For reconstitution, 114 mg of MMI-0100 (SEQ ID NO: 1; MW=2283.67 g/mol; Moerae Matrix, Inc.) was dissolved in 5 ml of phosphate-buffered saline (PBS) to yield a 0.01M stock solution, which was divided into 500 μl aliquots and stored at −20° C. Serial dilutions of stock solution were made to achieve appropriate drug concentrations for each study.

Cell Culture

Primary human aortic endothelial cells (HAEC) and primary human aortic smooth muscle cells (HASMC) were obtained from Invitrogen; primary human coronary artery endothelial cells (HCAEC) were obtained from Lonza. Appropriate growth media for these cell types were obtained from the same suppliers; formulations are summarized in Table 1, below.

TABLE 1
Cell culture media and supplement formulations.

| Cell type | Medium and supplements |
|---|---|
| HAEC | Medium 200 supplemented with LSGS (Low |
| (Gibco, Invitrogen) | Serum Growth Supplement), containing FBS (2% |
| | v/v), hydrocortisone (HC, 1 μg/mL), human |
| | epidermal growth factor (EGF, 10 ng/mL), Basic |
| | Fibroblast Growth Factor (bFGF, 3 ng/mL), |
| | gentamycin/amphotericin (GA) and heparin (10 |
| | μg/mL). |
| HCAEC | EGM Bullet Kit-EBM-2 Endothelial Basal |
| (Clonetics, Lonza) | Medium 2 supplemented with hEGF (10 ng/ml), |
| | Hydrocortisone (1.0 μg/ml), GA (50 μg/ml), FBS |
| | (5%), VEGF, hFGF-B, R3-IGF-1, Ascorbic Acid. |
| HASMC | Medium 231 supplemented with SMGS (Smooth |
| (Gibco, Invitrogen) | Muscle Growth Supplement), containing FBS |
| | (4.9% v/v), bFGF (2 ng/ml), hEGF (0.5 ng/ml), |
| | Heparin (5 ng/ml), Insulin (5 μg/ml), BSA |
| | (0.2 μg/ml), GA. |

All cultures were maintained in 25 cm² polystyrene tissue culture flasks in a 37° C., 5% CO₂/95% air environment, with cell culture media refreshed every other day. All cells were seeded at a density of 20,000˜30,000 cells/cm², as required by the specific experiment, and allowed to grow to 80-90% confluence before being harvested/passaged. Only cells from early passages (numbers 2-8) were utilized in experiments.

Primary cultures of mouse lung endothelial cells (MLEC) were isolated as previously described (Ackah, E. et al., J. Clin Invest., 2005, 115(8): 2119-27; Muto, A. et al., J. Exp. Med., 2011, 208(3): 561-575). Mouse lung endothelial cells (MLECs) were isolated from 3-week-old wild type mice. Briefly, mice were euthanized with an overdose of ketamine/xylazine and the lungs excised, minced, and digested with 0.1% collagenase in RPMI medium. The digest was homogenized by passing multiple times through a 14-gauge needle, filtered through a 150-μm tissue sieve, and the cell suspension was plated on 0.1% gelatin-coated dishes. Endothelial cells were then isolated by immunoselection with PECAM-1- and ICAM-2-conjugated magnetic beads. After immunoselection with magnetic beads, endothelial cells were immortalized with polyoma middle T-antigen. Isolated MLEC were maintained with EBM-2/EGM-2 MV SingleQuot® Kit Supplement & Growth Factors (Lonza) containing 15% fetal bovine serum. Cell proliferation in MLEC was measured at 24 and 72 hours after MMI-0100 treatment by direct cell counting after trypsin treatment.

MTS Cell Proliferation Assay

The CellTiter 96® AQᵤₑₒᵤₛ Non-Radioactive Cell Proliferation Assay (Promega) was used to assess drug effects on cell proliferation according to the manufacturer's instructions. Briefly, HAECs and HASMCs from early passages were grown to 80-90% confluence in 25 cm² tissue culture flasks in a 37° C./5% CO₂ incubator prior to harvest. 200 μl of each type of cell suspension (at 20,000 cells/cm²) was seeded onto separate 96-well plates to yield an approximate 60% confluence per well. Cells were allowed to adhere to the plate surface overnight, followed by addition of 20 ng/ml of TNF-α to stimulate production of inflammatory agents. After a 4-6 hour incubation period, MMI-0100 peptide drug (YARAAARQARAKALARQLGVAA; SEQ ID NO: 1) was added and cells incubated for another 20-24 hours. Each well was then supplemented with 100 μl of fresh medium and 20 μl of CellTiter 96® AQᵤₑₒᵤₛ One Solution Reagent and incubated for an additional 1.5-2 hours prior to measuring absorbance of each well at 490 nm with a SoftMax®-equipped plate reader.

Cell Apoptosis Analysis

The apoptotic effect of MMI-0100 (YARAAARQARAKALARQLGVAA; SEQ ID NO: 1) on mouse lung endothelial cells (MLEC) was measured at 24 hours after MMI-0100 (YARAAARQARAKALARQLGVAA; SEQ ID NO: 1) treatment. MLEC were removed from the tissue culture plate by trypsin, and re-suspended at a concentration of 1.0×10⁶ cells/ml. Apoptotic cells were detected by AlexaFluor® 488 annexin V/Dead Cell Apoptosis Kit (Invitrogen) using flow cytometry sorting analysis.

ELISA for IL-6, IL-8, and MCP-1 Detection

Primary human coronary artery endothelial cells (HCAEC) were cultured and seeded onto a 96-well plate, using methods described in the MTS proliferation assay above. Cells were again stimulated with 20 ng/ml of TNF-α for 6 hours and then treated with MMI-0100 (SEQ ID NO: 1) for approximately 24 hours. Supernatants were then collected and analyzed for drug effect on inflammatory cytokines.

IL-6 (Cat #: 900-K16; Lot #: 0909016) and IL-8 (Cat #: 900-K18; Lot #: 0209018) ELISA kits (Peprotech; Rocky Hill, N.J.) were used to measure levels of these cytokines from HCAEC supernatants following treatment with MMI-0100 (YARAAARQARAKALARQLGVAA; SEQ ID NO: 1). The Human IL-6 ELISA development kit contains the key components required for the quantitative measurement of natural and/or recombinant hIL-6 in a sandwich ELISA format within the range of 32-2000 pg/ml. Human IL-8 ELISA development kit contains the key components required for the quantitative measurement of natural and/or recombinant hIL-8 in a sandwich ELISA format within the range of 16-1000 pg/ml.

Nine standards were prepared by following the manufacturer's protocol. Human IL-6 standard contains 1 μg of recombinant hIL-6, 2.2 mg bovine serum albumin (BSA), and 11.0 mg D-mannitol. According to the manufacture's protocol (Cat# 900-K16), human IL-6 standard was reconstituted in 1 ml of sterile water for a concentration of 1 μg/ml and diluted into nine working standards whose concentration ranges from 2 ng/ml to zero. Human IL-8 standard contains 1 μg of recombinant hIL-6, 2.2 mg bovine serum albumin (BSA), and 11.0 mg D-mannitol. According to the manufacture's protocol (Cat # 900-K18), the human IL-8 standard was reconstituted in 1 ml of sterile water for a concentration of 1 μg/ml and diluted into nine working standards whose concentration ranges from 1 ng/ml to zero.

Briefly, 10 μl of supernatant was diluted with 90 μl of diluents; 3 replicates of each sample were used. Data were collected at 405 nm with correction at 650 nm on a plate reader. Each plate was monitored for 1 hour with readings taken every 5 minutes. Concentrations of IL-6 and IL-8 in test samples were determined by extrapolating from a standard curve. Data are expressed as means±SEM.

Monocyte Chemotractic Protein-1 (MCP-1) production from mouse lung endothelial cells (MLEC) was analyzed using conditioned culture medium by Quantikine® Mouse CCL/JE/MCP-1 Immunoassay (R&D Systems) following the manufacturer's instructions. Briefly, the assay employs a quantitative sandwich enzyme immunoassay technique in which a monoclonal antibody specific for MCP-1 has been pre-coated onto a microplaste. Standards and samples are pipetted into the wells and any MCP-1 present is bound by the immobilized antibody. After washing away any unbound substances, an enzyme-linked polyclonal antibody specific for MCP-1 is added to the wells. Following a wash to remove any unbound antibody-enzyme reagent, a substrate solution is added to the wells and color develops in proportion to the amount of MCP-1 bound in the initial step. The color development is stopped and the intensity of the color is measured at 450 nm, with wavelength correction at 540 nm or 570 nm.

NO Analysis

To measure nitric oxide (NO) production, conditioned medium from mouse lung endothelial cells (MLEC) was examined at 24 hours after treatment with MMI-0100 (YARAAARQARAKALARQLGVAA; SEQ ID NO: 1). The medium was processed for the measurement of nitrite (NO₂⁻) by a NO-specific chemiluminescence analyzer (Sievers) as previously described (Muto, A. et al., J. Exp. Med., 2011, 208(3): 561-575, incorporated herein by reference in its entirety.).

Human Saphenous Vein

De-identified, discarded segments of human saphenous vein (HSV) were collected from consented patients undergoing coronary artery or peripheral vascular bypass surgeries. HSV segments were stored in a saline solution until the end of the surgical procedure, at which time they were placed in cold transplant harvest buffer (100 mM potassium lactobionate, 25 mM KH₂PO₄, 5 mM MgSO₄, 30 mM Raffinose, 5 mM Adenosine, 3 mM Glutathione, 1 mM Allopurinol, 50 g/L Hydroxyethyl starch, pH 7.4). The vessels were used within 24 hours of harvest. Using sterile technique, HSV segments were transferred to a 60-mm Petri dish under a sterile hood. The edges (0.5 mm) of each segment were removed with a blade and excess adventitial tissue and fat removed with minimal manipulation. HSV segments were cut into consecutive rings of approximately 1.0 mm in width to be utilized for organ culture or muscle bath experiments. Two rings from each segment were immediately fixed in 10% formalin at 37° C. for 30 min to obtain pre-culture intimal thickening measurements.

Effect of MMI-0100 (YARAAARQARAKALARQLGVAA; SEQ ID NO: 1) on Smooth Muscle Physiology

In preparation for testing vein segment functional viability, HSV rings were weighed and their lengths recorded. To focus on smooth muscle responses, the endothelium was mechanically denuded by rolling the luminal surface of each ring at the tip of a fine vascular forceps before suspension in a muscle bath containing a bicarbonate buffer (120 mM NaCl, 4.7 mM KCl, 1.0 mM MgSO₄, 1.0 mM NaH₂PO₄, 10 mM glucose, 1.5 mM CaCl₂, and 25 mM Na₂HCO₃, pH 7.4) equilibrated with 95% O₂ and 5% CO₂ at 37° C. The rings were stretched and the length progressively adjusted until maximal tension was obtained. Normalized reactivity was obtained by determining the passive length-tension relationship for each vessel segment. Rings were maintained at a resting tension of 1 g, which produces maximal responses to contractile agonists as previously determined, and equilibrated for 2 hours in buffer. Force measurements were obtained using a Radnoti Glass Technology (Monrovia, Calif.) force transducer (159901A) interfaced with a Powerlab data acquisition system and Chart software (AD Instruments, Colorado Springs, Colo.).

HSV rings were first contracted with 110 mM KCl (with equimolar replacement of NaCl in bicarbonate buffer) and the generated force was measured. 110 mM KCl causes membrane depolarization, leading to contraction of vessels containing functionally viable smooth muscle. After multiple KCl challenges, rings were washed and allowed to equilibrate in bicarbonate solution for 30 min, and then contracted with phenylephrine (PE, 10⁻⁷-10⁻⁶ M). Rings were relaxed with a cumulative log dose of sodium nitroprusside (SNP), a nitric oxide donor, and the force generated was recorded. All rings were again washed and equilibrated in buffer for 15 minutes. Rings were then incubated with either buffer alone or buffer plus 100 μM of MMI-0100 (YARAAARQARAKALARQLGVAA (SEQ ID NO: 1)) for 2 hours, followed by treatment with the same doses of PE and SNP, and the forces generated again recorded. Measured force was normalized for ring weight and length and percent relaxation was calculated; force generated with 10⁻⁶ M of PE was set as 0% relaxation.

Organ Culture

After viability was determined in the muscle bath, additional rings were cut and placed in 8-well chamber slides and maintained in RPMI 1640 medium supplemented with 30% FBS, 1% L-glutamine and 1% penicillin/streptomycin for 14 days at 37° C. in an atmosphere of 5% CO₂ in air. The rings were either untreated or treated with 10 μM, 50 μM or 100 μM of MMI-0100 peptide (YARAAARQARAKALARQLGVAA (SEQ ID NO: 1)). The culture medium with treatments was replaced every 2-3 days.

Vessel Morphology

After 14 days of organ culture, vein segments were fixed in 0.5 mL of 10% formalin at 37° C. for 30 minutes and embedded in paraffin for sectioning. Beginning at the mid-portion of each ring, 5 transverse sections, spaced 5 μm apart, were cut for each specimen. Sections were then stained with Verhoeff-van Gieson stain. Each section was examined using light microscopy (Carl Zeiss, Thornwood, N.Y.) and 6 radially parallel measurements of intimal and medial thickness were randomly taken from each section (total of 6-12 measurements per ring). Intima was defined as tissue on the luminal side of the internal elastic lamina or the chaotic organization of the cells contained within it, whereas the medial layer was contained between the intimal layer and the external elastic lamina. Intimal and medial thickening was measured for each section at 5× magnification with the microscope's computerized image analysis system.

Mouse Vein Graft Model

12-week-old C57BL/6 wild type mice (Harlan) were used for all experiments, as previously described (Muto, A. et al., J. Exp. Med., 2011, 208(3): 561-575, incorporated herein by reference in its entirety). To obtain veins, an approximately 2.0 mm segment of the intrathoracic inferior vena cava was isolated and excised. Prior to implantation, the vein was treated ex vivo with 100 μM of MMI-0100 (YARAAARQARAKALARQLGVAA (SEQ ID NO: 1)) peptide solution, or control PBS solution, for 20 minutes at room temperature.

To implant the vein graft, a midline incision was made in the abdomen of a recipient mouse and the infrarenal abdominal aorta was exposed. The abdominal aorta was temporarily occluded with atraumatic micro-clamps and a segment corresponding to the length of the vein graft was excised. The vein was sutured into the arterial circulation using 10-0 nylon in continuous fashion.

Vein grafts were followed postoperatively using the Vevo770® High-Resolution Imaging System (VisualSonics, Toronto, Canada), with weekly measurements of graft wall thickness. At 28 days after surgery, mice were sacrificed to allow explantation of the vein graft. Tissue was either frozen with RNA stabilization reagent (Qiagen) or explanted for paraffin embedding after circulatory flushing with ice-cold PBS followed by 4% paraformaldehyde perfusion-fixation. Vein graft wall thickness, lumen diameter, and outer wall diameter (elastic lamina) were measured in elastin-stained sections using computer morphometry (ImageJ).

Immunohistochemistry

Vein graft samples were fixed as described above and harvested for histology. Specimens were embedded in paraffin and cut in cross section (5 μm). Hematoxylin & Eosin, Masson trichrome, and van Gieson elastin staining were performed for all samples. Cells were cultured on gelatin-coated cover slips and fixed with methanol.

All sections were treated for antigen retrieval using 10 mmol/L citrate buffer (pH 6.0) prior to boiling or proteinase K (20 μg/ml) treatment, at room temperature, for 10-15 minutes. Immunohistochemical detection was performed using DAB as well as NovaRED® substrate (Vector). Sections were counterstained with Mayer's Hematoxylin.

Statistics

Statistical analysis was performed with one-way ANOVA followed by Tukey test to compare experimental groups. Analyses were done with OriginPro 8 software (Originlab, Northampton, Mass.) or GraphPad software (La Jolla, Calif.). Statistical significance was accepted within a 95% confidence limit. Results are presented as arithmetic mean±SEM graphically.

### II. Results

### Example 1

### Development and Test of MMI-0100 (YARAAARQARAKALARQLGVAA; SEQ ID NO: 1)

A cell-penetrating peptide inhibitor of MK2 has been developed and optimized to promote its cellular uptake.

Peptide Synthesis and Purification

Peptide Synthesis

The MK2 inhibitor peptide YARAAARQARAKALARQLGVAA (MMI-0100; SEQ ID NO: 1) and its functional equivalents were synthesized on Rink-amide or Knorr-amide resin (Synbiosci Corp.) using standard FMOC chemistry on a Symphony® Peptide Synthesizer (Protein Technologies, Inc). The coupling reagent for the amino acids (Synbiosci Corp.) was 2-(1H-Benzotriazol-1-yl)-1,1,3,3-Tetramethylruonium Hexafluorophosphate (HBTU)/N-Methylmorhorline (NMM). Following synthesis, the peptide was cleaved from the resin with a trifluoroacetic acid-based cocktail, precipitated in ether, and recovered by centrifugation. The recovered peptide was dried in vacuo, resuspended in MilliQ® purified water, and purified using an FPLC (ÄKTA Explorer, GE Healthcare) equipped with a 22/250 C18 prep-scale column (Grace Davidson). An acetonitrile gradient with a constant concentration of either 0.1% trifluoroacetic acid or 0.1% acetic acid was used to achieve purification. Desired molecular weight was confirmed by time-of-flight MALDI mass spectrometry using a 4800 Plus MALDI TOF/TOF™ Analyzer (Applied Biosystems).

Radiometric IC₅₀ and Kinase Activity Determination

A commercial radiometric assay service was used to test the specificity and potency of MMI-0100 (YARAAARQARAKALARQLGVAA; SEQ ID NO: 1) by measuring its half maximal inhibitory concentrations (IC₅₀). This quantitative assay measures how much MK2 inhibitor (MMI-0100; YARAAARQARAKALARQLGVAA (SEQ ID NO: 1)) is needed to inhibit 50% of a given biological process or component of a process (i.e., an enzyme, cell, or cell receptor). Specifically, in these assays, a positively charged substrate is phosphorylated with a radiolabeled phosphate group from an ATP if the kinase is not inhibited by an inhibitor peptide. The positively charged substrate then is attracted to a negatively charged filter membrane, quantified with a scintillation counter, and compared to a 100% activity control. ATP concentrations within 15 μM of the apparent Kₘ for ATP were chosen since an ATP concentration near the Kₘ may allow for the kinases to have the same relative amount of phosphorylation activity. The individual conditions for each radiometric assay are as follows.

For the MK2 radiometric assay, 5-10 mU of MK2 was incubated with 50 mM Na b-glycerophosphate pH 7.5, 0.1 mM EGTA, 30 μM KKLNRTLSVA (SEQ ID NO: 34; a substrate peptide for MK2), 10 mM MgAcetate, and 10 μM of [y-³³P-ATP] in a final reaction volume of 25 μl. The reaction was initiated by adding the MgATP mix. After incubating for 40 minutes at room temperature, the reaction was stopped by adding 5 μl of a 3% phosphoric acid solution. 10 μl of the reaction was then spotted onto a P30 filtermat, washed three times for 5 minutes in 75 mM phosphoric acid and once in methanol. The washed P30 filtermat was dried and its radioactivity was measured by scintillation counter.

For the JNK (c-Jun N-terminal Kinase) radiometric assay, 5-10 mU of JNK was incubated with 50 mM Tris pH 7.5, 0.1 mM EGTA, 0.1% b-mercaptoethanol, 3 μM ATF2 (a substrate for JNK), 10 mM MgAcetate, and 10 μM of [y-³³P-ATP] in the final reaction volume of 25 μl. The reaction was initiated by adding the MgATP mix. After incubating for 40 minutes at room temperature, the reaction was stopped by adding 5 μl of a 3% phosphoric acid solution. 10 μl of the reaction was then spotted onto a P30 filtermat and washed three times for 5 minutes in 75 mM phosphoric acid and once in methanol. The washed P30 filtermat was dried and its radioactivity was measured by scintillation counter.

For the p38 MAPK (p38 Mitogen-Activated Protein Kinase) radiometric assay, 5-10 mU of p38 MAPK was incubated with 25 mM Tris pH 7.5, 0.02 mM EGTA, 0.33 mg/mL myelin basic protein (a substrate for p38 MAPK), 10 mM MgAcetate, and 10 μM of [y-³³P-ATP] in the final reaction volume of 25 μl. The reaction was initiated by adding the MgATP mix. After incubating for 40 minutes at room temperature, the reaction was stopped by adding 5 μl of a 3% phosphoric acid solution. 10 μl of the reaction was then spotted onto a P30 filtermat, and washed three times for 5 minutes in 75 mM phosphoric acid and once in methanol. The washed P30 filtermat was dried and its radioactivity was measured by scintillation counter.

For the MKK4 (Mitogen-Activated Protein Kinase Kinase 4) radiometric assay, 1-5 mU of MKK4 was incubated with 50 mM Tris pH 7.5, 0.1 mM EGTA, 0.1% b-mercaptoethanol, 0.1 mM Na₃VO₄, 2 μM inactive JNK1 (a substrate for MKK4), 10 mM MgAcetate, and 10 μM of cold [y-³³P-ATP] in the final reaction volume of 25 μl. The reaction was initiated by the addition of the MgATP. After incubation for 40 minutes at room temperature, 5 μl of the incubation mix was used to initiate the JNK radiometric assay described above.

For the MKK6 (Mitogen-Activated Protein Kinase Kinase) radiometric assay, 1-5 mU of MKK6 was incubated with 50 mM Tris pH 7.5, 0.1 mM EGTA, 0.1% b-mercaptoethanol, 0.1 mM Na₃VO₄, 1 mg/mL BSA, 1 μM inactive p38MAPK (a substrate for MKK6), 10 mM MgAcetate and 10 μM of cold [y-³³P-ATP] in the final reaction volume of 25 μl. The reaction was initiated by adding the MgATP. After incubation for 40 minutes at room temperature, 5 μl of the incubation mix was used to initiate the p38MAPK radiometric assay described above.

For the MEK1 (Meiosis-specific serine/threonine protein kinase) radiometric assay, 1-5 mU of MEK1 was incubated with 50 mM Tris pH 7.5, 0.2 mM EGTA, 0.1% b-mercaptoethanol, 0.01% Brij-35, 1 μM, inactive MAPK2 (a substrate for MEK1), 10 mM MgAcetate and 10 μM of cold [y-³³P-ATP] in the final reaction volume of 25 μl. The reaction was initiated by adding the MgATP. After incubation for 40 minutes at room temperature, 5 μl of this incubation mix was used to initiate the MAPK2 radiometric assay whose reaction condition was the same as the condition for p38 MAPK assay described above.

For the PRAK (p38-Regulated/Activated Protein Kinase) radiometric assay, 5-10 mU of PRAK was incubated with 50 mM Na b-glycerophosphate pH 7.5, 0.1 mM EGTA, 30 μM KKLRRTLSVA (SEQ ID NO: 35; a substrate peptide for PRAK), 10 mM MgAcetate and 10 μM of [y-³³P-ATP] in the final reaction volume of 25 μl. The reaction was initiated by adding the MgATP mix. After incubating for 40 minutes at room temperature, the reaction was stopped by adding 5 μl of a 3% phosphoric acid solution. 10 μl of the reaction was then spotted onto a P30 filtermat and washed three times for 5 minutes in 50 mM phosphoric acid and once in methanol prior to drying and scintillation counting.

IC₅₀ values for inhibitor peptides were determined using Millipore's IC₅₀ Profiler Express service. The IC₅₀ value was estimated from a 10-point curve of one-half log dilutions. In peptides that were tested for specificity, the concentration that inhibited approximately 95% of MK2 activity was chosen to profile against a battery of kinases related to MK2, cell viability, or human disease from Millipore's KinaseProfiler service. In both assays, compounds were supplied in DMSO. Every kinase activity measurement was conducted in duplicate.

MMI-0100 (YARAAARQARAKALARQLGVAA; SEQ ID NO: 1) comprising a protein transduction domain (PTD; YARAAARQARA; SEQ ID NO: 26) and a therapeutic domain (KALARQLGVAA; SEQ ID NO: 2) exhibited enhanced specificity and activity in inhibiting MK2 kinase. Moreover, MMI-0100 (YARAAARQARAKALARQLGVAA; SEQ ID NO: 1) did not show any evidence of toxicity, tissue erosion, or necrosis. The results show that MMI-0100 (YARAAARQARAKALARQLGVAA; SEQ ID NO: 1) is relatively specific for MK2, as compared to other kinases in the MAPK family and within the p38 signaling cascade, at the concentration of MMI-0100 (YARAAARQARAKALARQLGVAA; SEQ ID NO: 1) that inhibit >95% of MK2 activity (Table 2). Furthermore, because MMI-0100 (YARAAARQARAKALARQLGVAA; SEQ ID NO: 1) functions downstream of p38, its activity was more specific than p38MAPK inhibitors, affecting fewer intracellular signaling cascades.

TABLE 2
Specificity of the MMI-0100
(YARAAARQARAKALARQLGVAA;
SEQ ID NO: 1; 100 μM) peptide

| Kinase (100 μM) | Percent Activity |
|---|---|
| MK2 | <5 |
| JNK | 92 |
| MKK4 | 90 |
| MKK6 | 50 |
| MEK1 | 68 |
| PRAK | 100 |
| P38MAPK | 60 |

In order to determine the level of specificity for inhibiting MK2, the effect of MMI-0100 (YARAAARQARAKALARQLGVAA; SEQ ID NO: 1) on other kinases within the MAPK family and within the p38MAPK pathway was evaluated at concentrations of MMI-0100 (YARAAARQARAKALARQLGVAA; SEQ ID NO: 1) that inhibit greater than 95% of MK2 activity in vitro. Table 2 shows the level of kinase inhibition of MMI-0100 (YARAAARQARAKALARQLGVAA; SEQ ID NO: 1) that inhibit greater than 95% of MK2 activity in vitro. The data show that inhibition of other kinases by MMI-0100 (YARAAARQARAKALARQLGVAA; SEQ ID NO: 1) is significantly lower than that of MK2 by MMI-0100 (YARAAARQARAKALARQLGVAA; SEQ ID NO: 1). Although MKK6 retained 50% of its activity, there was no reduction in MKK3 activity (data not shown). Without being limited by theory, the reduced activity of MKK6 by MMI-0100 (YARAAARQARAKALARQLGVAA; SEQ ID NO: 1) is likely to be compensated by MKK3 in cells, since MKK3 has functional redundancy with MKK6. Thus, it is anticipated that the effects of MMI-0100 (YARAAARQARAKALARQLGVAA; SEQ ID NO: 1) are mostly achieved through inhibition of MK2, and that as a result, MMI-0100 (YARAAARQARAKALARQLGVAA; SEQ ID NO: 1) would induce fewer side effects when used as a therapeutic.

### Example 2

### MMI-0100 (YARAAARQARAKALARQLGVAA; SEQ ID NO: 1) Induces Minimal Cell Proliferation

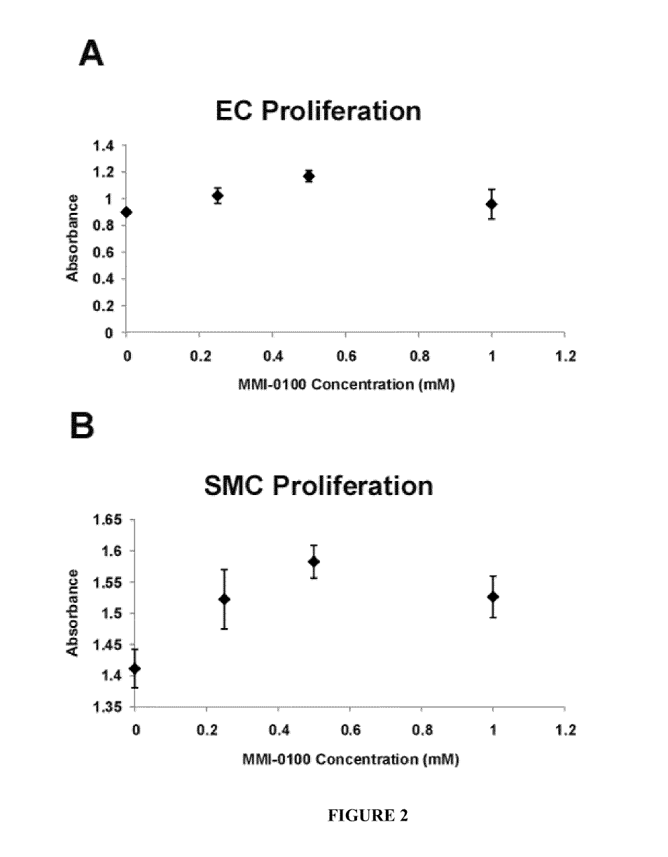

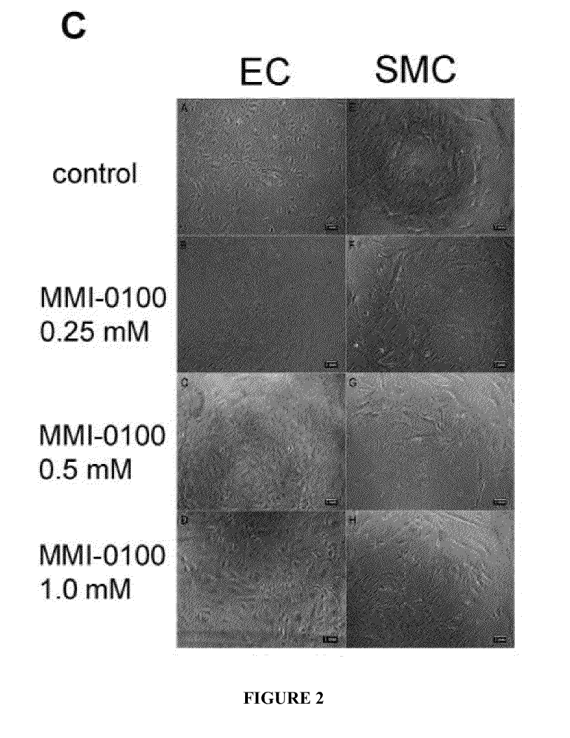

In order to determine the effect of pharmacological doses of MMI-0100 (YARAAARQARAKALARQLGVAA; SEQ ID NO: 1) on human endothelial cell (EC) and smooth muscle cell (SMC) proliferation, human EC and SMC cultures were treated with three concentrations (0.25 mM, 0.5 mM, and 1 mM) of MMI-0100 (YARAAARQARAKALARQLGVAA (SEQ ID NO: 1)) following pre-treatment with TNF-α. Both 0.25 mM and 0.5 mM concentrations of MMI-0100 (YARAAARQARAKALARQLGVAA; SEQ ID NO: 1) slightly increased cell proliferation in both cell types compared to control cells treated with 20 ng/ml TNF-α alone (maximum with 0.5 mM: 30% and 12% increases in EC and SMC cultures, respectively; FIG. 2, A-B). However, while the 1 mM MMI-0100 (YARAAARQARAKALARQLGVAA; SEQ ID NO: 1) treatment also increased both EC (11%) and SMC (7%) proliferation as compared to control, this response was not as robust as that induced by treatment with 0.5 mM MMI-0100 (YARAAARQARAKALARQLGVAA; SEQ ID NO: 1) (FIG. 2, A-B). Phase contrast images of EC and SMC treated with MMI-0100 (YARAAARQARAKALARQLGVAA; SEQ ID NO: 1) for 24 hours showed no obvious morphological changes as compared to control cells (FIG. 2C).

### Example 3

### Dose-Dependent Inhibition of TNF-α and IL-1β Expression by MMI-0100 (YARAAARQARAKALARQLGVAA; SEQ ID NO: 1) In Vitro

Lipopolysaccharide (LPS) is a compound with both lipid and carbohydrate components, derived from the cell wall of gram-negative bacteria. In vivo, infection of gram negative bacteria releases LPS into the blood stream, which activates monocytes. In response, the activated monocytes secret various inflammatory mediators, e.g., Tumor Necrosis Factor-alpha (TNF-α) and Interleukin-6 (IL-6), to combat the infection. Phorbol 12-myristate 13-acetate (PMA) is a compound that activates a wide variety of cell types that may contribute to acute inflammation. Particularly, PMA is known to activate human monocytic cells (THP-1 cells) in vitro. Thus, an inflammatory cellular response (e.g., release of inflammatory cytokines) can be induced in vitro by activating THP-1 cells with PMA and subsequently treating the activated THP-1 cells with LPS.

The ability of MMI-0100 (YARAAARQARAKALARQLGVAA; SEQ ID NO: 1) to inhibit the production of inflammatory cytokines (TNF-α and IL-1β) in vitro was examined using THP-1 cells. Briefly, THP-1 cells were activated with PMA for 24 hours, and treated subsequently with 10 μg of LPS. The amount of inflammatory cytokines (TNF-α and IL-1β, pg/ml) produced in the medium in the presence or absence of increasing concentrations (1 μM, 3 μM, 10 μM, and 30 μM) of MMI-0100 (YARAAARQARAKALARQLGVAA; SEQ ID NO: 1) was determined by cytokine specific enzyme-linked immunosorbent assay (ELISA). Analysis of inflammatory cytokines (TNF-α and IL-1β) was performed with cytokine-specific ELISA kit (PeproTech, Inc., Rocky Hill, N.J.). The cytokine-specific ELISA employs highly-purified anti-cytokine antibodies (capture antibodies) that are noncovalently adsorbed (“coated”) onto plastic microwell plates. After washings, the immobilized antibodies capture specifically soluble cytokine proteins present in samples applied to the plate. After washing away unbound material, the captured cytokine proteins are detected by biotin-conjugated anti-cytokine antibodies (detection antibodies) followed by an enzyme-labeled avidin or streptavidin reporter. Following addition of a chromogenic (color-developing) substrate-containing solution, the level of colored product generated by the bound, enzyme-linked, detection reagents can be measured spectrophotometrically using an ELISA-plate reader at an appropriate optical density.

In order to prepare plates for ELISA, capture antibodies (anti-TNF-α or anti-IL-1β antibodies) were diluted with PBS to a concentration of 1 μg/ml. 100 μl of each diluted antibody was added immediately to each well of the ELISA plate. The ELISA plate was sealed and incubated overnight at room temperature. On the following day, wells were aspirated and washed 4 times using 300 μl of wash buffer per well. After the last wash, the plate was inverted to remove residual buffer, blotted on paper towels, and incubated with 300 μl of blocking buffer for at least 1 hour at room temperature. After incubation, blocking buffer was aspirated and washed 4 times.

The standard was diluted from 2 ng/ml to zero in diluent, and 100 μl of the standard or sample was added immediately to each well in triplicate and incubated at room temperature for at least 2 hours. For detection, each well was aspirated and washed 4 times. 100 μl of the diluted detection antibody (0.5 μg/ml) then was added to each well and incubated at room temperature for 2 hours. Plates were washed 4 times and 100 μl of avidin-HRP conjugate (1:2000) was added and incubated for 30 min at room temperature for color development. The level of colored product generated by the bound, enzyme-linked, detection reagents was measured spectrophotometrically using an ELISA-plate reader at an appropriate optical density.

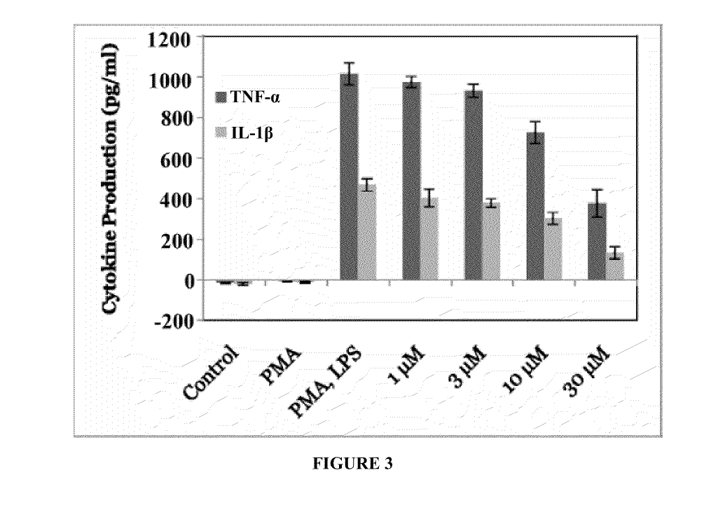

The data, shown in FIG. 3, show that MMI-0100 (YARAAARQARAKALARQLGVAA; SEQ ID NO: 1) inhibits the production of pro-inflammatory cytokines (i.e., TNF-α and IL-1β) in a dose-dependent manner. As expected, PMA treatment followed by LPS treatment increased the levels of inflammatory cytokines secreted by THP-1 cells, to about 1000 pg/ml (TNF-α) and to about 450 pg/ml (IL-1β), respectively. In contrast, treatment of THP-1 cells with 30 μM of MMI-0100 (YARAAARQARAKALARQLGVAA; SEQ ID NO: 1) significantly decreased the cytokine levels in the medium to about 300 pg/ml (TNF-α) and to about 100 pg/ml (IL-1β), respectively. These data suggest that MMI-0100 (YARAAARQARAKALARQLGVAA; SEQ ID NO: 1) is effective to suppress the production of pro-inflammatory cytokines (TNF-α and IL-1β in vitro.

### Example 4

### MMI-0100 (YARAAARQARAKALARQLGVAA; SEQ ID NO: 1) Inhibits the Production of Interleukin-6 (IL-6) in Mesothelial Cells

Interleukin-6 (IL-6) is a multifunctional cytokine whose major actions include enhancement of immunoglobulin synthesis, activation of T cells, and modulation of acute-phase protein synthesis. Many different types of cells are known to produce IL-6, including monocytes, macrophages, endothelial cells, and fibroblasts, and expression of the IL-6 gene in these cells is known to be regulated by a variety of inducers. Interleukin-1 (IL-1) and tumor necrosis factor-alpha (TNF-α) are two key known inducers of IL-6 gene expression. Other inducers include activators of protein kinase C, calcium ionophore A23187, and various agents causing elevation of intracellular cyclic AMP (cAMP) levels.

Tumor necrosis factor (TNF, also referred as TNF-α) is a cytokine involved in systemic inflammation and is a member of a group of cytokines that stimulate the acute phase reaction. Studies have shown that TNF-α induces expression of IL-6 via three distinct signaling pathways inside the cell, i.e., 1) NF-κB pathway 2) MAPK pathway, and 3) death signaling pathway.

In order to examine the efficacy of MMI-0100 (YARAAARQARAKALARQLGVAA; SEQ ID NO: 1) in inhibiting the production of inflammatory cytokines, an inflammatory response was triggered in vitro by applying TNF-α to immortalized human pleural mesothelial cells (ATCC CRL-9444). A mesothelial cell is a cell type that forms a monolayer of specialized pavement-like cells (mesothelium) that lines the body's abdominal cavities and internal organs. The primary function of this layer, the mesothelium, is to provide a slippery, non-adhesive, and protective surface. Mesothelial cells also are involved in transport of fluid and cells across the peritoneal (abdominal) cavities, antigen presentation, inflammation, tissue repair, coagulation, and tumor cell adhesion. It is well known that mesothelial cells play important role in the peritoneal inflammatory response. In response to bacterial products and macrophage-derived inflammatory cytokines (e.g., TNF-α), mesothelial cells produce Interleukin-1 (IL-1), Interleukin-6 (IL-6), and Interleukin-8 (IL-8), thus amplifying the inflammatory signals and recruiting leukocytes into the infected abdominal cavity.

In addition, the efficacy and specificity of MMI-0100 (YARAAARQARAKALARQLGVAA; SEQ ID NO: 1) also was compared with Rottlerin, a known small molecule inhibitor of MK2. Rottlerin was developed originally as a protein kinase C-delta (PKC-δ) competitive inhibitor, but later was found to inhibit MK2 and p38 regulated protein kinase (PRAK) more effectively than PKC-δ. In addition, unlike the peptide-based inhibitors of MK2 used in these studies, Rottlerin (IC₅₀=5 μM) is known to act through an ATP competitive mechanism.

Expression of inflammatory cytokines was induced by applying 1 or 10 ng/ml of TNF-α to cultured mesothelial cells. Briefly, immortalized human pleural mesothelial cells (ATCC CRL-9444) were grown in Medium199 with Earle's BSS, 0.75 mM L-glutamine (Mediatech Inc., Manassas, Va.), 1.25 g/l sodium bicarbonate (Sigma), 3.3 nM epidermal growth factor (EGF) (MBL International, Woburn, Mass.), 40 nM hydrocortisone (Sigma), 870 nM insulin (MBL International), 20 mM HEPES (Sigma), trace elements mixture B (Mediatech Inc., Waltham, Mass.), 10% fetal bovine serum (FBS) (Hyclone), and 1% penicillin/streptomycin (Mediatech Inc., Waltham, Mass.). The doses of TNF-α and time intervals of TNF application that significantly upregulate IL-6 expression were determined as follows. Human mesothelial cells were treated with two different concentrations (1 and 10 ng/mL) of TNF-α for 2, 6, 12, and 24 hours (data not shown). Overall, TNF-α increased IL-6 expression in a time- and dose-dependent manner. Without TNF-α stimulation, mesothelial cells made negligible amounts of IL-6. The 1 ng/mL dose of TNF-α did not significantly upregulate IL-6 expression above the untreated control. In subsequent experiments designed to determine whether the MMI-0100 peptide (YARAAARQARAKALARQLGVAA; SEQ ID NO: 1) could reduce TNF-α induced IL-6 expression, both 1 and 10 ng/mL of TNF-α were selected as stimuli. 10 ng/mL of TNF-α is regarded as more physiologically relevant for mesothelial cells.

Prior to cytokine and/or inhibitor treatment, cells were allowed to acclimate in serum-free media consisting of only Medium199 with Earle's BSS, 0.75 mM L-glutamine, 1.25 g/l sodium bicarbonate, 20 mM HEPES, trace elements mixture B (Mediatech Inc., Waltham, Mass.), and 1% penicillin/streptomycin (Mediatech Inc., Waltham, Mass.) for 24 hours. After the adapting period, cytokines (TNF-α and IL-1β with or without MMI-0100 peptide or Rottlerin (Tocris Bioscience, Ellisville, Mo.; IC₅₀=5 μM) were added to the media in order to compare the efficacy of MMI-0100 (YARAAARQARAKALARQLGVAA; SEQ ID NO: 1) with that of Rottlerin. Specifically, serum-starved mesothelial cells were treated with two different concentrations (1000 μM or 3000 μM) of MMI-0100 (YARAAARQARAKALARQLGVAA (SEQ ID NO: 1)) at three different time points (6 hours, 12 hours, and 24 hours) using two different concentrations of TNF-α (1 ng/ml or 10 ng/ml) or using 1 ng/ml of IL-1β. Average concentration of IL-6 (pg/ml per 105 cells) at each time was determined by cytokine-specific ELISA.

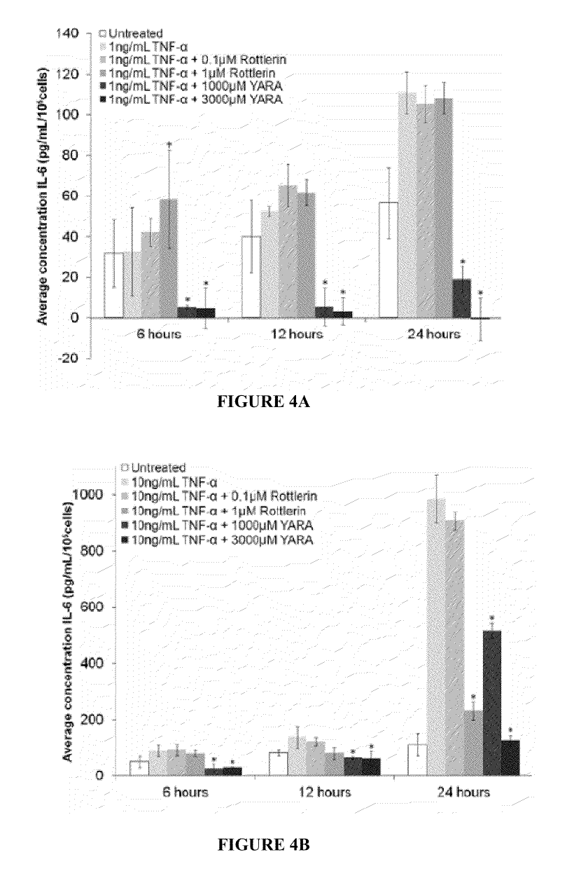

As shown in FIG. 4, both peptide concentrations (1000 μM or 3000 μM) of MMI-0100 (YARAAARQARAKALARQLGVAA; SEQ ID NO: 1) reduced IL-6 expression to the levels at or below the untreated baseline in mesothelial cells treated with 1 ng/mL of TNF-α (FIG. 4A). Rottlerin was ineffective in reducing IL-6 expression at this concentration of TNF-α. In fact, at 6 hrs, Rottlerin significantly increased IL-6 expression above that induced by TNF-α alone. When the concentration of TNF-α was increased to 10 ng/mL (FIG. 4B), the peptide was still effective in reducing IL-6 expression triggered by TNF-α, but Rottlerin became effective also at twenty-four hours. At every time point, the higher dose (3000 μM) of MMI-0100 (YARAAARQARAKALARQLGVAA; SEQ ID NO: 1) reduced IL-6 expression to the levels of the untreated baseline. Rottlerin reduced IL-6 expression only at a higher dose (1 μM) and only at twenty-four hours after treatment. These data suggest that MMI-0100 (YARAAARQARAKALARQLGVAA; SEQ ID NO: 1) may act more quickly and effectively than Rottlerin to inhibit IL-6 expression induced by TNF-α.

### Example 5

### MMI-100 (YARAAARQARAKALARQLGVAA; SEQ ID NO: 1) Reduces Interleukin-6 (IL-6) Expression in Endothelial Cells

The anti-inflammatory effect of pharmacological doses of MMI-0100 (YARAAARQARAKALARQLGVAA; SEQ ID NO: 1) was investigated further by assaying expression of Interleukin 6 (IL-6) and Interleukin-8 (IL-8) secreted by human coronary endothelial cells (HCAEC) following TNF-α stimulation. Briefly, HCAEC were seeded on a multi-well plate at a density of approximately 25,000 cells/cm². After a 6 hour incubation with TNF-α, MMI-0100 (YARAAARQARAKALARQLGVAA; SEQ ID NO: 1; 0.5 mM) was added to the culture medium. After 24 hours of drug treatment, supernatant from each well was collected and assayed for cytokine expression.

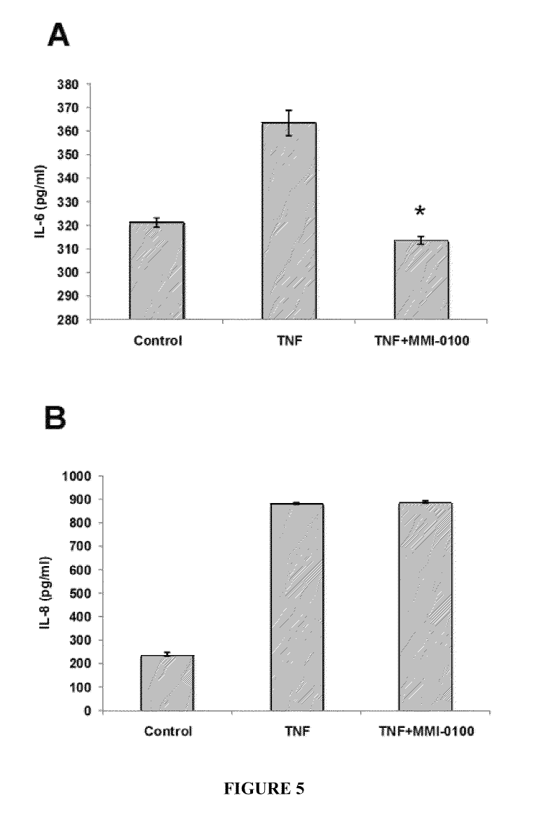

MMI-0100 (YARAAARQARAKALARQLGVAA; SEQ ID NO: 1) treatment reduced the level of TNF-α-induced IL-6 expression to that of the untreated control (FIG. 5A). However, MMI-0100 (YARAAARQARAKALARQLGVAA; SEQ ID NO: 1) had no effect on the level of TNF-α-induced IL-8 expression (FIG. 5B).

### Example 6

### Functional Contractility and Cellular Viability of the Human Saphenous Veins

Molecular Mechanisms of Smooth Muscle Contraction and Relaxation

The mechanisms of smooth muscle contraction and relaxation are summarized by R. Clinton Webb (Adv. Physiol. Educ. 27: 201-206 (2003)), which is incorporated herein by reference in its entirety. Sheets or layers of smooth muscle cells are contained in the walls of various organs and tubes in the body, including the blood vessels. The process of smooth muscle contraction requires the formation of crossbridges and sliding of thin (actin) filaments past thick (myosin) filaments as in striated muscle and cardiac muscle, but because it is not well organized (as are striated and cardiac muscle), smooth muscle contracts in all directions. When made to contract, the smooth muscle cells shorten, thereby varying the diameter of the vessel to regulate the flow of its contents.

In the body, the process of smooth muscle cell contraction is regulated principally by receptor and mechanical (stretch) activation of the contractile proteins myosin and actin, although a change in membrane potential, brought on by the firing of action potentials or by activation of stretch-dependent ion channels in the plasma membrane also can trigger contraction.

Extracellular calcium enters through L-type calcium channels, and intracellular calcium is released predominantly from intracellular stores in the sarcoplasmic reticulum. Phospholipase C activity, which is specific for the membrane lipid phosphatidylinositol 4,5-bisphosphate, is stimulated by binding of agonists (norepinephrine, angiotensin II, endothelin, etc.) to serpentine receptors, coupled to a heterotrimeric G protein. Phospholipase C catalyzes the formation of two potent second messengers: inositol trisphosphate (IP₃) and diacylglycerol (DG). The binding of IP3 to receptors on the sarcoplasmic reticulum results in the release of Ca²⁺ into the cytosol. DG, along with Ca²⁺, activates protein kinase C (PKC), which phosphorylates specific target proteins. There are several isozymes of PKC in smooth muscle, and each has a tissue-specific role (e.g., vascular, uterine, intestinal, etc.). In many cases, PKC has contraction-promoting effects such as phosphorylation of L-type Ca²⁺ channels or other proteins that regulate cross-bridge cycling. Phorbol esters, a group of synthetic compounds known to activate PKC, mimic the action of DG and cause contraction of smooth muscle. Finally, L-type Ca²⁺ channels (voltage-operated Ca²⁺ channels) in the membrane also open in response to membrane depolarization brought on by stretch of the smooth muscle cell.

Contractile activity in smooth muscle is determined primarily by the phosphorylation state of the myosin light chain. Calcium activated phosphorylation of the myosin light chain initiates smooth muscle contraction. The activator Ca²⁺ forms a complex with the acidic protein calmodulin, which in turn activates myosin light chain (MLC) kinase to phosphorylate the light chain of myosin.

In addition to the Ca²⁺-dependent activation of MLC kinase, the state of myosin light chain phosphorylation is further regulated by MLC phosphatase, which removes the high-energy phosphate from the light chain of myosin to promote smooth muscle relaxation. There are three subunits of MLC phosphatase: a 37-kDa catalytic subunit, a 20-kDa variable subunit, and a 110- to 130-kDa myosin-binding subunit. The myosin-binding subunit, when phosphorylated, inhibits the enzymatic activity of MLC phosphatase, allowing the light chain of myosin to remain phosphorylated, thereby promoting contraction. The small G protein RhoA and its downstream target Rho kinase play an important role in the regulation of MLC phosphatase activity. Rho kinase, a serine/threonine kinase, phosphorylates the myosin-binding subunit of MLC phosphatase, inhibiting its activity and thus promoting the phosphorylated state of the myosin light chain. Pharmacological inhibitors of Rho kinase, such as fasudil and Y-27632, block its activity by competing with the ATP-binding site on the enzyme. Rho kinase inhibition induces relaxation of isolated segments of smooth muscle contracted to many different agonists. In the intact animal, the pharmacological inhibitors of Rho kinase have been shown to cause relaxation of smooth muscle in arteries, resulting in a blood pressure-lowering effect. It is thought that receptors activate a heterotrimeric G protein that is coupled to RhoA/Rho kinase signaling via guanine nucleotide exchange factors (RhoGEFs). Because RhoGEFs facilitate activation of RhoA, they regulate the duration and intensity of signaling via heterotrimeric G protein receptor coupling. There are 70 RhoGEFs in the human genome, three of which have been identified in smooth muscle: PDZ-RhoGEF, LARG (leukemia-associated RhoGEF), and p115-RhoGEF.

Several recent studies suggest a role for additional regulators of MLC kinase and MLC phosphatase. Calmodulin-dependent protein kinase II promotes smooth muscle relaxation by decreasing the sensitivity of MLC kinase for Ca²⁺. Additionally, MLC phosphatase activity is stimulated by the 16-kDa protein telokin in phasic smooth muscle and is inhibited by a downstream mediator of DG/protein kinase C, CPI-17.

Smooth muscle relaxation occurs either as a result of removal of the contractile stimulus or by the direct action of a substance that stimulates inhibition of the contractile mechanism (e.g., atrial natriuretic factor is a vasodilator). The process of relaxation requires a decreased intracellular Ca²⁺ concentration and increased MLC phosphatase activity.

Several mechanisms involving the sarcoplasmic reticulum and the plasma membrane are implicated in the removal of cytosolic Ca²⁺. Ca²⁺ uptake into the sarcoplasmic reticulum is dependent on ATP hydrolysis. A sarcoplasmic reticular Ca²⁺, Mg²⁺-ATPase, when phosphorylated, binds two Ca²⁺ ions, which then are translocated to the luminal side of the sarcoplasmic reticulum and released. Mg²⁺, which binds to the catalytic site of the ATPase, is necessary for the enzyme to mediate the reaction. The sarcoplasmic reticular Ca²⁺, Mg²⁺-ATPase is inhibited by several different pharmacological agents: vanadate, thapsigargin, and cyclopiazonic acid. Sarcoplasmic reticular Ca²⁺-binding proteins also contribute to decreased intracellular Ca²⁺ levels. Recent studies have identified calsequestrin and calreticulin as sarcoplasmic reticular Ca²⁺-binding proteins in smooth muscle.

The plasma membrane also contains Ca²⁺, Mg²⁺-ATPases, providing an additional mechanism for reducing the concentration of activator Ca²⁺ in the cell. This enzyme differs from the sarcoplasmic reticular protein in that it has an autoinhibitory domain that can be bound by calmodulin, causing stimulation of the plasma membrane Ca²⁺ pump.

Na⁺/Ca²⁺ exchangers also are located on the plasma membrane and aid in decreasing intracellular Ca²⁺. This low-affinity antiporter is closely coupled to intracellular Ca²⁺ levels and can be inhibited by amiloride and quinidine.

Inhibition of receptor-operated and voltage-operated Ca²⁺ channels located in the plasma membrane also can elicit relaxation. Channel antagonists such as dihydropyridine, phenylalkylamines, and benzothiazepines bind to distinct receptors on the channel protein and inhibit Ca²⁺ entry in smooth muscle.

Isometric and Isotonic Muscle Contraction

The term “isometric contraction” as used herein refers to a muscle contraction in which the muscle is activated, but it is held at a constant length instead of being allowed to lengthen or shorten. Therefore, the force generated during an isometric contraction becomes dependent on the length of the muscle while contracting. On the other hand, if the muscle is allowed to shorten, for example, if only one end of the muscle is fixed and the muscle shortens with a constant load, the contraction is called isotonic contraction.

Smooth muscle in blood vessels maintains a partially isometric contraction where the force is held constant for an extended period of time, resulting in a particular blood pressure.

In vitro, the isometric contraction of smooth muscle can be induced by applying a depolarizing solution, for example, concentrated potassium chloride solution, to the muscle to be examined. The high concentration of potassium depolarizes the muscle cell membrane and opens voltage-gated calcium channels, resulting in an influx of extracellular calcium and activation of contractile machinery.

The human saphenous vein (HSV) is used most widely as a graft for coronary artery revascularization procedures. Studies have suggested that the harvested HSV is susceptible to vasospasm, and that the vasospasm ultimately leads to the death of smooth muscle cells in HSV grafts.

Therefore, the survival of smooth muscle in an HSV graft can be examined by measuring, the isometric contraction of harvested HSVs after surgery. Live cells in each harvested HSV can be quantified using an MTT live-dead cell assay. The MTT live-dead cell assay is a colorimetric assay in which cells are labeled with a staining solution and quantified by spectrophotometry. The assay utilizes yellow tetrazolium salts (MTT, chromogenic substrate) that are reduced by a mitochondrial enzyme in metabolically-active cells. The MTT salts in the staining solution become reduced into insoluble purple formazan crystals in live cells. Therefore, It is possible to quantify the number of purple-stained live cells using spectrophotometry.

Vein Segments with Lower Functional Contractile Responses Show Less Cellular Viability

In order to examine whether the survival of vascular smooth muscle cells in the harvested HSV depends on the functional contractility of the vessel, HSVs harvested from two patients (HSV 54 or HSV 55) were suspended between two wires, and their isometric contraction was measured in response to high potassium chloride (110 mM KCl, a concentration that depolarizes muscle) in a muscle bath (5 g). Cell survival of the vein segments was determined by staining the tissue and by quantifying the live cells via live-dead cell assay (methylthiazol tetrazolium, MTT) described above.

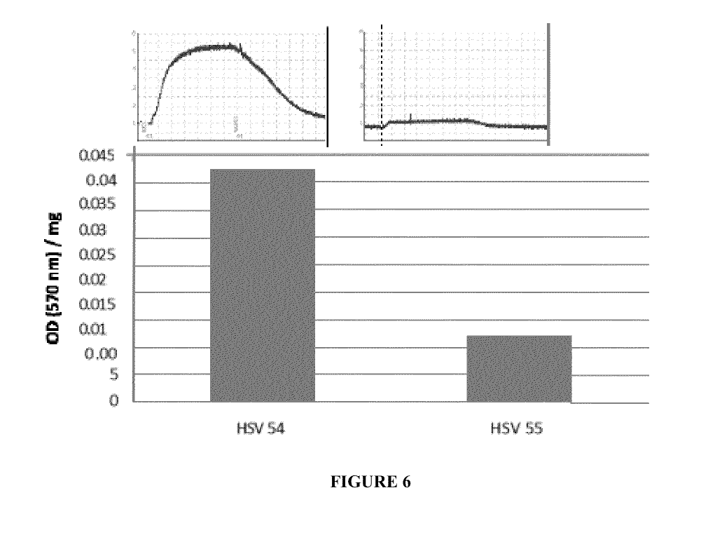

FIG. 6 shows representative tracings from two different human saphenous veins (HSV) harvested from two patients. As shown in FIG. 6, the live-dead cell assay (methylthiazol tetrazolium, MTT) revealed that vein segments that show lower functional contractile responses (upper panel) in the isometric contraction assay contained fewer live vascular smooth muscle cells (lower panel). HSV54 showed a robust contractile response to high potassium chloride (110 mM KCl, upper left panel), and had live cellular mitochondria-based on MTT staining (lower left panel). In contrast, HSV 55 generated minimal force in response to 110 mM KCl (upper right panel) and had fewer live cells (less MTT staining, lower right panel). This result suggests (i) that the vein harvest procedure itself leads to considerable injury to the conduit, and (ii) that maintaining functional contractility of HSV during/after harvest is important to ensure ultimate survival of the smooth muscle cells in the transplanted vascular graft.

### Example 7

### The MK2 Inhibitor YARAAARQARAKALARQLGVAA (MK2i; SEQ ID NO: 1) Enhances Sodium Nitroprusside (SNP)-Induced Relaxation of Human Saphenous Vein (HSV)

Sodium Nitroprusside (SNP) is a vasodilator that relaxes smooth muscles lining blood vessels. SNP breaks down in circulation to release nitric oxide (NO), which increases intracellular production of cGMP by activating guanylate cyclase in the vascular smooth muscle cells. The increased amount of cGMP in the cytoplasm drives calcium from the cytoplasm to the endoplasmic reticulum and reduces calcium available in the cytoplasm to bind with calmodulin, a key step required for smooth muscle contraction. As a result, vascular smooth muscle relaxes and vessels dilate.

The enhancing effect of MMI-0100 (YARAAARQARAKALARQLGVAA; SEQ ID NO: 1) on smooth muscle relaxation was examined by pre-treating harvested human saphenous vein (HSV) with MMI-0100 (YARAAARQARAKALARQLGVAA; SEQ ID NO: 1) before SNP treatment. Segments of human saphenous vein (HSV) were equilibrated in a muscle bath and pretreated with a control solution (0.01M phosphate-buffered solution, PBS) or MMI-0100 (YARAAARQARAKALARQLGVAA (SEQ ID NO: 1); 30 μM, 2 hrs), contracted with norepinephrine, and relaxed with increasing doses of sodium nitroprusside (SNP, 0-1 μM). Percentage of relaxation at 0, 0.01, 0.1, and 1.0 μM of SNP was measured.

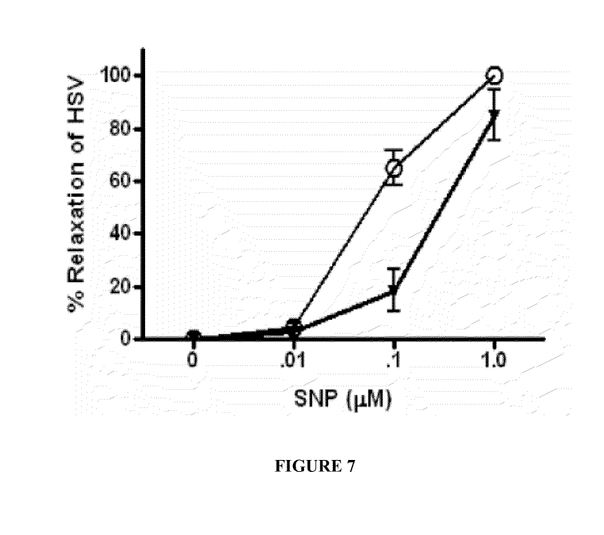

As shown in FIG. 7, HSV pretreated with MMI-0100 (YARAAARQARAKALARQLGVAA; SEQ ID: NO: 1) before SNP treatment (open circle) exhibited about 40% increase in relaxation compared to HSV pretreated with a control solution (closed reverse triangle), demonstrating that pretreatment of HSV with MMI-0100 (YARAAARQARAKALARQLGVAA; SEQ ID NO: 1) leads to a significant increase in HSV relaxation in response to sodium nitroprusside (SNP) (*=p<0.05 compared to control, n=3). These results also suggest that MMI-0100 (YARAAARQARAKALARQLGVAA; SEQ ID NO: 1) may be effective to treat vasospasm that occurs during vessel harvest, which is refractory to current vasodilator pharmacologic approaches.

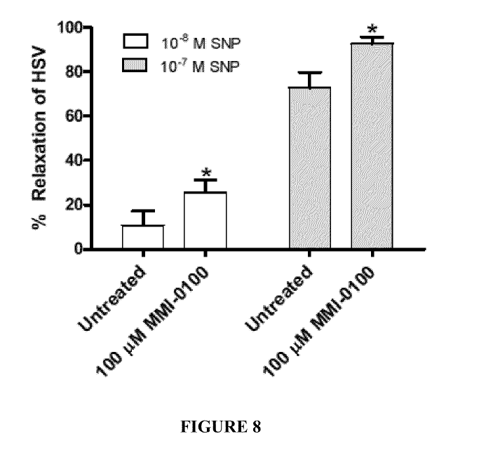

The direct role of MMI-0100 (YARAAARQARAKALARQLGVAA; SEQ ID NO: 1) on smooth muscle relaxation was examined further in a follow-up study. Specifically, human saphenous vein (HSV) rings were pre-treated with buffer or MMI-0100 (YARAAARQARAKALARQLGVAA; SEQ ID NO: 1; 100 μM); rings then were contracted with phenylephrine (10⁻⁶ M) and relaxed with sodium nitroprusside (10⁻⁸ M and 10⁻⁷ M; SNP). Pretreatment of HSV rings with MMI-0100 (YARAAARQARAKALARQLGVAA; SEQ ID NO: 1) led to a significant increase in relaxation (25.622±6.38 and 92.54±3.09 for 10⁻⁸ and 10⁻⁷ M SNP, respectively) when compared to untreated control (10.825±5.62 and 72.768±6.99 for 10⁻⁸ and 10⁻⁷ M SNP, respectively) (FIG. 8). There was no significant difference in relaxation response when HSV rings were pretreated with the control peptide (transduction domain, PTD peptide, 50 or 100 μM) when compared to the untreated control (data not shown).

### Example 8

### MMI-0100 (YARAAARQARAKALARQLGVAA; SEQ ID NO: 1) Reduces Intimal Hyperplasia in a Human Saphenous Vein Organ Culture Model

The standard procedure for the revascularization of ischemic heart tissue is a coronary artery bypass graft (CABG), and saphenous vein grafts have been used widely in CABG. However, the effectiveness of saphenous vein grafts has been hampered by accelerated intimal hyperplasia that develops within the vein conduit.

The well-established ex vivo culture of human saphenous vein was used as a model of the intimal hyperplasia that occurs in vivo. The procedure for culturing HSV ex vivo is well known in the art. Most protocols in the art use vessel rings that can be maintained under well-controlled hemodynamic conditions. For example, a vessel chamber is connected to a perfusion system where media from the reservoir is pumped at a set flow rate. The flow circuit is maintained in a humidified 5% CO₂ at 37° C. Under such condition, HSV can be maintained ex vivo for an extended period of time; however the cultured HSV usually develops intimal hyperplasia starting on or about 7 days post-operation.

The efficacy of MMI-0100 (YARAAARQARAKALARQLGVAA; SEQ ID NO: 1) in treating intimal hyperplasia was examined using this model. HSPB1 kinase inhibitor (r-HSPB1; SEQ ID NO: 36) was used as a control. HSPB1 kinase affects smooth muscle cell proliferation, smooth muscle cell migration, and extracellular matrix production by smooth muscle cells. Unlike MK2, HSPB1 kinase does not affect inflammation-induced intimal hyperplasia.

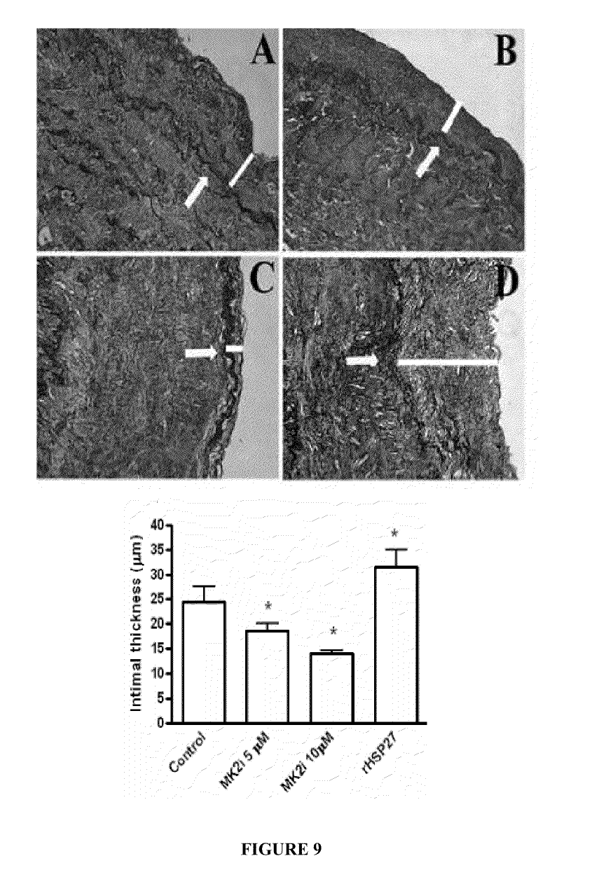

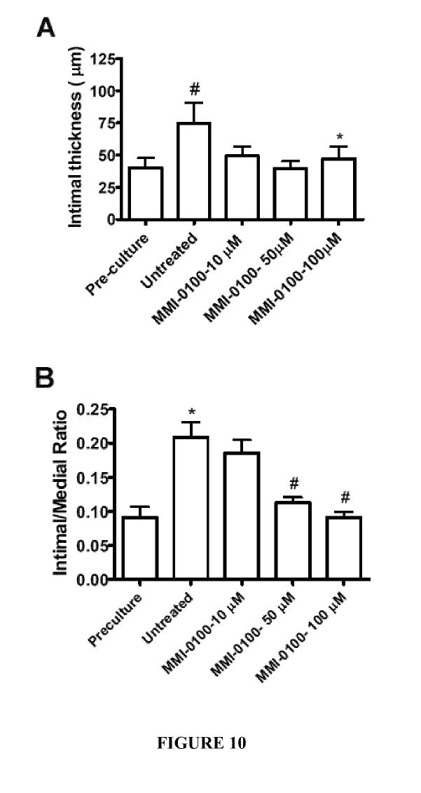

To this end, rings of human saphenous vein (HSV) were cultured in RPMI medium supplemented with L-glutamine (1%), penicillin/streptomycin (1%), and fetal bovine serum (FBS) (30%) at 5% CO₂ and 37° C. for 14 days. The rings were either left untreated, treated with either 5 μM or 10 μM of MMI-0100 (YARAAARQARAKALARQLGVAA (SEQ ID NO: 1), or treated with a cell-permeant HSPB1 kinase inhibitor peptide (r-HSPB1; SEQ ID NO: 36; 20 mM). After 14 days, the rings were fixed in formalin, sectioned and stained using Weigert's resorcin-fucsin. Morphometric analysis was performed to measure the mean intimal thickness. As shown in FIG. 9, in contrast to r-HSPB1 (SEQ ID NO: 36, labeled as “rHSP27”), MMI-0100 (YARAAARQARAKALARQLGVAA; SEQ ID NO: 1; labeled as “MK2i”) significantly reduced the mean intimal thickness of HSV in culture. The data show, for example, that the mean intimal thickness of HSV treated with MMI-0100; YARAAARQARAKALARQLGVAA; SEQ ID NO: 1) (10 μM) was 14 μm compared to that of HSV treated with rHSPB1, which was about 32 μm. These data suggest that MMI-0100 (YARAAARQARAKALARQLGVAA; SEQ ID NO: 1) is more effective than the inhibitor for the HSPB1 kinase to treat intimal hyperplasia.

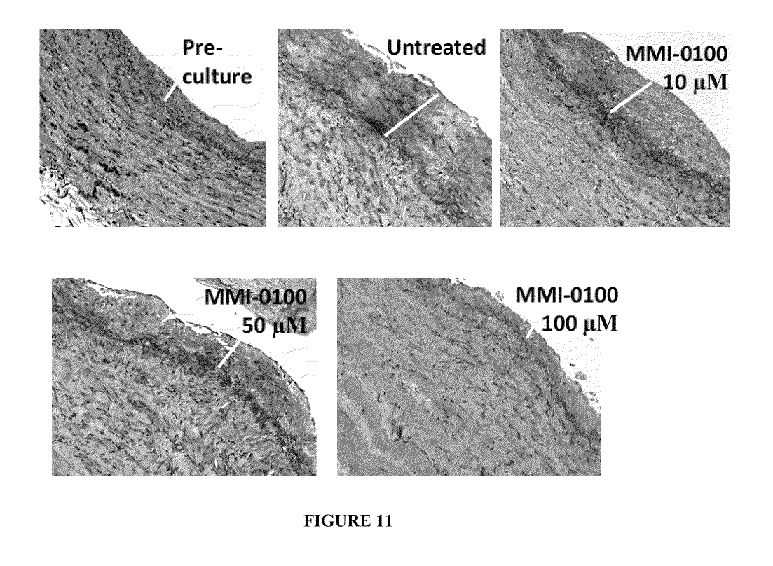

The effect of MMI-0100 (YARAAARQARAKALARQLGVAA; SEQ ID NO: 1) on development of intimal hyperplasia was further examined in a follow-up study in which intimal thickness of HSV in an organ culture model was measured in the presence of high serum and different concentrations (10-100 μM) of MMI-0100 (YARAAARQARAKALARQLGVAA; SEQ ID NO: 1). HSV were cultured for 14 days in 30% serum. All veins were deemed viable at the time of culture by adequate contraction with a phenylephrine challenge in a muscle bath. The average intimal thickness of pre-cultured vein segments was 43.7±7.8 μm. After culture, the average intimal thickness of the control was 81.6±17.3 μm. The average intimal thickness in the presence of 50 μM and 100 μM MMI-0100 (YARAAARQARAKALARQLGVAA; SEQ ID NO: 1) was 42.7±6.0 μm and 50.4±10.7 μm, respectively, with a significant reduction in intimal thickness (FIGS. 10A and 11). Measurement of the intima:media (I:M) ratio showed a greater reduction of the I:M ratio at the 100 μM concentration of MMI-0100 (YARAAARQARAKALARQLGVAA; SEQ ID NO: 1) (FIG. 10B).

### Example 9

### MMI-0100 Inhibits Intimal Hyperplasia in a Mouse Vein Graft Model

Vascular grafts are used widely for treatment of severe atherosclerosis during coronary artery bypass graft (CABG) surgery, a procedure often complicated by later occlusions of the graft vessel. The small caliber autogenous saphenous vein is used usually as a graft, but occlusion (stenosis) of the graft vein often occurs after bypass operation. Three pathological processes have shown to be responsible primarily for graft occlusion: thrombosis (early closure), intimal hyperplasia (a few months to a few years), and atherosclerosis (usually after 1 year).

The pathogenesis of vein graft atherosclerosis was extrapolated often from studies on spontaneous atherosclerosis in arteries. However, because the features of the lesions and the pathogenic processes of graft-induced atherosclerosis differ significantly from spontaneous atherosclerosis, appropriate animal models for vein grafts were needed to study the disease. Several animal models of vascular graft-induced atherosclerosis, e.g., vein bypass grafts in a primate or canine, manifesting lesions resembling human vascular graft arteriosclerosis have been developed to explore specific interventional issues. Among them, a mouse model has attracted researchers studying the molecular and cellular mechanisms of intimal hyperplasia, because many inbred mouse lines have been established already and the genetic map is defined relatively well. Using this model, it has become possible to understand the cellular and molecular mechanisms of vein graft atherosclerosis and, in particular, intimal hyperplasia. For example, studies have been reported that one of the earliest cellular events in new intimal formation in atherosclerosis is cell death, in which biomechanical stress is a critical factor. Mouse studies also have revealed i) that, after cell death, mononuclear cells infiltrate massively into the vessel wall; ii) that biomechanical stress directly stimulates expression of adhesion molecules and chemokines in endothelial and smooth muscle cells; iii) that dead cells induce inflammatory responses in the vessel wall; and iv) that vascular smooth muscle cells proliferate and differentiate during development of vascular graft atherosclerosis.

Although some studies have reported that the genetic background of a mouse model may influence the formation of atherosclerotic lesions in hyperlipidemia models (i.e., elevation of lipids, such as, cholesterol, cholesterol esters, estersphospholipids, and triglycerides, in the blood stream), accumulating data suggest that there are no significant differences between various genetic backgrounds, e.g., C57BL/6J and BALB/c, in either inflammatory responses or in the thickness of lesions in vascular graft-induced intimal hyperplasia mouse models, indicating that the vascular graft-induced model of intimal hyperplasia is less influenced by the genetic background of the mouse.

The protocol for preparing a mouse model of atherosclerosis is well known in the art. Typically, autologous (originating from the same animal) or isogeneic (originating from the same genetic composition) vessels of external jugular, or vena cava veins are end-to-end grafted into carotid arteries of C57BL/6J mice. Vessel wall thickening is observed usually as early as 1 week after surgery, and later the lumen of grafted veins is significantly narrowed due to the development of intimal hyperplasia.

The efficacy of MMI-0100 (YARAAARQARAKALARQLGVAA; SEQ ID NO: 1) in treating intimal hyperplasia in vivo was examined using a mouse model of vein bypass graft. Specifically, donor (C57BL/6J) mice were anesthetized and the intrathoracic inferior vena cava (IVC) was harvested. The IVC was soaked in a 100 μM solution of MMI-0100 (YARAAARQARAKALARQLGVAA; SEQ ID NO: 1) at room temperature for 20 minutes prior to implantation into a host (C57BL/6J) mouse to bypass the infrarenal aorta. Five treated and six untreated veins were transplanted for a total of 11 transplanted animals. Vein graft wall thickness was determined using a Vevo7700 ultrasound imaging system at each week.

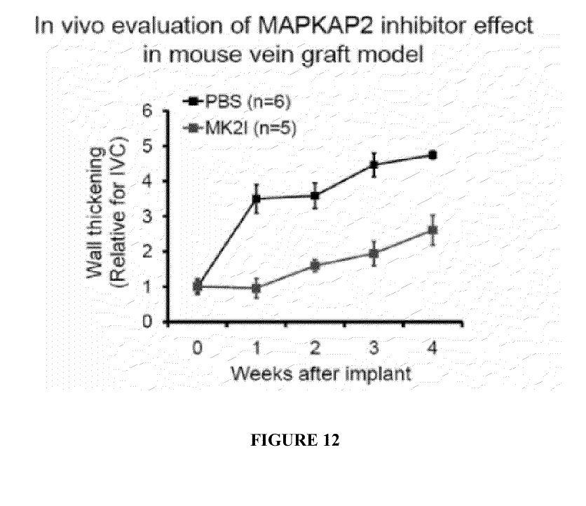

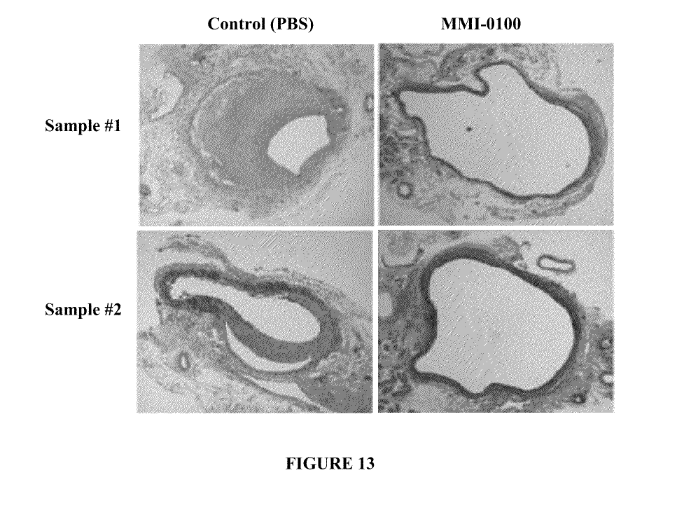

As shown in FIGS. 12 and 13, thickening of the grafted blood vessel wall in the control mouse was seen as early as one week after surgery, and the intimal thickening continued to progress every week. Four weeks after surgery, for example, the vein wall thickness of the grafted IVC in the control (PBS-treated) mouse reached almost five times the thickness of the originally harvested IVC. In contrast, 4 weeks after vascular graft, the grafted vessel wall of the mouse treated with MMI-0100 peptide (YARAAARQARAKALARQLGVAA; SEQ ID NO: 1) was significantly thinner (only about 2.5 times thicker than the originally harvested IVC) in vessel wall thickness than the control mouse treated with a control solution. These data demonstrate that MMI-0100 (YARAAARQARAKALARQLGVAA; SEQ ID NO: 1) and its functional equivalents can be used as an effective therapeutic agent in preventing vascular graft-induced intimal hyperplasia and in treating a vascular disease comprising intimal hyperplasia in vivo.

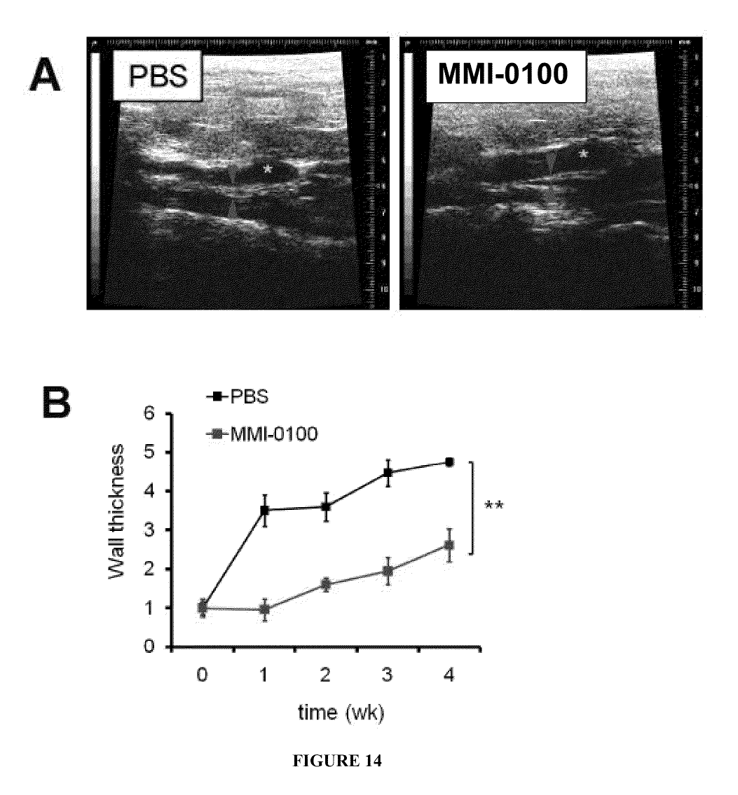

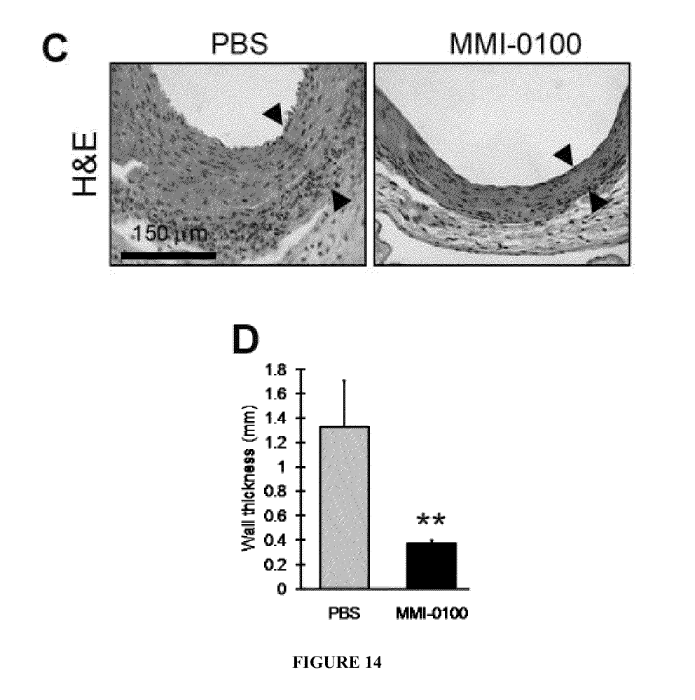

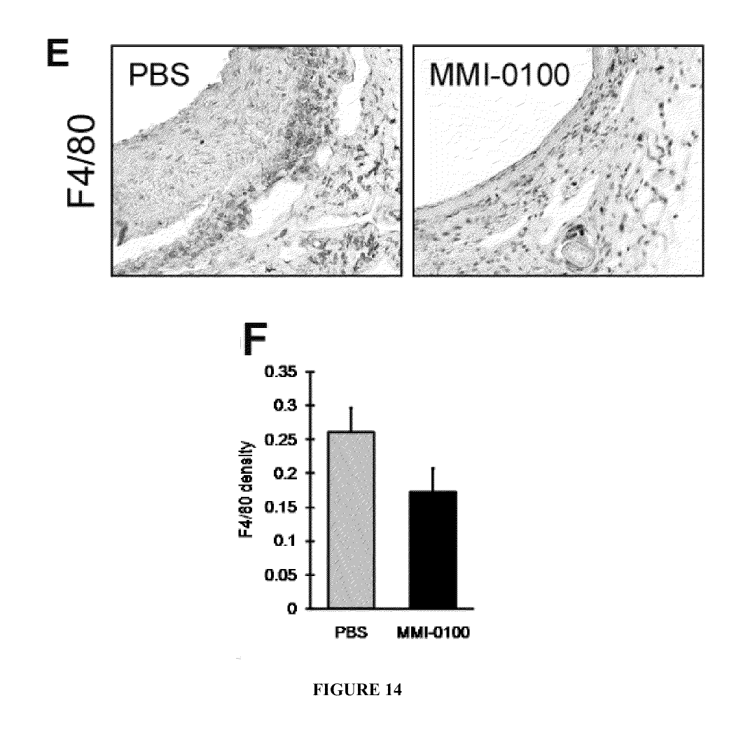

In order to further confirm the inhibitory effects of MMI-0100 (YARAAARQARAKALARQLGVAA; SEQ ID NO: 1) on intimal hyperplasia development in an ex vivo model, the effect of MMI-0100 (YARAAARQARAKALARQLGVAA; SEQ ID NO: 1) was investigated in a follow-up study using a mouse model of vein graft adaptation. To this end, vein grafts were treated with PBS or 100 μM of MMI-0100 (YARAAARQARAKALARQLGVAA; SEQ ID NO: 1) for 20 minutes prior to implantation; the thickness of the vein grafts then was examined weekly with ultrasound. Weekly ultrasound examination of the vein graft wall thickness showed diminished wall thickness at all postoperative time points in vein grafts treated with MMI-0100 (YARAAARQARAKALARQLGVAA; SEQ ID NO: 1), with a ratio of 2.6-fold thicker at 4 weeks, compared to 4.7-fold thicker at 4 weeks in control grafts (FIG. 14, A-B). Histological staining of the grafts confirmed 72% reduced wall thickness with MMI-0100 (YARAAARQARAKALARQLGVAA; SEQ ID NO: 1) treatment compared to control grafts, as seen in vivo with ultrasound (FIGS. 14, C-D). Examination of the grafts for F4/80 immunohistochemical reactivity demonstrated fewer F4/80-positive cells infiltrating into vein grafts treated with MMI-0100 (SEQ ID NO: 1), consistent with fewer infiltrating macrophages in grafts treated with MMI-0100 (YARAAARQARAKALARQLGVAA; SEQ ID NO: 1) (FIGS. 14, E-F). These results in an animal vein graft model suggest that a single ex vivo exposure of the vein graft to MMI-0100 (YARAAARQARAKALARQLGVAA; SEQ ID NO: 1) at the time of surgery inhibits intimal hyperplasia development for several weeks post-surgery.

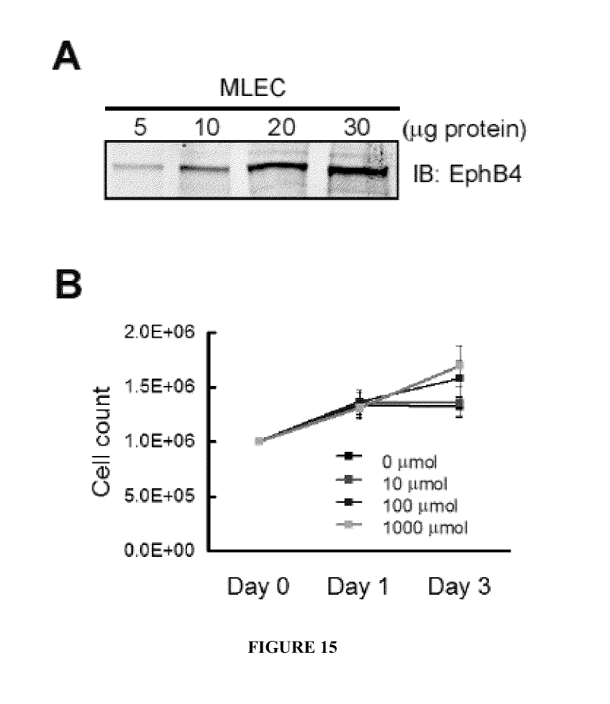

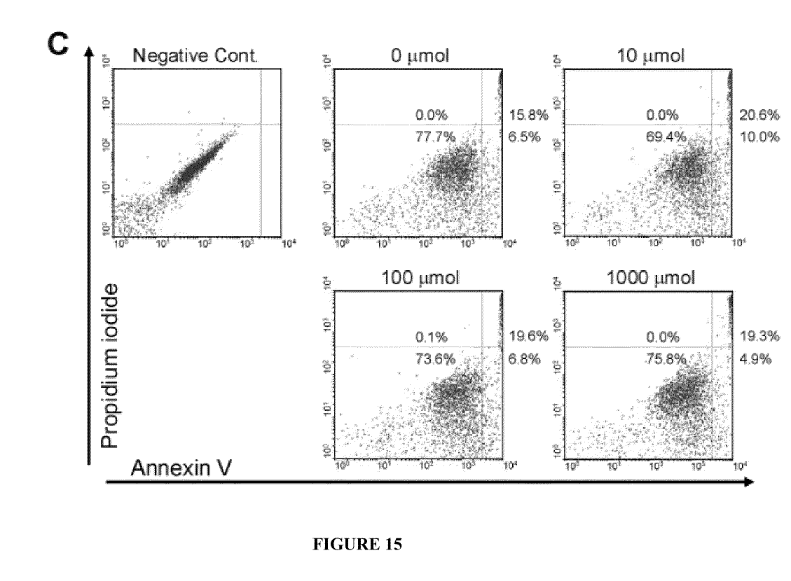

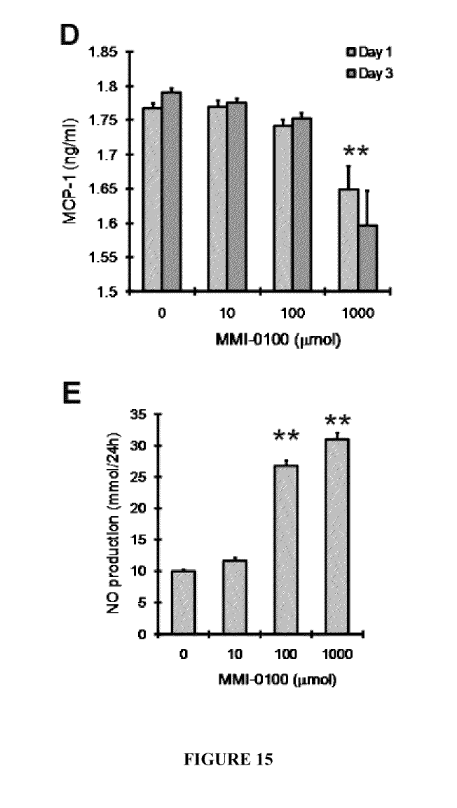

Although MMI-0100 (YARAAARQARAKALARQLGVAA; SEQ ID NO: 1) induces minimal proliferation of human endothelial cells (EC) and smooth muscle cells (SMC) (FIG. 2), the effect was further confirmed by using physiological doses of MMI-0100 (YARAAARQARAKALARQLGVAA; SEQ ID NO: 1) on murine EC. Murine EC were positive for Eph-B4, the marker of venous identity (FIG. 15, A). MMI-0100 (YARAAARQARAKALARQLGVAA; SEQ ID NO: 1) did not induce significant murine EC proliferation at physiological doses (FIG. 15, B). Similarly, MMI-0100 (YARAAARQARAKALARQLGVAA; SEQ ID NO: 1) did not induce EC apoptosis at any dose (FIG. 15, C). MMI-0100 (YARAAARQARAKALARQLGVAA; SEQ ID NO: 1) did not stimulate MCP-1 production, even at high doses (FIG. 15, D), consistent with a reduced number of macrophages in vein grafts treated with MMI-0100 (YARAAARQARAKALARQLGVAA; SEQ ID NO: 1) (FIGS. 14, E-F). Nitric oxide (NO) production was not suppressed, and was even enhanced at physiological doses of MMI-0100 (YARAAARQARAKALARQLGVAA; SEQ ID NO: 1) (FIG. 15, E), suggesting, without being limited by theory, perhaps an additional mechanism of action on endothelial cells.

While the present invention has been described with reference to the specific embodiments thereof it should be understood by those skilled in the art that various changes may be made and equivalents may be substituted without departing form the true sprit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for treating failure of a vascular graft in a subject in need of such treatment, the method comprising administering a therapeutically effective amount of a pharmaceutical composition comprising a polypeptide of amino sequence YARAAARQARAKALARQLGVAA (SEQ ID NO: 1) or a functional equivalent thereof, and a pharmaceutically acceptable carrier.

2. The method according to claim 1, wherein the step of administering is by implanting a biomedical device, wherein the device is a vascular graft, and wherein the composition is disposed on or in the graft.

3. The method according to claim 1, wherein the step of administering occurs parenterally.

4. The method according to claim 1, wherein the step of administering occurs topically.

5. The method according to claim 1, wherein the vascular graft is an autologous graft.

6. The method according to claim 1, wherein the vascular graft is a syngeneic graft.

7. The method according to claim 1, wherein the vascular graft is an allogeneic graft.

8. The method according to claim 1, wherein the vascular graft is a xenograft.

9. The method according to claim 1, wherein the vascular graft is a synthetic graft.

10. The method according to claim 1, wherein the vascular graft is a prosthetic graft.

11. The method according to claim 1, wherein the vascular graft is a tissue engineered graft.

12. The method according to claim 1, wherein the vascular graft is a vascular access graft.

13. The method according to claim 1, wherein the vascular graft is an arteriovenous graft.

14. The method according to claim 1, wherein the vascular graft is a coronary artery bypass graft.

15. The method according to claim 1, wherein the step of administering occurs at one time as a single dose, wherein the one time is during vascular graft surgery.

16. The method according to claim 1, wherein the step of administering is performed as a plurality of doses over a period of time.

17. The method according to claim 16, wherein the period of time is a day, a week, a month, a year, or multiples thereof.

18. The method according to claim 1, wherein the step of administering is performed at least once daily.

19. The method according to claim 1, wherein the step of administering is performed at least once daily for a period of at least one week.

20. The method according to claim 1, wherein the step of administering is performed at least once weekly.

21. The method according to claim 1, wherein the step of administering is performed weekly for a period of at least one month.

22. The method according to claim 1, wherein the step of administering is performed at least once monthly.

23. The method according to claim 1, wherein the method reduces stenosis of the vascular graft.

24. The method according to claim 1, wherein the method reduces vasospasm of at least one blood vessel related to the vascular graft.

25. The method according to claim 1, wherein the method reduces intimal hyperplasia of at least one blood vessel related to the vascular graft.

26. The method according to claim 1, wherein the functional equivalent of the polypeptide YARAAARQARAKALARQLGVAA (SEQ ID NO: 1) has a substantial sequence identity to amino acid sequence YARAAARQARAKALARQLGVAA (SEQ ID NO: 1).

27. The method according to claim 1, wherein the functional equivalent of the polypeptide YARAAARQARAKALARQLGVAA (SEQ ID NO: 1) has at least 70 percent sequence identity to amino acid sequence YARAAARQARAKALARQLGVAA (SEQ ID NO: 1).

28. The method according to claim 1 wherein the functional equivalent of the polypeptide YARAAARQARAKALARQLGVAA (SEQ ID NO: 1) has at least 80 percent sequence identity to amino acid sequence YARAAARQARAKALARQLGVAA (SEQ ID NO: 1).

29. The method according to claim 1, wherein the functional equivalent of the polypeptide YARAAARQARAKALARQLGVAA (SEQ ID NO: 1) has at least 90 percent sequence identity to amino acid sequence YARAAARQARAKALARQLGVAA (SEQ ID NO: 1).

30. The method according to claim 1, wherein the functional equivalent of the polypeptide YARAAARQARAKALARQLGVAA (SEQ ID NO: 1) has at least 95 percent sequence identity to amino acid sequence YARAAARQARAKALARQLGVAA (SEQ ID NO: 1).

31. The method according to claim 1, wherein the functional equivalent of the polypeptide YARAAARQARAKALARQLGVAA (SEQ ID NO: 1) is a polypeptide of amino acid sequence WLRRIKAWLRRIKALNRQLGVAA (SEQ ID NO: 3).

32. The method according to claim 1, wherein the functional equivalent of the polypeptide YARAAARQARAKALARQLGVAA (SEQ ID NO: 1) is a polypeptide of amino acid sequence FAKLAARLYRKALARQLGVAA (SEQ ID NO: 4).

33. The method according to claim 1, the functional equivalent of the polypeptide YARAAARQARAKALARQLGVAA (SEQ ID NO: 1) is a polypeptide of amino acid sequence KAFAKLAARLYRKALARQLGVAA (SEQ ID NO: 5).

34. The method according to claim 1, wherein the functional equivalent of the polypeptide YARAAARQARAKALARQLGVAA (SEQ ID NO: 1) is a polypeptide of amino acid sequence YARAAARQARAKALNRQLGVAA (SEQ ID NO: 6).

35. The method according to claim 1, wherein the functional equivalent of the polypeptide YARAAARQARAKALARQLGVAA (SEQ ID NO: 1) is a polypeptide of amino acid sequence YARAAARQARAKALARQLAVA (SEQ ID NO: 7).

36. The method according to claim 1, wherein the functional equivalent of the polypeptide YARAAARQARAKALARQLGVAA (SEQ ID NO: 1) is a polypeptide of amino acid sequence YARAAARQARAKALARQLGVA (SEQ ID NO: 8).

37. The method according to claim 1, wherein the functional equivalent of the polypeptide YARAAARQARAKALARQLGVAA (SEQ ID NO: 1) is a polypeptide of amino acid sequence YARAAARQARAKALNRQLAVA (SEQ ID NO: 9).

38. The method according to claim 1, wherein the functional equivalent of the polypeptide YARAAARQARAKALARQLGVAA (SEQ ID NO: 1) is a polypeptide of amino acid sequence YARAAARQARAKALNRQLGVA (SEQ ID NO: 10).

39. The method according to claim 1, wherein the functional equivalent of the polypeptide YARAAARQARAKALARQLGVAA (SEQ ID NO: 1) is a polypeptide of amino acid sequence YARAAARQARAKALNRQLGVAA (SEQ ID NO: 11).

40. The method according to claim 1, wherein the functional equivalent of the polypeptide YARAAARQARAKALARQLGVAA (SEQ ID NO: 1) is a polypeptide of amino acid sequence YARAAARQARAKALNRQLAVAA (SEQ ID NO: 12)

41. The method according to claim 1, wherein the functional equivalent of the polypeptide YARAAARQARAKALARQLGVAA (SEQ ID NO: 1) is a fusion protein comprising a first polypeptide operatively linked to a second polypeptide, wherein the first polypeptide is of amino acid sequence YARAAARQARA (SEQ ID NO: 26), and wherein the second polypeptide comprises a therapeutic domain that has a substantial identity to amino acid sequence KALARQLGVAA (SEQ ID NO: 2).

42. The method according to claim 41, wherein the second polypeptide has at least 70 percent sequence identity to amino acid sequence KALARQLGVAA (SEQ ID NO: 2).

43. The method according to claim 41, wherein the second polypeptide has at least 80 percent sequence identity to amino acid sequence KALARQLGVAA (SEQ ID NO: 2).

44. The method according to claim 41, wherein the second polypeptide has at least 90 percent sequence identity to amino acid sequence KALARQLGVAA (SEQ ID NO: 2).

45. The method according to claim 41, wherein the second polypeptide has at least 95 percent sequence identity to amino acid sequence KALARQLGVAA (SEQ ID NO: 2).

46. The method according to claim 41, wherein the second polypeptide is of amino acid sequence KALARQLAVA (SEQ ID NO: 13).

47. The method according to claim 41, wherein the second polypeptide is of amino acid sequence KALARQLGVA (SEQ ID NO: 14).

48. The method according to claim 41, wherein the second polypeptide is of amino acid sequence KALARQLGVAA (SEQ ID NO: 15).

49. The method according to claim 41, wherein the second polypeptide is of amino acid sequence KALNRQLGVAA (SEQ ID NO: 16).

50. The method according to claim 41, wherein the second polypeptide is of the amino acid sequence KAANRQLGVAA (SEQ ID NO: 17).

51. The method according to claim 41, wherein the second polypeptide is of amino acid sequence KALNAQLGVAA (SEQ ID NO: 18).

52. The method according to claim 41, wherein the second polypeptide is of amino acid sequence KALNRALGVAA (SEQ ID NO: 19).

53. The method according to claim 41, wherein the second polypeptide is of amino acid sequence KALNRQAGVAA (SEQ ID NO: 20).

54. The method according to claim 41, wherein the second polypeptide is of amino acid sequence KALNRQLAVA (SEQ ID NO: 21)

55. The method according to claim 41, wherein the second polypeptide is of amino acid sequence KALNRQLAVAA (SEQ ID NO: 22).

56. The method according to claim 41, wherein the second polypeptide is of amino acid sequence KALNRQLGAAA (SEQ ID NO: 23).

57. The method according to claim 41, wherein the second polypeptide is of amino acid sequence KALNRQLGVA (SEQ ID NO: 24).

58. The method according to claim 41, wherein the second polypeptide is of amino acid sequence KKKALNRQLGVAA (SEQ ID NO: 25).

59. The method according to claim 1, wherein the functional equivalent of the polypeptide YARAAARQARAKALARQLGVAA (SEQ ID NO: 1) is a fusion protein comprising a first polypeptide operatively linked to a second polypeptide, wherein the first polypeptide comprises a protein transduction domain functionally equivalent to amino acid sequence YARAAARQARA (SEQ ID NO: 26), and wherein the second polypeptide is of amino acid sequence KALARQLGVAA (SEQ ID NO: 2).

60. The method according to claim 59, wherein the first polypeptide is of amino acid sequence WLRRIKAWLRRIKA (SEQ ID NO: 27).

61. The method according to claim 59, wherein the first polypeptide is of amino acid sequence WLRRIKA (SEQ ID NO: 28).

62. The method according to claim 59, wherein the first polypeptide is of amino acid sequence YGRKKRRQRRR (SEQ ID NO: 29).

63. The method according to claim 59, wherein the first polypeptide is of amino acid sequence WLRRIKAWLRRI (SEQ ID NO: 30).

64. The method according to claim 59, wherein the first polypeptide is of amino acid sequence FAKLAARLYR (SEQ ID NO: 31).

65. The method according to claim 59 wherein the first polypeptide is of amino acid sequence KAFAKLAARLYR (SEQ ID NO: 32).

66. A method for treating a vascular disease comprising intimal hyperplasia in a subject in need of such treatment, the method comprising administering a therapeutically effective amount of a pharmaceutical composition comprising a polypeptide of amino acid sequence YARAAARQARAKALARQLGVAA (SEQ ID NO: 1) or a functional equivalent thereof, and a pharmaceutically acceptable carrier.

67. The method according to claim 66, wherein the vascular disease is a pre-atherosclerotic intimal hyperplasia.

68. The method according to claim 66, wherein the vascular disease is an atherosclerosis.

69. The method according to claim 66, wherein the step of administering is by implanting a biomedical device, wherein the pharmaceutical composition is disposed on or in the device.

70. The method according to claim 66, wherein the step of administering occurs parenterally.

71. The method according to claim 66, wherein the step of administering occurs topically.

72. The method according to claim 66, wherein the step of administering occurs at one time as single dose, wherein the one time is during vascular graft surgery.

73. The method according to claim 66, wherein the step of administering is performed as a plurality of doses over a period of time.

74. The method according to claim 73, wherein the period of time is a day, a week, a month, a year, or multiples thereof.

75. The method according to claim 66, wherein the step of administering is performed at least once daily.

76. The method according to claim 66, wherein the step of administering is performed at least once daily for a period of at least one week.

77. The method according to claim 66, wherein the step of administering is performed at least once weekly.

78. The method according to claim 66, wherein the step of administering is performed weekly for a period of at least one month.

79. The method according to claim 66, wherein the step of administering is performed at least once monthly.

80. The method according to claim 66, wherein the functional equivalent of the polypeptide YARAAARQARAKALARQLGVAA (SEQ ID NO: 1) has a substantial sequence identity to amino acid sequence YARAAARQARAKALARQLGVAA (SEQ ID NO: 1).

81. The method according to claim 66, wherein the functional equivalent of the polypeptide YARAAARQARAKALARQLGVAA (SEQ ID NO: 1) has at least 70 percent sequence identity to amino acid sequence YARAAARQARAKALARQLGVAA (SEQ ID NO: 1).

82. The method according to claim 66, wherein the functional equivalent of the polypeptide YARAAARQARAKALARQLGVAA (SEQ ID NO: 1) has at least 80 percent sequence identity to amino acid sequence YARAAARQARAKALARQLGVAA (SEQ ID NO: 1).

83. The method according to claim 66, wherein the functional equivalent of the polypeptide YARAAARQARAKALARQLGVAA (SEQ ID NO: 1) has at least 90 percent sequence identity to amino acid sequence YARAAARQARAKALARQLGVAA (SEQ ID NO: 1).

84. The method according to claim 66, wherein the functional equivalent of the polypeptide YARAAARQARAKALARQLGVAA (SEQ ID NO: 1) has at least 95 percent sequence identity to amino acid sequence YARAAARQARAKALARQLGVAA (SEQ ID NO: 1).

85. The method according to claim 66, wherein the functional equivalent of the polypeptide YARAAARQARAKALARQLGVAA (SEQ ID NO: 1) is of amino acid sequence WLRRIKAWLRRIKALNRQLGVAA (SEQ ID NO: 3).

86. The method according to claim 66, wherein the functional equivalent of the polypeptide YARAAARQARAKALARQLGVAA (SEQ ID NO: 1) is of amino acid sequence FAKLAARLYRKALARQLGVAA (SEQ ID NO: 4).

87. The method according to claim 66, the functional equivalent of the polypeptide YARAAARQARAKALARQLGVAA (SEQ ID NO: 1) is of amino acid sequence KAFAKLAARLYRKALARQLGVAA (SEQ ID NO: 5).

88. The method according to claim 66, wherein the functional equivalent of the polypeptide YARAAARQARAKALARQLGVAA (SEQ ID NO: 1) is of amino acid sequence YARAAARQARAKALNRQLGVAA (SEQ ID NO: 6).

89. The method according to claim 66, wherein the functional equivalent of the polypeptide YARAAARQARAKALARQLGVAA (SEQ ID NO: 1) is of amino acid sequence YARAAARQARAKALARQLAVA (SEQ ID NO: 7).

90. The method according to claim 66, wherein the functional equivalent of the polypeptide YARAAARQARAKALARQLGVAA (SEQ ID NO: 1) is of amino acid sequence YARAAARQARAKALARQLGVA (SEQ ID NO: 8).

91. The method according to claim 66, wherein the functional equivalent of the polypeptide YARAAARQARAKALARQLGVAA (SEQ ID NO: 1) is of amino acid sequence YARAAARQARAKALNRQLAVA (SEQ ID NO: 9).

92. The method according to claim 66, wherein the functional equivalent of the polypeptide YARAAARQARAKALARQLGVAA (SEQ ID NO: 1) is of amino acid sequence YARAAARQARAKALNRQLGVA (SEQ ID NO: 10).

93. The method according to claim 66, wherein the functional equivalent of the polypeptide YARAAARQARAKALARQLGVAA (SEQ ID NO: 1) is of amino acid sequence YARAAARQARAKALNRQLGVAA (SEQ ID NO: 11).

94. The method according to claim 66, wherein the functional equivalent of the polypeptide YARAAARQARAKALARQLGVAA (SEQ ID NO: 1) is of amino acid sequence YARAAARQARAKALNRQLAVAA (SEQ ID NO: 12)

95. The method according to claim 66, wherein the functional equivalent of the polypeptide YARAAARQARAKALARQLGVAA (SEQ ID NO: 1) is a fusion protein comprising a first polypeptide operatively linked to a second polypeptide, wherein the first polypeptide is of amino acid sequence YARAAARQARA (SEQ ID NO: 26), and wherein the second polypeptide comprises a therapeutic domain that has a substantial identity to amino acid sequence KALARQLGVAA (SEQ ID NO: 2).

96. The method according to claim 95, wherein the second polypeptide has at least 70 percent sequence identity to KALARQLGVAA (SEQ ID NO: 2).

97. The method according to claim 95, wherein the second polypeptide has at least 80 percent sequence identity to KALARQLGVAA (SEQ ID NO: 2).

98. The method according to claim 95, wherein the second polypeptide has at least 90 percent sequence identity to KALARQLGVAA (SEQ ID NO: 2).

99. The method according to claim 95, wherein the second polypeptide has at least 95 percent sequence identity to KALARQLGVAA (SEQ ID NO: 2).

100. The method according to claim 95, wherein the second polypeptide is of amino acid sequence KALARQLAVA (SEQ ID NO: 13).

101. The method according to claim 95, wherein the second polypeptide is of amino acid sequence KALARQLGVA (SEQ ID NO: 14).

102. The method according to claim 95, wherein the second polypeptide is of amino acid sequence KALARQLGVAA (SEQ ID NO: 15).

103. The method according to claim 95, wherein the second polypeptide is of amino acid sequence KALNRQLGVAA (SEQ ID NO: 16).

104. The method according to claim 95, wherein the second polypeptide is of amino acid sequence KAANRQLGVAA (SEQ ID NO: 17).

105. The method according to claim 95, wherein the second polypeptide is of amino acid sequence KALNAQLGVAA (SEQ ID NO: 18).

106. The method according to claim 95, wherein the second polypeptide is of amino acid sequence KALNRALGVAA (SEQ ID NO: 19).

107. The method according to claim 95, wherein the second polypeptide is of amino acid sequence KALNRQAGVAA (SEQ ID NO: 20).

108. The method according to claim 95, wherein the second polypeptide is of amino acid sequence KALNRQLAVA (SEQ ID NO: 21).

109. The method according to claim 95, wherein the second polypeptide is of amino acid sequence KALNRQLAVAA (SEQ ID NO: 22).

110. The method according to claim 95, wherein the second polypeptide is of amino acid sequence KALNRQLGAAA (SEQ ID NO: 23).

111. The method according to claim 95, wherein the second polypeptide is of amino acid sequence KALNRQLGVA (SEQ ID NO: 24).

112. The method according to claim 95, wherein the second polypeptide is of amino acid sequence KKKALNRQLGVAA (SEQ ID NO: 25).

113. The method according to claim 66, wherein the functional equivalent of the polypeptide YARAAARQARAKALARQLGVAA (SEQ ID NO: 1) is a fusion protein comprising a first polypeptide operatively linked to a second polypeptide, wherein the first polypeptide comprises a protein transduction domain functionally equivalent to amino acid sequence YARAAARQARA (SEQ ID NO: 26), and wherein the second polypeptide is of amino acid sequence KALARQLGVAA (SEQ ID NO: 2).

114. The method according to claim 113, wherein the first polypeptide is of amino acid sequence WLRRIKAWLRRIKA (SEQ ID NO: 27).

115. The method according to claim 113, wherein the first polypeptide is of amino acid sequence WLRRIKA (SEQ ID NO: 28).

116. The method according to claim 113, wherein the first polypeptide is of amino acid sequence YGRKKRRQRRR (SEQ ID NO: 29).

117. The method according to claim 113, wherein the first polypeptide is of amino acid sequence WLRRIKAWLRRI (SEQ ID NO: 30).

118. The method according to claim 113, wherein the first polypeptide is of amino acid sequence FAKLAARLYR (SEQ ID NO: 31).

119. The method according to claim 113, wherein the first polypeptide is of amino acid sequence KAFAKLAARLYR (SEQ ID NO: 32).
